(19) United States
(12) Patent Application Publication
(10) Pub. No.: US 2009/0130112 A1
(43) Pub. Date: May 21, 2009

(54) SPATIAL FOR ALTERING CELL PROLIFERATION

(75) Inventors: FRANCIS A. FLOMERFELT, KENSINGTON, MD (US); RONALD E. GRESS, GAITHERSBURG, MD (US)

Correspondence Address: KLARQUIST SPARKMAN, LLP, 121 S.W. SALMON STREET, SUITE #1600, PORTLAND, OR, 97204-2988, US

(73) Assignees: THE GOVERNMENT OF THE UNITED STATES OF AMERICA AS REPRESENTED BY THE; SECRETARY OF THE DEPARTMENT OF HEALTH AND HUMAN SERVICES

(21) Appl. No.: 12/239,466

(22) Filed: Sep. 26, 2008

Related U.S. Application Data
(63) Continuation of application No. 10/579,879, filed on May 17, 2006, now abandoned.

(51) Int. Cl. A61K 39/395; A61K 31/7105; A61K 38/10; A61P 37/06; A61P 37/00

(52) U.S. Cl. 424/139.1; 514/44; 514/14

(57) ABSTRACT

This disclosure provides methods useful for altering cell proliferation by modifying SPATIAL activity in cells. In some methods, thymocyte numbers in subjects with disease-associated immunodeficiencies are increased by administering an agent that inhibits SPATIAL activity. Also provided are methods useful for increasing thymocyte number in a subject by administering an agent that interferes with an interaction between SPATIAL and Uba3. In other methods, cell growth is inhibited by introducing or expressing a SPATIAL or SPATIAL-related polypeptide or nucleic acid in one or more cell(s), such as neoplastic cell(s). Further provided are methods of identifying agents that modify (for example, inhibit) SPATIAL expression or activity, or which interfere with an interaction between SPATIAL and Uba3 polypeptides, and therefore which are useful in influencing thymocyte number.

### REFERENCE TO RELATED APPLICATIONS

This is a continuation of co-pending U.S. application Ser. No. 10/579,879, filed May 17, 2006, which is the §371 U.S. National Stage of International Application No. PCT/US2003/036874, filed Nov. 18, 2003. Each of these applications is incorporated herein in its entirety.

### FIELD

This disclosure relates to methods of altering cell proliferation, for example, altering cell cycle progression and/or stimulating thymocyte number in a subject, by affecting the expression and/or an activity of SPATIAL nucleic acids and/or polypeptides.

### BACKGROUND

The immune system provides primary protection against pathogens in the body. Thus, an immunodeficient subject is vulnerable to a host of infections, some of which may become life threatening or fatal. In particular, T cells are central to adaptive cell-mediated immune responses in which foreign pathogens, virally infected cells, and tumorigenic cells are recognized and destroyed.

The thymus is responsible for the production of T cells in vertebrates (Miller, Lancet, 11:748, 1961). The thymus comprises two broad types of cells. Lymphoid cells, which include thymocytes, are derived from bone marrow stem cells (Mori et al., Blood, 98(3):696-704, 2001) and reside only transiently in the thymus. Cells that permanently populate the thymus are collectively known as stromal cells. Stromal cells interact with each other and form a specialized microenvironment that actively participates in T cell development (Anderson and Jenkinson, Nature Rev. Immunol, 1(1):31-40, 2001; Bleul and Boehm, Eur. J. Immunol., 30(12):3371-3379, 2000).

Progression of thymocyte development is coincident with migration of the cells through histologically distinct compartments of the thymus. As shown schematically in FIG. 1, bone marrow derived stem cells enter the thymus at the cortico-medullary junction and migrate to the subcapsular region (Prockop and Petrie, Semin. Immunol., 12(5):435-444, 2000). In this process, the stem cells differentiate into double negative (DN) cells, which are T cell precursors that do not express CD4 or CD8. In the subcapsule, DN cells sequentially differentiate from the earliest stage, DN1 to the latest stage DN4, and undergo significant proliferation (Penit, J. Histochem. Cytochem., 36(5):473-478, 1988; Penit and Vasseur, J. Immunol., 140(10):3315-3323, 1988; Lind et al., J. Exp. Med., 194(2):127-134, 2001). This expansion of DN cells assures that sufficient numbers of mature T cells are produced (Almeida et al., J. Exp. Med., 194(5):591-599, 2001). Thymocytes undergo further differentiation as they migrate from the subcapsule to the cortex and from the cortex to the medulla (e.g., Scollay et al., Adv. Exp. Med. Biol, 186:229-234, 1985), prior to exiting the thymus as naive T cells (Campbell et al., J. Immunol., 163(5):2353-2357, 1999).

T-cell deficiency may arise as a result of normal physiological processes (such as, aging), or as a result of a pathological condition (such as, HIV infection or severe combined immunodeficiency syndrome (SCID)) or as a result of medical treatment (such as, chemotherapy, radiation therapy, or immunosuppressive drug administration). For example, anti-cancer therapy often involves the use of cytotoxic treatments to kill cancer cells. Bone marrow stem cells and immune system cells (including T cells) in the cancer patient are also killed by these treatments. Reconstitution of the immune system following anti-cancer treatment is crucial for the health and the long-term recovery of patients. Bone marrow transplantation (BMT) in these patients provides stem cells that can enter the thymus and develop into T cells; thus, reconstituting cell-mediated immunity. Unfortunately in adult humans, the production of T cells by the thymus subsequent to BMT is often inefficient due to an age-related decline in thymic function. Even after BMT, the patients experience T cell deficiency lasting 6-12 months. During this critical period, the patients are prone to opportunistic infection and cannot respond effectively to vaccines (Roux et al., Blood, 96(6):2299-2303, 2000).

Neoplasia is another significant medical problem. Neoplasia, which is the pathological process by which tumors develop, involves unregulated, or at best misregulated, cellular growth and division. The molecular pathways that regulate cellular growth must inevitably intersect with those that regulate the cell cycle. The cell cycle consists of a cell division phase and the events that occur during the period between successive cell divisions, known as interphase. Interphase is composed of successive G1, S, and G2 phases, and normally comprises 90% or more of the total cell cycle time. Most cell components are made continuously throughout interphase; it is therefore difficult to define distinct stages in the progression of the growing cell through interphase. One exception is DNA synthesis, since the DNA in the cell nucleus is replicated only during a limited portion of interphase. This period is denoted as the S phase (S=synthesis) of the cell cycle. The other distinct stage of the cell cycle is the cell division phase, which includes both nuclear division (mitosis) and the cytoplasmic division (cytokinesis) that follows. The entire cell division phase is denoted as the M phase (M=mitotic). This leaves the period between the M phase and the start of DNA synthesis, which is called the G1 phase (G=gap), and the period between the completion of DNA synthesis and the next M phase, which is called the G2 phase (Alberts et al., Molecular Biology of the Cell, New York: Garland Publishing, Inc., 1983, pages 611-612).

One pathway that affects cycle control (among other cellular processes) is the neddylation pathway. Nedd8 is an 81 amino acid ubiquitin-like protein that is highly enriched in the nucleus (Yeh et al., Gene, 248:1-14, 2000). A protein complex containing Uba3 conjugates Nedd8 to Cullin-1 (Cul1) (Gong and Yeh, J. Biol. Chem., 274(17):12036-12042, 1999). Cul1 is a part of the Skp\Cul-1F-box (SCF) protein complex that specifically targets phosphorylated proteins for ubiquitination and degradation via the ubiquitin-mediated proteasome pathway (Morimoto et al., Biochem. Biophys. Res. Commun., 270(3):1093-1096, 2000). Importantly, the SCF complex targets proteins substrates required for cell cycle control and signal transduction. For example, the SCF complex is essential for degradation of p27kip1 an important regulator of the mammalian cell cycle (Podust et al., Proc. Natl. Acad. Sci., 97(9):4579-4584, 2000). The Nedd8 pathway provides an additional regulatory step for control of important cellular processes such as cell cycle regulation.

New methods of altering cell proliferation, for example, altering cell cycle progression and/or increasing thymocyte number in a subject are needed.

### SUMMARY OF THE DISCLOSURE

SPATIAL, a gene expressed predominantly in thymus and lymph node, has been recently discovered (Flomerfelt et al., Genes and Immunity, 1:391-401, 2000). SPATIAL activity has now been found to directly or indirectly alter cell proliferation.

In one embodiment, SPATIAL expression has been found to influence and control thymocyte number in disease-associated immunodeficiencies. For example, it has been found that inhibition of SPATIAL expression leads to surprisingly rapid thymocyte accumulation and differentiation in thymii of severely immunodeficient subjects who have received bone marrow transplantation. Thus, this disclosure provides methods for increasing thymocyte number in subjects with disease-associated immunodeficiencies by administering an agent that inhibits SPATIAL activity. In particular examples, an agent inhibits SPATIAL activity to promote proliferation of cells, such as thymic stromal cells, which enhance proliferation and differentiation of thymocytes. It is therefore now possible to more rapidly reconstitute immune function in subjects who have become immunocompromised, for example by a toxin (such a chemotherapeutic agent) or by an infectious disease (such as HIV infection), thereby reducing morbidity and mortality.

Moreover, it has been discovered that SPATIAL is a potent negative cell cycle regulator. It is now possible to use SPATIAL or fragments or variants thereof to affect cell cycle progression, for example, to slow or stop cell cycle progression in cells, such as neoplastic cells.

In some embodiments, SPATIAL regulates the cell cycle via a newly described protein-protein interaction between SPATIAL and Uba3. Though not bound by theory, it is believed that SPATIAL inhibits the formation of an Uba3-containing protein complex that conjugates Nedd8 to Cul1 such that cell cycle inhibitory kinases that block progression of the cell cycle are not degraded leading to cell cycle arrest in the G1 phase. It is now possible to interfere with an interaction between SPATIAL and Uba3 to affect cell cycle progression. In specific examples, an agent that interferes with a SPATIAL/Uba3 interaction promotes proliferation of cells, such as thymic stromal cells, which cells then enhance the production and differentiation of thymocytes. Accordingly, this disclosure provides methods for increasing thymocyte number in a subject by administering an agent that affects an interaction between SPATIAL and Uba3.

Further provided are methods of identifying agents that modify SPATIAL expression or activity (for instance, agents that inhibit or enhance SPATIAL expression or activity), or agents that affect an interaction between SPATIAL and Uba3 polypeptides. Such agents are therefore useful in influencing cellular proliferation (such as, by increasing thymocyte number or influencing the cell cycle).

The foregoing and other features and advantages will become more apparent from the following detailed description of several embodiments, which proceeds with reference to the accompanying figures.

### BRIEF DESCRIPTION OF THE FIGURES

FIG. 1 shows a schematic drawing of the structural organization and progression of thymocyte development in the thymus. DN1-4 represent progressive stages of T cell precursors collectively known as double negative (DN) cells. DN cells do not express either CD4 or CD8 and are found predominantly in the subcapsule. DP represents other T cell precursors called double positive cells, which express both CD4 and CD8 and are found predominantly in the cortex of the thymus. CD8 and CD4 cells, which are localized predominantly in the medulla of the thymus, are also called single positive (or SP) and express the indicated marker.

FIG. 2 shows the absolute number of thymocytes in thymii of aged (10-12 month old) wild type mice, SPATIAL heterozygote mice (i.e., SPATIAL+/−), and SPATIAL null mice (i.e., SPATIAL −/−). This figure demonstrates that thymocyte number is increased over wild type in both SPATIAL heterozygotes and SPATIAL null mice. Thus, thymocyte numbers may be increased with total to less-than-total inhibition of SPATIAL gene expression.

FIG. 3 is a digital autoradiograph showing the results of a GST-pulldown assay demonstrating in vitro binding of a ³⁵S-methionine labeled, Myc-tagged Uba3-clone 346 (Myc-346) protein fragment and GST-SPATIAL long isoform fusion protein (GST-SPATIAL(L)). The lane marked “IVT” shows all radiolabeled proteins present in the T7 in vitro translation reaction employing a Myc-346 fusion construct as a template. The lane marked “IP” shows the outcome of immunoprecipitation of a radiolabeled Myc-346 IVT reaction with anti-Myc antibody (positive control). The lane marked “GST” shows the precipitate from a GST pulidown assay that included GST alone, glutathione beads, and a radiolabeled Myc-346 IVT reaction mixture (negative control). The lane marked “GST-SPATIAL(L)” shows the precipitate from a GST pulldown assay that included GST-SPATIAL(L), glutathione beads, and a radiolabeled Myc-346 IVT reaction mixture.

FIG. 4 is a digital autoradiograph showing the results of a GST pulldown assay demonstrating that SPATIAL blocks the protein-protein interaction between Uba3 and AppBP1. Lane A shows the outcome of an anti-Myc antibody immunoprecipitation of a mixture containing ³⁵S-methionine labeled AppBP1 and a ³⁵S-methionine labeled, Myc-tagged, full-length mouse Uba3. Lane B shows radiolabeled protein in the precipitate of a GST pulldown reaction containing ³⁵S-methionine labeled AppBP1, ³⁵S-methionine labeled, Myc-tagged Uba3, GST-SPATIAL(L) and glutathione beads. Lane C shows the outcome of an anti-Myc antibody immunoprecipitation of the supernatant of the GST pulldown reaction described in Lane B.

FIG. 5 shows schematic representations of full-length Uba3 cDNA (A) and four Uba3 deletion constructs (B-E), and the corresponding digital autoradiographs of GST pulldown assays that included the respective ³⁵S-methionine labeled Uba3 IVT reaction mixture. Construct (B) represents Uba3-clone 346, which was identified in a yeast two hybrid screen using SPATIAL as the prey construct (see, Example 9). The autoradiographs show precipitated protein from GST pulldown assays containing glutathione beads and the respective radiolabeled Uba3 IVT reaction mixture, in each case, and GST alone (in lane “G”), GST-SPATIAL(L) (in lane “L”), or GST-SPATIAL short isoform fusion protein (GST-SPATIAL(S)) (in lane “S”). This figure demonstrates that the interaction between SPATIAL and Uba3 involves at least the amino acids encoded by nucleotides 586-963 of Uba3. The black boxes between nucleotides 165-186 and 642-669 indicate the locations of nucleotides encoding the Uba3 consensus ATP binding site and active site, respectively. Nucleotides numbering is as set forth in SEQ ID NO: 5.

FIG. 6 shows a graph of the number of enhanced green fluorescent protein (EGFP)-positive cells measured by fluorescence activated cell sorting (FACS) 48 through 168 hours after transfection of 293T human kidney epithelial cells (293T cells) with an expression plasmid for EGPF alone, EGFP-SPATIAL long isoform fusion protein (EGFP-SPATIAL(L)), or EGFP-SPATIAL short isoform fusion protein (EGFP-SPATIAL(S)).

FIG. 7 shows graphs of cell number versus intensity of DiI fluorescence. 293T cells were transfected with expression plasmid for EGFP alone (PEGFPN1; Clontech) or EGFP-SPATIAL(L). Twenty four (24) hours later, transfected cells were reacted with 3H-Indolium, 2-[3-(1,3-dihydro-3,3-dimethyl-1-octacdecyl-2H-indol-2-ylidene)-1-propenyl]-3,3-dimethyl-1-octacdecyl-, perchlorate (DiI; Molecular Probes, Eugene, Oreg.) a fluorescent compound that covalently binds to lipids. EGFP-positive and/or EGFP-negative cells were analyzed by FACS on the day after transfection and 1 hour after DiI labeling (Day 0) and four days after transfection (Day 4) as indicated. This figure demonstrates that SPATIAL transfection blocks cell division.

FIG. 8 shows graphs of DNA content in 293T cells transfected with either EGFP-SPATIAL(L) or EGFP expression plasmids. EGFP-positive cells were sorted by FACS at 36 or 48 hours after transfection. Nuclei were prepared from the sorted cells and stained with propodium iodide. DNA content was assessed by FACS analysis, as described by Lacana and D'Adamio (Nal. Med., 5(5):542-547, 1999). This figure demonstrates that SPATIAL expression inhibits cells from entering the G2-S phases of the cell cycle.

FIG. 9 shows a graph of the number of EGFP-positive 293T cells measurable by FACS 24, 72 and 144 hours after transfection of the cells with an expression plasmid for EGPF-SPATIAL(L) alone or after co-transfection of the cells with an EGFP-SPATIAL(L) expression plasmid and increasing amounts of an Uba3 expression plasmid. This figure demonstrates that Uba3 expression overcomes SPATIAL-mediated growth arrest in a dose-dependent manner.

FIG. 10 shows the number of B cells present in the spleens of mice in which both alleles of the recombination activating gene 2 (Rag2) gene have been knocked out (Rag2 null) or mice in which both alleles of both the SPATIAL gene and the Rag2 gene were knocked out (SP/Rag2 DKO) mice three weeks after BMT. This figure illustrates that Rag2 null and SP/Rag2 DKO mice did not differ in their ability to take up donor bone marrow cells.

FIG. 11 shows the total number of donor bone marrow cells present in the thymii of Rag2 null or SP/Rag2 DKO mice three weeks after BMT. This figure illustrates rapid thymic colonization in SP/Rag2 DKO mice after BMT.

FIG. 12 shows the number of DN11, DN2, DN3 and DN4 thymocytes present in thymii three weeks after BMT in Rag2 null and SP/Rag2 DKO mice.

FIG. 13 shows a FACS analysis profile of thymocytes isolated at three week post-BMT from Rag2 null mice and SP/Rag2 DKO mice.

FIG. 14 shows proliferation data from purified T cells exposed to APC and antigen from wild type (closed circle n=3) or SPATIAL null (open circle n=5) mice. This figure demonstrates that T cell response is normal in SPATIAL null mice.

FIG. 15 shows that SPATIAL and Uba3-clone 346 interact in vivo. 293 T human kidney epithelial cells were transfected with an expression vector for Myc-tagged Uba3-clone 346 (Myc-346) alone or with expression vectors for both HA-tagged SPATIAL and Myc-346. FIG. 15A shows a Western blot of proteins that were immunoprecipated by anti-HA antibody from cell lysates of transfected (lanes 1 and 2) and untransfected (lane 3) cells. This first blot was probed with anti-Myc antibody. Myc-346 is not immunoprecipitated by anti-HA antibody in cells transfected with Myc-346 alone (lane 1). In comparison, Myc-346 is immunoprecipitated by anti-HA antibody in cells that were co-transfected with HA-SPATIAL and Myc-346. The lower band observed in each of lanes 1-3 represents the light chain of the anti-HA antibody, which reacts with the labeled secondary antibody.

FIG. 15B shows another Western blot of the sample from FIG. 15A, lane 2, which was probed with anti-SPATIAL antibody. Thus, anti-HA antibody co-immunoprecipates both HA-SPATIAL and Myc-346.

FIG. 16 shows schematic representations of a panel of SPATIAL deletion mutants (A), and a bar graph showing the effects of the respective deletion mutant on cell growth (B). The darkened areas in the schematic representations of panel A indicate the position of the alternatively spliced exon of SPATIAL. Data in panel B is presented relative to the activity of full-length SPATIAL at 72 hours post transfection. This figure illustrates that the carboxy terminus of SPATIAL is involved in its growth suppression activity.

FIG. 17 shows graphs of the number of EGFP-positive cells over time for cells transfected with the indicated constructs. FIG. 17A shows that cells transfected the caspase inhibitor, Z-VAD.FMK, overcame CD8-Flice induced apoptosis (compare filled with open circles). In comparison, Z-VAD.FMK expression did not overcome SPATIAL-mediated growth suppression (compare filled and open triangles). FIG. 17B shows that Bcl-2 expression, which inhibits apoptosis in the subject cells, does not overcome SPATIAL-mediated growth suppression.

FIG. 18 shows a schematic representation of the SPATIAL gene regulatory region (SEQ ID NO: 7) oriented 5′ to 3′ and marked with putative binding sites for selected known DNA-binding proteins or complexes. The 3′ most base in this schematic is −1 with respect to the SPATIAL translation start site; thus, the position numbered 5700 in the schematic represents nucleotide −107 with respect to the SPATIAL translation start site, position 5400 represents nucleotide −407 with respect to the SPATIAL translation start site, etc.

FIG. 19 shows the genomic structure of the SPATIAL gene, the SPATIAL knock out targeting construct, and the genomic structure of the SPATIAL gene following homologous recombination.

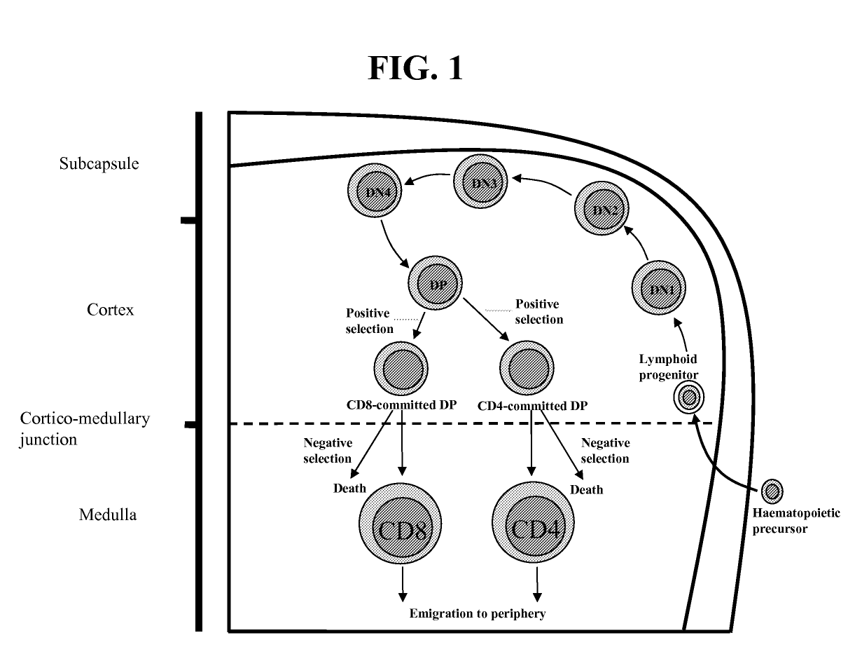

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 shows a nucleic acid sequence of the short isoform of SPATIAL (SPATIAL(S)) (GenBank No. AF257503).

SEQ ID NO: 2 shows the amino acid sequence of the short isoform of SPATIAL.

SEQ ID NO: 3 shows a nucleic acid sequence of the long isoform of SPATIAL (SPATIAL(L)) (GenBank No. AF257502).

SEQ ID NO: 4 shows the amino acid sequence of the long isoform of SPATIAL.

SEQ ID NO: 5 shows a nucleic acid sequence of Uba3.

SEQ ID NO: 6 shows the Uba3 amino acid sequence.

SEQ ID NO: 7 shows the nucleic acid sequence of a SPATIAL gene regulatory region.

### DETAILED DESCRIPTION

### I. Introduction

Disclosed herein are methods of improving immune function in a subject, whose immune function has been compromised by other than age-related immunodeficiency. Immune function is improved by inhibiting a SPATIAL activity in the subject. In some examples, immune function in the subject has been acutely compromised, for instance as a result of administration of a toxin (such as a chemotherapeutic agent) to the subject, infection of the subject with an infectious agent (such as a virus, like HIV), or treatment of the subject with radiation therapy. In other examples, immune function in the subject has been compromised as a result of a disease, such as HIV infection, acquired immunodeficiency syndrome (AIDS), autoimmune disease, thymic hypoplasia, chronic mucocutaneous candidiasis, severe combined immunodeficiency (SCID), cellular immunodeficiency with immunoglobulins (Nezlof syndrome), immunodeficiency with thrombocytopenia and eczema (Wiskott-Aldrich syndrome), ataxia-telangiectasia, immunodeficiency with short-limbed dwarfism, immunodeficiency with thymoma, transcobalamin II deficiency, episodic lymphopenia with lymphotoxin, and idiopathic CD4 lymphocytopenia. Other methods further comprise providing the subject with a bone marrow transplant.

Some methods specifically comprise administering to the subject a therapeutically effective amount of an agent that inhibits a SPATIAL activity to the subject. In certain embodiments, the agent can be a small inhibitory RNA, an anti-sense nucleic acid, a ribozyme, an aptamer, a mirror-image aptamer, an Uba3 peptide, a SPATIAL peptide, an Uba3-specific antibody, or a SPATIAL-specific antibody. In other embodiments, the agent inhibits an interaction between SPATIAL and Uba3 and, in specific examples, may be an Uba3 peptide, a SPATIAL peptide, an Uba3-specific antibody, a SPATIAL-specific antibody, an aptamer or a mirror-image aptamer.

In some methods wherein an interaction between SPATIAL and Uba3 is disrupted, the agent comprises at least 15 consecutive amino acids of SEQ ID NO: 6, such as at least 15 consecutive amino acids between residues 183-308 of SEQ ID NO: 6.

In some methods, inhibiting SPATIAL activity results in increasing thymocyte number in the subject, for example, increasing DN thymocyte number. In other methods, SPATIAL activity is inhibited by inhibiting SPATIAL gene expression, for example, by substantially eliminating SPATIAL gene expression. In still other methods, SPATIAL activity is inhibited by inhibiting a SPATIAL polypeptide activity.

This specification also discloses methods of increasing thymocyte number in subjects having disease-associated T cell deficiency. Such methods include administering to the subject a therapeutically effective amount of an agent that inhibits SPATIAL activity.

In some methods, inhibition of SPATIAL activity includes inhibiting SPATIAL gene expression. In more particular embodiments, SPATIAL gene expression is substantially eliminated. In other embodiments, the agent that inhibits SPATIAL gene expression includes a small inhibitory RNA (siRNA), an anti-sense nucleic acid, or a ribozyme.

In some embodiments, increasing thymocyte number includes increasing DN thymocyte number in the subject's thymus.

In some methods, the T cell deficiency comprises cellular immunodeficiency or combined immunodeficiency. In particular examples, the disease-associated T cell deficiency is HIV infection, acquired immunodeficiency syndrome (AIDS), thymic hypoplasia, chronic mucocutaneous candidiasis, severe combined immunodeficiency (SCID), cellular immunodeficiency with immunoglobulins (Nezlof syndrome), immunodeficiency with thrombocytopenia and eczema (Wiskott-Aldrich syndrome), ataxia-telangiectasia, immunodeficiency with short-limbed dwarfism, immunodeficiency with thymoma, transcobalamin II deficiency, or episodic lymphopenia with lymphotoxin. In more particular examples, the subject has HIV infection or acquired immunodeficiency syndrome (AIDS).

In some examples of the disclosed methods, the subject has received a bone marrow transplant, chemotherapy or radiation therapy. In particular embodiments wherein the subject has received a bone marrow transplant, mature donor T cells are measurable in the blood of the subject prior to the time mature donor T cells are measurable in the blood of a second bone marrow transplant subject who did not receive an agent that inhibits SPATIAL activity. In other embodiments, the agent is administered prior to the bone marrow transplant, concurrent with the bone marrow transplant, or after the bone marrow transplant. In still other embodiments, the agent is administered a sufficient period of time prior to bone marrow transplant to condition the thymus, such as up to about 30 days before the bone marrow transplant.

In some of the disclosed methods, the agent that inhibits SPATIAL activity and results in increased thymocyte number includes a small inhibitory RNA, an anti-sense nucleic acid, a ribozyme, an aptamer, a mirror-image aptamer, an Uba3 peptide, a SPATIAL peptide, an Uba3-specific antibody, or a SPATIAL-specific antibody. In specific methods, inhibiting SPATIAL activity includes inhibiting SPATIAL polypeptide activity, for example with an agent that inhibits an interaction between SPATIAL and Uba3.

Also disclosed herein are methods of increasing thymocyte number in a subject, which include administering to the subject a therapeutically effective amount of an agent that interferes with the interaction between SPATIAL and Uba3. In particular embodiments, thymocyte numbers are increased, and in specific examples, DN thymocyte numbers in the subject's thymus are increased.

In some methods, the agent inhibits or enhances the interaction between SPATIAL and Uba3. In particular examples, the agent inhibits the interaction between SPATIAL and Uba3.

Examples of suitable agents that affect the interaction between SPATIAL and Uba3 include an Uba3 peptide, a SPATIAL peptide, an Uba3-specific antibody, a SPATIAL-specific antibody, an aptamer or a mirror-image aptamer. In some examples, the agent includes at least 15 consecutive amino acids of SEQ ID NO: 6, such as at least 15 consecutive amino acids between residues 183-308 of SEQ ID NO: 6.

In some methods of increasing thymocyte number by interfering with an interaction between SPATIAL and Uba3, the subject is immunodeficient. In particular methods, the immunodeficiency is cellular immunodeficiency or combined immunodeficiency. In more particular methods, the immunodeficiency is an age-related immunodeficiency. In other embodiments, the subject has received a bone marrow transplant, chemotherapy or radiation therapy. In still other embodiments, the subject has a disorder selected from autoimmune disease, HIV infection, acquired immunodeficiency syndrome (AIDS), thymic hypoplasia, chronic mucocutaneous candidiasis, severe combined immunodeficiency (SCID), cellular immunodeficiency with immunoglobulins (Nezlof syndrome), immunodeficiency with thrombocytopenia and eczema (Wiskott-Aldrich syndrome), ataxia-telangiectasia, immunodeficiency with short-limbed dwarfism, immunodeficiency with thymoma, transcobalamin II deficiency, episodic lymphopenia with lymphotoxin, or idiopathic CD4 lymphocytopenia.

This specification further discloses methods for identifying an agent with potential for increasing thymocyte numbers by determining SPATIAL inhibitory activity of the agent. Some methods further include determining whether administration of the agent to a Rag2 null mouse results in the presence of naive T cells in the blood of the mouse after the mouse receives a bone marrow transplant.

Also disclosed herein are further methods of identifying an agent with potential for increasing thymocyte numbers by providing an Uba3 polypeptide, a fragment thereof, or a functional variant thereof as a first component, and providing a SPATIAL polypeptide, a fragment thereof, or a functional variant thereof as a second component, then contacting the first component and the second component with an agent under conditions that would permit the first and second components to interact in the absence of the agent; and determining whether the agent interferes with the interaction between the first and second components, wherein interfering with the interaction between the first and second components identifies the agent as one that has potential for increasing thymocyte numbers.

In particular methods, the first component contains at least 15 consecutive amino acids of SEQ ID NO: 6 or at least 15 consecutive amino acids of a polypeptide having 80% sequence identity with SEQ ID NO: 6. In more particular methods, the first component contains at least 15 consecutive amino acids between residues 183-308 of SEQ ID NO: 6.

In other embodiments, the second component contains at least 15 consecutive amino acids of SEQ ID NOs: 2 or 4 or at least 15 consecutive amino acids of a polypeptide having 80% sequence identity with SEQ ID NOs: 1 or 3.

Also disclosed herein are methods of influencing cell growth by modifying a SPATIAL activity in at least one cell. In some examples of these methods, SPATIAL activity is increased, and, in particular examples, cell growth (such as neoplastic cell growth) is inhibited. In other methods of influencing cell growth, SPATIAL activity is inhibited, and, in particular, embodiments cell growth is enhanced. In some methods, enhanced cell growth in the cell(s) results in increased thymocyte numbers.

This disclosure further describes methods of inhibiting cell growth by introducing into at least one cell (1) an amino acid sequence which is at least 80% sequence identity with SEQ ID NOs: 2 or 4 and has an activity of SPATIAL; (2) a conservative variant of SEQ ID NOs: 2 or 4 that has an activity of SPATIAL; (3) a fragment of at least fifteen consecutive amino acid residues of SEQ ID NOs: 2 or 4 that has an activity of SPATIAL; (4) at least residues 21-197, at least residues 91-197 or at least residues 145-197 of SEQ ID NO: 2; (5) at least residues 21-231, at least residues 91-176, or at least residues 91-231 of SEQ ID NO: 4; or (6) SEQ ID NOs: 2 or 4. Particular methods are directed to neoplastic cells.

Also disclosed herein are methods of inhibiting cell growth by expressing in at least one cell (1) a nucleic acid sequence having at least 80% sequence identity with SEQ ID NOs: 1 or 3, which encodes a polypeptide having an activity of SPATIAL; (2) a nucleic acid sequence comprising at least fifteen consecutive residues of SEQ ID NOs: 1 or 3, which encodes a polypeptide having an activity of SPATIAL; (3) a nucleic acid sequence comprising at least residues 144-674, at least residues 354-674, or at least residues 516-674 of SEQ ID NO: 1; (4) a nucleic acid sequence comprising at least residues 144-776, at least residues 354-611, or at least residues 354-776 of SEQ ID NO: 3; or (5) a nucleic acid sequence comprising SEQ ID NOs: 1 or 3. Particular methods are directed to neoplastic cells.

Further described herein are methods of treating neoplasia in a subject by administering to a subject a therapeutically effective amount of a cell cycle inhibitory agent which includes: (1) an amino acid sequence which is at least 80% homologous to SEQ ID NOs: 2 or 4 and has an activity of SPATIAL; (2) a conservative variant of SEQ ID NOs: 2 or 4 that has an activity of SPATIAL; (3) a fragment of at least fifteen consecutive amino acid residues of SEQ ID NOs: 2 or 4 that has an activity of SPATIAL; (4) at least residues 21-197, at least residues 91-197 or at least residues 145-197 of SEQ ID NO: 2; (5) at least residues 21-231, at least residues 91-176, or at least residues 91-231 of SEQ ID NO: 4; or (6) SEQ ID NOs: 2 or 4.

### II. Abbreviations and Terms

APC antigen presenting cell

BMT bone marrow transplant

CDR complementarity determining region

DKO double knock out

DN a thymocyte double negative for markers CD4 and CD8

EGFP enhanced green fluorescent protein

FACS fluorescence activated cell sorting

FTOC fetal thymic organ culture

GST glutathione-S-transferase

GST-SPATIAL(L) GST-SPATIAL long isoform fusion protein

GST-SPATIAL(S) GST-SPATIAL short isoform fusion protein

IVT in vitro translation

Myc-346 Myc-tagged Uba3-clone 346

PAGE polyacrylamide gel electrophoresis

PSC-oligo(s) phosphorothioate chimeric oligonucleotide(s)

RACE rapid amplification of cDNA ends

RTOC reaggregate thymic organ culture

RT-PCR reverse transcriptase and polymerase chain reaction

SCID severe combined immunodeficiency syndrome

SPATIAL(L) long isoform of SPATIAL

SPATIAL(S) short isoform of SPATIAL

Z-VAD.FMK benzyloxycarbonyl-valinyl-alaninyl-aspartyl fluoromethylketone

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments disclosed herein, the following explanations of specific terms are provided:

Acute: Having a rapid onset as contrasted to long-term or chronic conditions (such as age-related decrease in immune function). Examples of acute conditions include, without limitation, acute toxic insults, such as chemotherapeutic or radiologic ablation of immune system cells, as may occur, for example, as a treatment prior to bone marrow transplantation.

Agent: Any substance (such as, an atom, molecule, molecular complex, chemical, peptide, protein, protein complex, nucleic acid, or drug) or any combination of substances that is useful for achieving an end or result; for example, a substance or combination of substances useful for inhibiting gene expression or inhibiting protein activity, or useful for modifying or interfering with protein-protein interactions. Similarly, a “component” is any substance (such as, an atom, molecule, molecular complex, chemical, peptide, protein, protein complex, nucleic acid, or drug) that is useful for achieving an end or result.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Antibody: An intact immunoglobulin or an antigen-binding portion thereof. Antigen-binding portions may be produced by recombinant DNA techniques or by enzymatic or chemical cleavage of intact immunoglobulins. Antigen-binding portions include, inter alia, Fab, Fab′, F(ab′)₂, Fv, dAb (Fd), and complementarity determining region (CDR) fragments, single-chain antibodies (scFv), chimeric antibodies, diabodies and polypeptides (including fusion proteins) that contain at least a portion of an immunoglobulin that is sufficient to confer specific antigen binding to the polypeptide. A Fab fragment is a monovalent fragment consisting of the VL, VH, CL and CH1 domains; an F(ab′)₂ fragment is a bivalent fragment comprising two Fab fragments linked by a disulfide bridge at the hinge region; an Fd fragment consists of the VH and CH1 domains; an Fv fragment consists of the VL and VH domains of a single arm of an antibody; and a dAb fragment consists of a VH domain (see, e.g., Ward et al., Nature, 341:544-546, 1989).

The terms “bind specifically” and “specific binding” refer to the ability of a specific binding agent (such as, an antibody) to bind to a target molecular species in preference to binding to other molecular species with which the specific binding agent and target molecular species are admixed. A specific binding agent is said specifically to “recognize” a target molecular species when it can bind specifically to that target.

A “single-chain antibody” (scFv) is a genetically engineered molecule containing the VH and VL domains of one or more antibody(ies) linked by a suitable polypeptide linker as a genetically fused single chain molecule (see, e.g., Bird et al., Science, 242:423-426, 1988; Huston et al., Proc. Natl. Acad. Sci., 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., Proc. Natl. Acad. Sci., 90:6444-6448, 1993; Poljak et al., Structure, 2:1121-1123, 1994). One or more CDRs may be incorporated into a molecule either covalently or noncovalently to make the resultant molecule an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

A “neutralizing antibody” or “an inhibitory antibody” is an antibody that inhibits at least one activity of a polypeptide, such as by blocking the binding of the polypeptide to a ligand to which it normally binds, or by disrupting or otherwise interfering with a protein-protein interaction of the polypeptide with a second polypeptide. An “activating antibody” is an antibody that increases an activity of a polypeptide.

Aptamer: A single-stranded nucleic acid molecule (such as, DNA or RNA) that assumes a specific, sequence-dependent shape and binds to a target protein with high affinity and specificity. Aptamers generally comprise fewer than 100 nucleotides, fewer than 75 nucleotides, or fewer than 50 nucleotides. “Mirror-image aptamer(s)” (also called Spiegelmers™) are high-affinity L-enantiomeric nucleic acids (for example, L-ribose or L-2′-deoxyribose units) that display high resistance to enzymatic degradation compared with D-oligonucleotides (such as, aptamers). The target binding properties of mirror-image aptamers are designed by an in vitro-selection process starting from a random pool of oligonucleotides, as described for example, in Wlotzka et al., Proc. Natl. Acad. Sci. 99(13):8898-8902, 2002. Applying this method, high affinity mirror-image aptamers specific for a polypeptide (such as, SPATIAL) can be generated.

Bone marrow transplant (or hematopoietic stem cell transplant): A procedure in which hematopoietic stem cells found in the bone marrow and/or circulating blood from a donor are transplanted into a recipient. Hematopoietic stem cells give rise to blood cells, including red blood cells, myeloid cells, white blood cells (for example, lymphocytes, such as T cells and thymocytes), and platelets. Bone marrow transplantation is used as a treatment option in many circumstances, including for example, treatments for cancer, blood disorders, and some genetic or inherited illnesses. For instance, many cancer therapies involve high doses of chemotherapy (with or without radiation) to destroy cancer cells; however, this therapy also destroys the chemotherapy recipient's bone marrow and existing immune system. As one consequence, the chemotherapy recipient's hematopoietic stem cells and mature immune cells are intentionally and acutely ablated and the immune system is compromised.

In a bone marrow transplant, healthy bone marrow and/or blood cells and/or mobilized hematopoietic stem cells from a donor are infused into a recipient as a source of hematopoietic stem cells to recover or facilitate bone marrow function, including formation of immune system cells, such as T cells. When a recipient's own hematopoietic stem cells are collected for transplant it is called an “autologous” transplant. If the hematopoietic stem cells are collected from a donor it is called an “allogeneic” transplant.

Cell cycle: The physiological and morphological progression of changes that cells undergo when dividing. The cell cycle consists of a cell division phase and the events that occur during the period between successive cell divisions, known as interphase. Interphase is composed of successive G1, S, and G2 phases, and normally comprises 90% or more of the total cell cycle time. Most cell components are made continuously throughout interphase; it is therefore difficult to define distinct stages in the progression of the growing cell through interphase. One exception is DNA synthesis, since the DNA in the cell nucleus is replicated only during a limited portion of interphase. This period is denoted as the S phase (S=synthesis) of the cell cycle. The other distinct stage of the cell cycle is the cell division phase, which includes both nuclear division (mitosis) and the cytoplasmic division (cytokinesis) that follows. The entire cell division phase is denoted as the M phase (M=mitotic). This leaves the period between the M phase and the start of DNA synthesis, which is called the G1 phase (G=gap), and the period between the completion of DNA synthesis and the next M phase, which is called the G2 phase (Alberts et al., Molecular Biology of the Cell, New York: Garland Publishing, Inc., 1983, pages 611-612).

Condition the thymus: To affect the thymus in such a way that thymocyte development is enhanced in a conditioned thymus as compared to that thymus prior to conditioning. A thymus may be conditioned in any circumstances where it is desirable to enhance thymocyte development, such as before, during or after a bone marrow transplant. For example, the thymus may be conditioned prior to a bone marrow transplant so that donor stem cells that enter the conditioned thymus divide, differentiate, develop and/or accumulate at a faster rate and/or in greater numbers than would occur in a non-conditioned thymus. Effectors that enhance thymocyte development in the thymus include, for example, agents that inhibit SPATIAL activity, or agents that interfere with an interaction between SPATIAL and Uba3.

Gene expression: The process by which the coded information of a nucleic acid transcriptional unit (including, for example, genomic DNA or cDNA) is converted into an operational, non-operational, or structural part of a cell, often including the synthesis of a protein. Gene expression can be influenced by external signals; for instance, exposure of a subject to an agent that inhibits gene expression, such as inhibition of SPATIAL gene expression. Expression of a gene also may be regulated anywhere in the pathway from DNA to RNA to protein. Regulation of gene expression occurs, for instance, through controls acting on transcription, translation, RNA transport and processing, degradation of intermediary molecules such as mRNA, or through activation, inactivation, compartmentalization or degradation of specific protein molecules after they have been made, or by combinations thereof. Gene expression may be measured at the RNA level or the protein level and by any method known in the art, including Northern blot, RT-PCR, Western blot, or in vitro, in situ, or in vivo protein activity assay(s).

The expression of a nucleic acid may be modulated compared to a control state, such as at a control time (for example, prior to administration of a substance or agent that affects regulation of the nucleic acid under observation) or in a control cell or subject, or as compared to another nucleic acid. Such modulation includes but is not necessarily limited to overexpression, underexpression, or suppression of expression. In addition, it is understood that modulation of nucleic acid expression may be associated with, and in fact may result in, a modulation in the expression of an encoded protein or even a protein that is not encoded by that nucleic acid.

“Interfering with or inhibiting gene expression” refers to the ability of an agent to measurably reduce the expression of a target gene. Expression of a target gene may be measured by any method known to those of skill in the art, including for example measuring mRNA or protein levels. It is understood that interfering with or inhibiting gene expression is relative, and does not require absolute suppression of the gene. Thus, in certain embodiments, interfering with or inhibiting gene expression of a target gene requires that, following application of an agent, the gene is expressed at least 5% less than prior to application, at least 10% less, at least 15% less, at least 20% less, at least 25% less, or even more reduced. Thus, in some particular embodiments, application of an agent reduces expression of the target gene by about 30%, about 40%, about 50%, about 60%, or more. In specific examples, where the agent is particularly effective, expression is reduced by 70%, 80%, 85%, 90%, 95%, or even more. Gene expression is “substantially eliminated” when expression of the gene is reduced by 90%, 95%, 98%, 99% or even 100%.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acid sequences or two distinct regions of the same nucleic acid sequence. For example, an oligonucleotide can be complementary to a SPATIAL-encoding mRNA, or an SPATIAL-encoding dsDNA.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6×SSC, 5×Denhardt's solution, 0.5% SDS and 100 μg sheared salmon testes DNA, followed by 15-30 minute sequential washes at 65° C. in 2×SSC, 0.5% SDS, followed by 1×SSC, 0.5% SDS and finally 0.2×SSC, 0.5% SDS.

Improving immune function: Increasing or enhancing the quality or condition of the immune system; for example, by increasing the number of thymocytes. Improvement in immune function is a characteristic that is recognized by those of skill in the art. Such improvement may be detected by measuring known markers of immune system function, such as T cell number, or by observing a subject's resistance (or increased resistance) to diseases that are known to afflict persons with immune deficiency (such as opportunistic infection).

Immunodeficiency (or immunodeficient): A lack of adequate defense against infection caused by a defective (for example, compromised, damaged or ineffective) immune system. The immune system can become defective, for example, as a result of infection by certain viruses such as HIV (believed to be the causative agent of AIDS) or following irradiation or chemotherapy or other drug treatments, or the immune system can be ineffective in severely premature babies or can become ineffective during aging and particularly in advanced old age. “Cellular immunodeficiency” is a deficiency in cell-mediated immunity as a result of T cell deficiency. “Combined immunodeficiency” is a deficiency of lymphoid cells that mediate both humoral (B cell) and cell-mediated (T cell) immunity. Examples of cellular immunodeficiency disorders include, without limitation, HIV infection, AIDS, thymic hypoplasia (DiGeorge syndrome), chronic mucocutaneous candidiasis, and idiopathic CD4 lymphocytopenia. Examples of combined immunodeficiency disorders include, without limitation, severe combined immunodeficiency (SCID), cellular immunodeficiency with immunoglobulins (Nezlof syndrome), immunodeficiency with thrombocytopenia and eczema (Wiskott-Aldrich syndrome), ataxia-telangiectasia, immunodeficiency with short-limbed dwarfism, immunodeficiency with thymoma, transcobalamin II deficiency, and episodic lymphopenia with lymphotoxin. “Age-related immunodeficiency” refers to a gradual, progressive, naturally occurring, non-pathological decline in thymocyte production that begins in the adulthood of a subject and slowly continues as a subject ages.

Influencing the cell cycle: To alter or modify the progression of the cell cycle in a particular cell or population of cells. For example, in quiescent cells, the cell cycle may be influenced by prompting entry of the cells into the cell cycle. Agents, such as SPATIAL inhibitory agents, that result in de-inhibition of cell cycle progression may prompt cells to divide. In dividing cells, such as hyperproliferative cells, the cell cycle may be influenced by inhibiting progression of the cell cycle. “Cell cycle inhibition” (also, “inhibition of cell growth”) means to slow or stop cell cycle progression in a cell or population of cells. The phrase “cell cycle inhibition” is not intended to be an absolute term. Instead, the phrase is intended to convey a wide-range of inhibitory effects that various agents may have on the normal (for example, uninhibited or control) cell cycle. For instance, the cell cycle in a cell population treated with an agent is inhibited when the rate of cell division in the cell population is decreased by at least 10%, at least 20%, at least 30%, at least 50%, at least 80%, or at least 90% as compared to the rate of cell division in the population prior to addition of the agent. In specific examples, the cell cycle is inhibited by arresting a cell (or a portion of a population of cells) in the G1 phase of the cell cycle.

Inhibiting protein activity: To decrease, limit, or block an action, function or expression of a protein. The phrase “inhibiting protein activity” is not intended to be an absolute term. Instead, the phrase is intended to convey a wide-range of inhibitory effects that various agents may have on the normal (for example, uninhibited or control) protein activity. Thus, protein activity may be inhibited when the level or activity of any direct or indirect indicator of the protein's activity is changed (for example, increased or decreased) by at least 10%, at least 20%, at least 30%, at least 50%, at least 80%, at least 100% or at least 250% as compared to control measurements of the same indicator.

Inhibition of protein activity may, but need not, result in an increase in the level or activity of an indicator of the protein's activity. By way of example, this can happen when the protein of interest is acting as an inhibitor or suppressor of a downstream indicator.

Inhibition of protein activity may also be effected, for example, by inhibiting expression of the gene encoding the protein or by decreasing the half-life of the mRNA encoding the protein.

Interaction between SPATIAL and Uba3: A protein-protein interaction between SPATIAL and Uba3. Protein-protein interaction is characterized by physical contact between at least two proteins that is of sufficient affinity and specificity that, for example, immunoprecipitation of one of the proteins will also specifically precipitate the other protein(s); provided that the immunoprecipitating antibody does not also affect the site(s) involved in the protein-protein interaction. Other methods of identifying protein-protein interactions include the yeast two-hybrid system (e.g., Fields and Song, Nature, 340:245-246, 1989; Fields and Sternglanz, Trends Genet., 10(8):286-292, 1994) and the GST pulldown assay (e.g., Ausubel et al., Current Protocols in Molecular Biology, Greene Publishing Associates, updated November 2003, Chapter 20, Analysis of Protein Interactions, Unit 20.2, Affinity Purification of Proteins Binding to GST Fusion Proteins).

Interfere with [an interaction between SPATIAL and Uba3]: To alter or change from one state or condition to another; for example, to weaken, disrupt, or inhibit an interaction between SPATIAL and Uba3. In some examples, an interaction may be modified so as to completely disrupt the interaction, in which event the proteins involved in the interaction would not substantially interact under conditions that would normally permit the interaction. In other examples, an interaction may be weakened so that the proteins involved in the interaction do not interact as strongly as compared to an interaction between the proteins under control conditions.

Isolated: An “isolated” biological component (such as a nucleic acid molecule, protein, antibody or organelle) has been separated or purified away from other biological components in the cell of the organism in which the component naturally occurs, for instance, other chromosomal and extra-chromosomal DNA and RNA, proteins, antibodies and organelles. Nucleic acids and proteins that have been “isolated” include nucleic acids and proteins purified by standard purification methods. The term also embraces nucleic acids and proteins prepared by recombinant expression in a host cell, as well as chemically synthesized biopolymers. The term “isolated” does not require absolute isolation. Similarly, the term “substantially separated” does not require absolute separation.

Lymphocyte: Any of the mononuclear nonphagocytic leukocytes, found in the blood, lymph, and lymphoid tissues (such as the thymus), that are the body's immunologically competent cells and their precursors. Lymphocytes are divided on the basis of ontogeny and function into at least two classes, B and T lymphocytes (a.k.a., B and T cells), which are responsible for humoral and cellular immunity, respectively.

Modulating thymocyte number: To change the number of thymocytes present in the thymus of a subject as compared to a control time point in the same subject or as compared to a second subject that serves as a control. Thymocyte number in either control circumstance being referred to as “the control number of thymocytes.” Modulating thymocyte number encompasses increasing or decreasing thymocyte numbers from the control number of thymocytes. Where expressly indicated, modulating thymocyte number may refer to changing the number of a particular subset of thymocytes, for example, as in “modulating the number of DN thymocytes.”

As used herein, the phrase “increasing thymocyte number” means resulting in more thymocytes as compared to the control number of thymocytes; for example, thymocyte numbers may be at least 10%, at least 25%, at least 50%, at least 100% or at least 250% higher than control, or in some examples even at least 10% higher than control. “Decreasing thymocyte number” means resulting in fewer thymocytes as compared to the control number of thymocytes; for example, thymocyte numbers may be at least 10%, at least 25%, at least 50%, at least 75% or at least 90% fewer than compared to control.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term “nucleic acid molecule” also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, the left hand end of a polynucleotide sequence written in the sense orientation is the 5′ end and the right hand end of the sequence is the 3′ end. In addition, the left hand direction of a polynucleotide sequence written in the sense orientation is referred to as the 5′ direction, while the right hand direction of the polynucleotide sequence is referred to as the 3′ direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

An “anti-sense nucleic acid” is a nucleic acid (such as, an RNA or DNA oligonucleotide) that has a sequence complementary to a second nucleic acid molecule (for example, an mRNA molecule). An anti-sense nucleic acid will specifically bind with high affinity to the second nucleic acid sequence. If the second nucleic acid sequence is an mRNA molecule, for example, the specific binding of an anti-sense nucleic acid to the mRNA molecule can prevent or reduce translation of the mRNA into the encoded protein or decrease the half life of the mRNA, and thereby inhibit the expression of the encoded protein.

Oligonucleotide: A nucleic acid molecule generally comprising a length of 200 bases or fewer. The term often refers to single-stranded deoxyribonucleotides, but it can refer as well to single- or double-stranded ribonucleotides, RNA:DNA hybrids and double-stranded DNAs, among others. In some examples, oligonucleotides are about 10 to about 90 bases in length, for example, 12, 13, 14, 15, 16, 17, 18, 19 or 20 bases in length. Other oligonucleotides are about 25, about 30, about 35, about 40, about 45, about 50, about 55, about 60 bases, about 65 bases, about 70 bases, about 75 bases or about 80 bases in length. Oligonucleotides may be single-stranded, for example, for use as probes or primers, or may be double-stranded, for example, for use in the construction of a mutant gene. Oligonucleotides can be either sense or anti-sense oligonucleotides. An oligonucleotide can be derivatized or modified as discussed above in reference to nucleic acid molecules.

Ribozyme: RNA molecules with enzyme-like properties, which can be designed to cleave specific RNA sequences. Ribozymes are also known as RNA enzymes or catalytic RNAs.

RNA interference (or, RNA silencing or RNAi): A highly conserved gene-silencing mechanism whereby specific double-stranded RNA (dsRNA) trigger the degradation of homologous mRNA (also called, target RNA). Double-stranded RNA is processed into small interfering RNAs (siRNA), which serve as a guide for cleavage of the homologous mRNA in the RNA-induced silencing complex (RISC). The remnants of the target RNA may then also act as siRNA; thus resulting in a cascade effect.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl Math., 2:482, 1981); Needleman and Wunsch (J. Mol. Biol, 48:443, 1970); Pearson and Lipman (Proc. Natl. Acad. Sci., 85:2444, 1988); Higgins and Sharp (Gene, 73:237-244, 1988); Higgins and Sharp (CABIOS, 5:151-153, 1989); Corpet et al (Nuc. Acids Res., 16:10881-10890, 1988); Huang et al (Comp. Appls Biosci., 8:155-165, 1992); and Pearson et al (Meth. Mol. Biol, 24:307-331, 1994). Altschul et al (Nature Genet., 6:119-129, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program© 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the NCSA website. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). When aligning short peptides (fewer than around 30 amino acids), the alignment should be performed using the “Blast 2 sequences” function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. and States, Nature Genet., 3:266-272, 1993; Madden et al., Meth. Enzymol., 266:131-141, 1996; Altschul et al., Nucleic Acids Res., 25:3389-3402, 1997; and Zhang and Madden, Genome Res., 7:649-656, 1997.

Orthologs (equivalent to proteins of other species) of proteins are in some instances characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of one or both binding domains of the disclosed fusion proteins.

When significantly less than the entire sequence is being compared for sequence identity, homologous sequences will typically possess at least 80% sequence identity over short windows of 10-20, and may possess sequence identities of at least 85%, at least 90%, at least 95%, or at least 99% depending on their similarity to the reference sequence. Sequence identity over such short windows can be determined using LFASTA; methods are described at the NCSA website. One of skill in the art will appreciate that these sequence identity ranges are provided for guidance only; it is entirely possible that strongly significant homologs could be obtained that fall outside of the ranges provided. Similar homology concepts apply for nucleic acids as are described for protein.

An alternative indication that two nucleic acid molecules are closely related is that the two molecules hybridize to each other under stringent conditions. Hybridization conditions have been discussed previously.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Small inhibitory RNA (siRNA): A double-stranded RNA molecule, usually less than about 40 nucleotides long, which is an intermediate in RNA interference.

Specific binding agent: An agent that binds substantially only to a defined target. Thus a protein-specific binding agent binds substantially only the defined protein, or to a specific region within the protein. As used herein, the term “[X] specific binding agent,” where [X] refers to a specific protein or peptide, includes anti-[X] antibodies (and functional fragments thereof) and other agents (such as aptamers or mirror-image aptamers) that bind substantially only to [X]. It is contemplated that [X] can be closely related proteins (for instance, isoforms of a protein, such as the short and long isoforms of SPATIAL) that are recognized by one specific binding agent.

Subject: Living multicellular, vertebrate organisms, a category which includes both human and veterinary subjects for example, mammals, rodents, and birds.

T cell(s) (or T lymphocyte): A lymphoid cell from the bone marrow that migrates to the thymus gland, where it develops into a mature differentiated T cell that circulates between blood and lymph. T cells are responsible for adaptive cell-mediated immunity. Adaptive cell-mediated immunity is immunity that confers resistance to pathogenic conditions (including, for example, neoplasia or infection by microbes, viruses, or bacteria) that are not susceptible to the innate immune response (for example, not susceptible to the antibody-making cells of the immune system). T cells generally cannot recognize foreign antigens without the help of antigen processing cells (APC), such as macrophages, dendritic cells or B cells. However, once the APC has helped T cells identify an antigen as “non-self,” T cells dominate the specific immune response directing macrophages, B cells, and other T cells in the body's defense. T cells also play a major role in graft rejection, graft versus host disease, some hypersensitivity reactions, and recognition and destruction of tumor cells because of the unique antigens some of these cells carry.

“Thymocytes” are developing T cells that are located in the thymus. Any particular thymocyte may be at one of several stages of development. Stages of thymocyte development may be distinguished by the expression of the surface protein markers called clusters of differentiation (CD). Expression of CD4 and CD8 markers are particularly useful for distinguishing various stages of thymocyte development. The least mature thymocytes do not express either CD4 or CD8 and are called “double negative” (or DN) cells. DN cells may also be represented as CD4⁻/CD8⁻. DN cells are found predominantly in the subcapsular and outer cortical regions of the thymus. DN cells may be further characterized as DN1, DN2, DN3 or DN4 thymocytes on the basis of other phenotypic markers, including for example CD25 and CD44. In particular, DN1 cells may be identified as CD25⁻/CD44⁺, DN2 cells may be identified as CD44⁺/CD25⁺, DN3 cells may be identified as CD25⁺/CD44⁻, and DN4 cells may be identified as CD44⁻/CD25⁻.

As thymocyte development progresses, thymocytes migrate into the cortex and begin to express both CD4 and CD8. CD4⁺/CD8⁺ thymocytes may be called “double positive” (or DP) cells. DP cells become responsive to antigens and are subject to positive and negative selection. Cells that successfully undergo selection then mature into CD4⁺/CD8⁻ or CD4⁻/CD8⁺ cells, which are also called “single positive” (or SP) cells. Single positive cells enter the thymic medulla and then leave the thymus, as mature T cells, to populate the peripheral lymphoid tissues.

“Naive T cells” are the end result of the thymocyte differentiation. Naive T cells leave the thymus and circulate in the peripheral blood and lymph system(s). Naive T cells comprise a pool of T cells that can recognize non-self antigens, but which have not yet encountered cognate antigen. The pool of naive T cells is required to mediate an acquired immune response to foreign antigens that the immune system has not previously processed. The naive T cells' activation and proliferation create an acquired immune response to the newly encountered foreign antigens.

“Donor T cells” are T cells the lineage of which can be traced to the donor of transplanted cells in a bone marrow transplant.

T cell deficiency: A state of having less than the number of at least one subset of T cells (for instance CD4-positive and/or CD8-positive T cells) considered to be normal for the particular species to which a subject belongs. This term is intended to encompass decreases in numbers of totals T cells and alterations in the ratios of T cell subtypes. T cell deficiency may occur as a result of normal, non-pathological physiological processes, such as T cell deficiency that occurs chronically over a long period of times as a result of natural aging processes (also referred to as age-related immunodeficiency). T cell deficiency also can occur as a direct or indirect result of pathological (or disease) processes. As used herein, “disease-related T cell deficiency” is a T cell deficiency that results as a direct or indirect consequence of a recognized pathological condition, not including, for example, natural aging. Indirect consequences of a pathological condition include, for example, treatments for a pathological condition that result in T cell deficiency. Some non-limiting examples of T cell deficiency that occurs as a direct consequence of a pathological condition are immunodeficiency caused by infection with at virus (such as HIV) or a genetic disorder (such as SCID).

Therapeutically effective amount: A quantity of a specified agent sufficient to achieve a desired effect in a subject being treated with that agent. For example, this may be the amount of an inhibitor of SPATIAL gene expression or SPATIAL protein activity necessary to measurably increase thymocyte number in a subject. In another example, a therapeutically effective amount of an agent may be the amount of the agent necessary to interfere with an interaction between SPATIAL and Uba3 and thereby increase thymocyte number in a subject. Ideally, a therapeutically effective amount of an agent is an amount sufficient to inhibit SPATIAL activity or to interfere with an interaction between SPATIAL and Uba3 without causing a substantial cytotoxic effect in the subject. The effective amount of an agent useful for inhibiting SPATIAL activity or to interfere with an interaction between SPATIAL and Uba3 will be dependent on the subject being treated, the severity of the affliction, and the manner of administration of the therapeutic composition.

An effective amount of an agent useful for inhibiting SPATIAL activity or for interfering with an interaction between SPATIAL and Uba3 may be administered in a single dose, or in several doses, for example daily, during a course of treatment. However, the frequency of administration is dependent on the preparation applied, the subject being treated, the severity and type of the affliction, and the manner of administration of the compound.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means including A, or B, or A and B. It is further to be understood that all base sizes (lengths) or amino acid sizes (lengths), and all molecular weight or molecular mass values, given for nucleic acids or polypeptides are approximate, and are provided for description. Although methods and materials similar or equivalent to those described herein can be used in the practice or testing of the embodiments of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Except as otherwise noted, the methods and techniques of the present disclosure are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

### III. Isolation and Preliminary Characterization of SPATIAL

The isolation and preliminary characterization of two SPATIAL nucleic acids (SEQ ID NOs: 1 and 3) from the mouse thymus, and the long and short protein isoforms encoded thereby (SEQ ID NOs: 2 and 4) are described in Examples 1 and 2 and in Flomerfelt et al., Genes and Immunity, 1:391-401, 2000 (Flomerfelt et al., 2000), which is specifically incorporated herein in its entirety. Unless expressly indicated (or the context requires) otherwise, the term “SPATIAL” throughout this specification is intended to refer to either (or both) of the long and/or short SPATIAL isoform(s) and/or the nucleic acid(s) encoding such isoform(s).

Subsequent to Flomerfelt et al., 2000, SPATIAL heterozygote and SPATIAL null mice were generated, as described in Example 3. It is widely believed that many vertebrates, including, for example, humans, mice, rats and chickens, experience an age-related decrease in the number of thymocytes (e.g., Aspinall and Andrew, J. Clin. Immunol., 20(4):250-256, 2000). This age-related effect is diminished in SPATIAL heterozygote and SPATIAL null mice, in which an increase in absolute thymocyte numbers and, in particular, in early stage thymocytes (for example, DN1 and DN2 thymocytes) was observed from ages 5 to 12 months, as compared to wild type littermates (Flomerfelt and Gress, Biol. Blood Marrow Transplant., 8(2):68, 2002). These results suggested that only after many months a complete inactivation of SPATIAL gene expression (SPATIAL null mouse) or partial inactivation of SPATIAL gene expression (SPATIAL heterozygote mouse) may affect thymocyte number in normal, aging mice.

It was, therefore, completely unexpected to find substantial thymic reconstitution within 3 weeks of bone marrow transplant in severely T cell deficient mice (for example, in Rag2 null mice) in which the SPATIAL gene was completely or partially inactivated. Moreover, continued characterization of SPATIAL protein isoforms has revealed that both isoforms are potent cell cycle inhibitors and expression of SPATIAL causes a wide variety of cell types to be arrested in the G1 phase. SPATIAL has also newly been found to interact with Uba3, which is a protein required for initiating the Nedd8 pathway which, in turn, is required for ubiquitin-mediated degradation of several cell cycle control proteins (e.g., Morimoto et al., Biochem. Biophys. Res. Commun., 270(3):1093-1096, 2000; Read et al., Mol Cell Biol., 20(7):2326-2333, 2000; Tateishi et al., J. Cell Biol., 155(4): 571-579, 2001). Although not bound by theory, it is believed that an interaction between SPATIAL and Uba3 prohibits Uba3 from initiating the Nedd8 pathway, which stops the degradation of critical cell cycle control proteins (such as p27kip1) and the cell cycle is blocked.

Specific aspects related to these discoveries are discussed more fully below.

### IV. Immune Deficiency

Inhibition of SPATIAL activity has been found to improve immune function in immune compromised subjects. Thus, methods of inhibiting SPATIAL to improve immune function in such subjects are now enabled. In particular examples, inhibition of SPATIAL activity results in increased numbers of thymocytes, which populate the immune system and thereby improve immune function. Hence, some method embodiments are particularly applicable for T cell deficient subjects.

In some methods, the subject has T cell deficiency that is directly or indirectly associated with a recognized pathological condition, not including, for example normal aging. A subject is T cell deficient when the subject has less than the number of T cells considered normal for the particular species to which the subject belongs. AT cell deficient subject may have, for example, at least 10% fewer, at least 25% fewer, at least 50% fewer, at least 75% fewer, at least 90% fewer T cells than normal T cell values for the species.

In a particular example, the normal T cell count in humans is generally considered to be in the range of about 650 to about 2010 CD3⁺ cells/μl blood, or about 350 to about 1260 CD4⁺ cells/μl Such normal ranges may depend upon the laboratory where the T cell count is performed, but one of skill in the art will appreciate how to determine a normal T cell count in particular circumstances. Non-limiting examples of T cell deficiency in a human include T cell counts less than a lower boundary of a normal range, for example, a T cell count of less than about 600, about 500, about 400, or about 300 CD3⁺ cells/μl blood, or in other examples, a T cell count of less than about 320, about 300, about 275, or about 250 CD4⁺ cells/μl blood.

T cell deficiency may also be determined functionally by the occurrence of physical findings or symptoms known in the art to be indicative of T cell deficiency, such as opportunistic infection. In other examples, the subject may fail to respond to vaccines, exhibit skewed ratios of T cells (for instance, increased CD8-positive cells, decreased CD-4 positive cells or visa versa). Such functional determinations of T cell deficiency are well within the knowledge of those of ordinary skill in the art.

T cell deficiency may arise as a consequence of numerous known diseases, including, without limitation, HIV infection, acquired immunodeficiency syndrome (AIDS), autoimmune disease, thymic hypoplasia, chronic mucocutaneous candidiasis, severe combined immunodeficiency (SCID), cellular immunodeficiency with immunoglobulins (Nezlof syndrome), immunodeficiency with thrombocytopenia and eczema (Wiskott-Aldrich syndrome), ataxia-telangiectasia, immunodeficiency with short-limbed dwarfism, immunodeficiency with thymoma, transcobalamin II deficiency, episodic lymphopenia with lymphotoxin, and idiopathic CD4 lymphocytopenia.

In other examples, T cell deficiency may be an indirect result of a disease state; for example, as a result of a medical treatment for a particular disease. For instance, chemotherapy treatment for many types of cancer can be cytotoxic to the chemotherapy patient's T cells; in which event, T cells are destroyed and the patient can become T cell deficient. Similarly, a subject given radiation treatment, which can be cytotoxic to T cells, may become T cell deficient as a result of the treatment. In other examples, steroid therapy or cytotoxic therapy (such as in the treatment of multiple sclerosis, autoimmune disease or rheumatoid arthritis) may result in T cell deficiency that is an indirect result of a disease state.

In some examples, the onset of T cell deficiency is relatively rapid (referred to as acute T cell deficiency). Rapid onset T cell deficiency may occur, for example, following chemotherapy or radiation therapy, or during some stages of HIV infection. In other examples, the onset of T cell deficiency is related to the onset, development or progression of disease, such as is the case of a genetic disorder affecting T cell development (such as, SCID).

T cell deficient subjects are readily identified by physical findings and diagnostic procedures that are well known in the art. All such methods of identification are contemplated by this disclosure. In some cases, T cell deficiency is a known outcome of a medical treatment, such as chemotherapy and/or radiation therapy, and recipients of such treatment are simply identified. In other cases, T cell deficient subjects present with recurrent serious infections, especially with opportunistic organisms. Physical examination of a subject may reveal failure to thrive, weight loss, enlargement or absence of lymph nodes, organomegaly, dermatitis, petechiae, facial abnormalities, cardiac abnormalities, oral candidiasis, dwarfism, short stature, digital clubbing, ataxia, telangiectasia, or listlessness.

Useful procedures for diagnosis of T cell deficiency include, for example, (i) complete blood count, absolute lymphocyte count, and morphologic review of blood-borne lymphocytes (such as, by FACS analysis); (ii) computerized tomography (CT) scan to delineate a small thymus gland; (iii) delayed hypersensitivity skin tests to recall antigens; (iv) measurement of T cell surface markers in peripheral blood cells; and (v) cellular functional assays, such as lymphocyte proliferation assays in response to mitogens, antigens and allogeneic cells. In some subjects, it may be helpful to evaluate the ability T cells to secrete cytokines. In many instances, a simple blood test to determine T cell count is all that is necessary. Trec analysis may be useful for determining T cell levels, especially levels of naive T cells (e.g., Douek, Vaccine, 18(16):249-256, 2000)

### IV. Inhibition of SPATIAL Activity

This disclosure reveals that SPATIAL activity includes, without limitation, (i) suppression of cell growth, at least in part, by prohibiting cells' entry into the proliferative stages of the cell cycle; (ii) suppression of thymocyte development in vivo; thus, decreasing or negatively regulating thymocyte numbers, and (iii) formation of a protein-protein interaction with Uba3. Inhibition of any SPATIAL activity, including the foregoing examples, that has a specified result is contemplated herein.

SPATIAL activity may be inhibited at any point in the progression from activation of transcription of the SPATIAL gene, transcription of the SPATIAL gene, post-transcriptional message processing, translation of SPATIAL mRNA(s), post-translational protein processing, to actual protein activity. Moreover, any agent capable of inhibiting a SPATIAL activity is contemplated by this disclosure, such agents may include for example, small molecules, drugs, chemicals, compounds, siRNA, ribozymes, anti-sense oligonucleotides, SPATIAL inhibitory antibodies, SPATIAL inhibitory peptides (such as, Uba3 peptides or SPATIAL peptide fragments), aptamers, or mirror-image aptamers.

### 1. Inhibition of SPATIAL Nucleic Acids

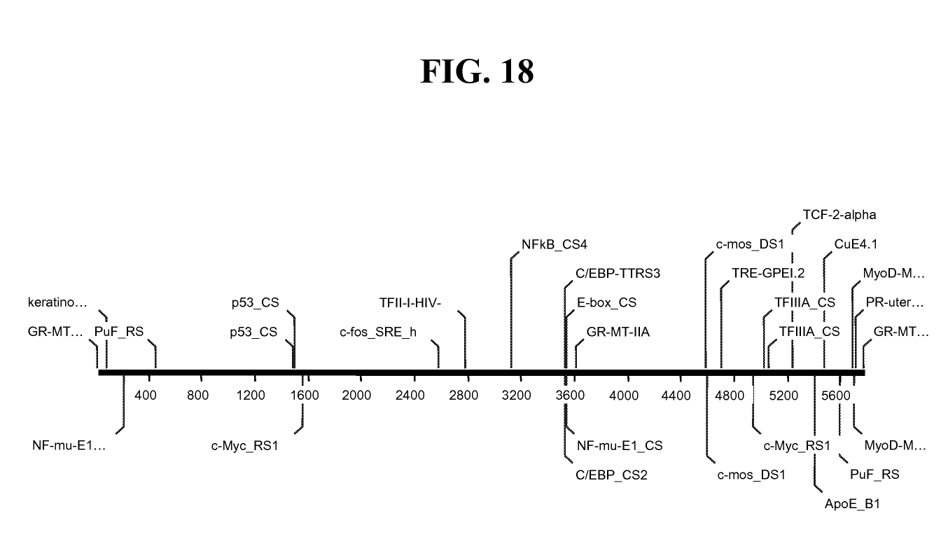

A regulatory region of the SPATIAL gene has been isolated and its nucleic acid sequence is set forth in SEQ ID NO: 7. With this information, it is now possible block or inhibit activation or repression of the SPATIAL gene; for example, by targeting or manipulating trans-acting activators or silencers of the SPATIAL gene. FIG. 18 and Table 1 identify consensus and known binding sites for selected trans-acting factors present in the SPATIAL gene regulatory region. These data were derived by computer analysis of the SPATIAL promoter sequence using the TFDSites transcription factor database (Ghosh, Nucleic Acids Res., 28:308-310, 2000).

TABLE 1

Selected Transcription Factor Binding Sites

| | | | | |
|---|---|---|---|---|
| Site Name | Position¹ | Strand | Sequence | Reference |

| | | | | |
|---|---|---|---|---|
| ApoE B1 | 5406 | + | GCCCACCTC | J. Biol. Chem., 263:8300-8308, 1988 |
| c-fos SRE h | 2572 | − | GATGTCC | Mol. Cell. Biol., 7:1217-1225, 1987 |
| c-mos DS1 | 4579 | − | TGGTTTG | J. Mol. Biol., 193:255-266, 1987 |
| c-mos DS1 | 4600 | + | TGGTTTG | J. Mol. Biol., 193:255-266, 1987 |
| c-Myc_RS1 | 4937 | + | TCTCTTA | EMBO J., 8:4273, 1989 |
| C/EBP-TTRS3 | 3528 | − | TCTTACTC | Proc. Natl. Acad. Sci., 85:3840-3844, |
| | | | | 1988 |
| C/EBP CS2 | 3528 | − | TCTTACTC | Proc. Natl. Acad. Sci, 85:3840-3844, |
| | | | | 1988 |
| CuE4.1 | 5481 | + | CAGGTGGT | Science, 227:134-40, 1985 |
| E-box CS | 3536 | − | CAGGTGGC | Science, 227:134-40, 1985 |
| GR-MT-IIA | 6 | + | TGTCCT | Nature, 308:513-519, 1984 |
| GR-MT-IIA | 3613 | + | TGTCCT | Nature, 308:513-519, 1984 |
| GR-MT-IIA | 5776 | − | TGTCCT | Nature, 308:513-519, 1984 |
| keratinocyt | 84 | − | AAGCCAAA | J. Biol. Chem., 268:377-384, 1993 |
| MyoD-MCK-le | 5692 | + | CACGTG | Science, 255:979-983, 1992 |
| MyoD-MCK-le | 5697 | − | CACGTG | Science, 255:979-983, 1992 |
| NF-mu-E1 CS | 210 | + | CAGCTGGC | Genetika, 26:804-816, 1990 |
| NF-mu-E1 CS | 3536 | − | CAGGTGGC | Genetika, 26:804-816, 1990 |
| NFkB CS4 | 3125 | + | GGGACTTTC | Hamatol. Bluttransfus., 32:411-415, |
| | | | | 1989 |
| p53 CS | 1485 | + | GAGCAAGCCC | Nature Genet., 1:45-49, 1992 |
| p53 CS | 1494 | − | GGGCTTGCTC | Nature Genet., 1:45-49, 1992 |
| PR-uterogl.2 | 5708 | − | AGTCCTTT | Nucleic Acids Res., 15:4535-4552, |
| | | | | 1987 |
| PuF RS | 449 | − | GGGTGGG | Mol. Cell. Biol., 9:5123-5133, 1989 |
| PuF RS | 5596 | − | GGGTGGG | Mol. Cell. Biol, 9:5123-5133, 1989 |
| TCF-2-alpha | 5243 | + | GAGGAAGC | Nucleic Acids Res., 20:3-26, 1992 |
| TFII-I-HIV-1-I | 2775 | + | GTCTCTCT | Nature, 354:245-248, 1991 |
| nr1 | | | | |
| TFIIIA CS | 5021 | + | CGGGCTGGAG | Genetika, 26:804-16, 1990 |
| TFIIIA CS | 5061 | + | CAGGATAGAA | Genetika, 26:804-16, 1990 |
| TRE-GPEI.2 | 4705 | + | TGATTCAG | EMBO J., 9:1131, 1990 |

¹Nucleotide positions correspond to those set forth in SEQ ID NO: 7

In specific examples, trans-acting activators may be prohibited from binding to their cis-acting elements in the SPATIAL regulatory sequence, or the binding of trans-acting silencers to their cognate sites in the SPATIAL regulatory sequence may be promoted or enhanced; in either event, resulting in suppression or inhibition of SPATIAL gene expression. Alternatively, transcription of the SPATIAL gene may be completely or partially inhibited by specific silencing of the gene by DNA methylation (see, for example, U.S. Pal. No. 5,840,497), by inhibition of the nuclear enzyme histone deacetylase (see, for example, U.S. Pal. No. 6,495,719), or through the use of gene promoter-suppressing nucleic acids (such as, Utrons) as described in U.S. Pal. No. 6,022,863.

It is noted that the SPATIAL regulatory region directs cell-specific expression of the SPATIAL gene in a limited subset of tissues, including the thymus and testes. Thus, the SPATIAL regulatory region may be used to direct tissue-specific expression of heterologous transcriptional units operably linked to all or part of the SPATIAL regulatory region. For example, the SPATIAL regulatory region may be used to direct thymus-specific expression of SPATIAL inhibitory agents, such as polypeptides or peptides that inhibit a SPATIAL activity, including in some specific examples Uba3 inhibitory peptides. In other examples, the SPATIAL regulatory region may be used to specifically direct expression of other heterologous transcriptional units that may have desired effects in thymus, such as growth factors or other trophic factors. A “heterologous transcriptional unit” is a transcribable nucleic acid sequence that is not normally (for example, in the genome) found adjacent to a second nucleic acid sequence. A first nucleic acid sequence is “operably linked” with a second nucleic acid sequence when the first nucleic acid sequence is in a functional relationship with the second nucleic acid sequence; for instance, a regulatory region is operably linked to a coding sequence if the regulatory region affects the transcription or expression of the coding sequence.

SPATIAL gene expression may also be inhibited by interfering with SPATIAL mRNA transcription, processing or translation, for example, using siRNA, ribozymes or anti-sense oligonucleotides, as described in the following subsections.

a. siRNA

Expression of SPATIAL can be reduced using small inhibitory RNAs, for instance using techniques similar to those described previously (see, e.g., Tuschl et al., Genes Dev., 13:3191-3197, 1999; Caplen et al., Proc. Natl. Acad. Sci., 98, 9742-9747, 2001; and Elbashir et al., Nature, 411:494-498, 2001).

siRNAs can induce gene-specific inhibition of expression in invertebrate and vertebrate species. These RNAs are suitable for interference or inhibition of expression of a target gene and comprise double stranded RNAs of about 15 to about 40 nucleotides containing a 3′ and/or 5′ overhang on each strand having a length of 0 to about 5 nucleotides, wherein the sequence of the double stranded RNAs is substantially identical to a portion of a mRNA or transcript of the target gene for which interference or inhibition of expression is desired, such as the SPATIAL mRNA. The double stranded RNAs can be formed from complementary ssRNAs or from a single stranded RNA that forms a hairpin or from expression from a DNA vector. In some examples, an siRNA sequence has ˜50% G or C nucleotides, no homology in the sequence database to genes other than the intended target and no run of identical nucleotides

In addition to native RNA molecules, RNAs suitable for inhibiting or interfering with the expression of a target sequence include RNA derivatives and analogs. For example, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′-O-alkylated residues or 2′-deoxy-2′-halogenated derivatives. Particular examples of such carbohydrate moieties include 2′-O-methyl ribosyl derivatives and 2′-O-fluoro ribosyl derivatives.

The RNA bases may also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

b. Ribozymes

Also contemplated herein are ribozymes, which are gene-targeting agents useful for specific inhibition of gene expression (see, e.g., Zamecnik and Stephenson, Proc. Natl. Acad. Sci., 75:280-284, 1978; Altman, Proc. Natl. Acad. Sci., 90:10898-10900, 1993; Rossi, Chem. Biol., 6:R33-R37, 1999; Trang et al., Proc. Natl. Acad. Sci., 97:5812-5817, 2000), such as inhibition of SPATIAL gene expression.

The production and use of ribozymes are disclosed in U.S. Pal. No. 4,987,071 to Cech and U.S. Pal. No. 5,543,508 to Haselhoff. Further, RNA enzymes capable of cleaving specific substrate RNA are known in the art, including, for instance, the hairpin (Hampel et al., Nucleic Acids Res., 18:299-304, 1990; Yu et al., Proc. Natl. Acad. Sci., 90:6340-6344, 1993), the hammerhead (Forster and Symons, Cell, 50:9-16, 1987; Uhlenbeck, Nature, 328:596-600, 1987; Cantor et al., Proc. Natl. Acad. Sci., 90:10932-10936, 1993), the axehead (Branch and Robertson, Proc. Natl. Acad. Sci., 88:10163-10167, 1991), the group I intron (Hampel et al., Nucleic Acids Res., 18:299-304, 1990), and RNase P (Yuan et al., Proc. Natl. Acad. Sci., 89:8006-8010, 1992).

The substrate-binding region of RNA enzymes may be modified, using methods well known in the art, to be complementary to a portion of a target RNA, such as SPATIAL mRNA(s). When delivered to cells expressing the target RNA, the RNA enzyme will then form a complex with and cleave the target RNA. The target-specific ribozyme may then dissociate from the cleaved substrate RNA, and repeatedly hybridize to and cleave additional substrate RNA molecules; ultimately, inhibiting the expression and activity of any protein encoded by the target RNA.

The nucleic acid sequences of both the long and short isoforms of SPATIAL mRNA are known (SEQ ID NO: 1 and 3, wherein T equals U). Thus, a ribozyme useful for specifically cleaving SPATIAL mRNA may be designed by selecting, for example, at least 5, at least 10, at least 15, at least 20, at least 30 consecutive nucleotides of SPATIAL mRNA(s) as a substrate for SPATIAL-specific ribozyme cleavage.

c. Anti-Sense Oligonucleotides

The methods disclosed herein further contemplate a reduction of SPATIAL activity in vitro or in vivo by introducing into cells an anti-sense construct based on the SPATIAL-encoding sequence, including the cDNA sequences of either the short or long thymic isoforms of SPATIAL (SEQ ID NOs: 1 and 3, respectively) or flanking regions thereof. For anti-sense suppression, a nucleotide sequence from a SPATIAL-encoding sequence, for example all or a portion of the cDNA of the long or short SPATIAL isoform, is arranged in reverse orientation relative to the promoter sequence in the transformation vector. Specific embodiments of anti-sense oligonucleotides are discussed in Example 13.

The introduced sequence need not be a full-length SPATIAL cDNA or gene or reverse complement thereof, and need not be exactly homologous to the equivalent sequence found in the cell type to be transformed. Generally, however, where the introduced sequence is of shorter length, a higher degree of homology to the native SPATIAL sequence will be needed for effective anti-sense suppression. The introduced anti-sense sequence in the vector may be at least 30 nucleotides in length, and improved anti-sense suppression will typically be observed as the length of the anti-sense sequence increases. The length of the anti-sense sequence in the vector advantageously may be greater than 100 nucleotides. For suppression of the SPATIAL gene itself, transcription of an anti-sense construct results in the production of RNA molecules that are the reverse complement of mRNA molecules transcribed from the endogenous SPATIAL gene in the cell.

Although the exact mechanism by which anti-sense RNA molecules interfere with gene expression has not been elucidated, it is believed that anti-sense RNA molecules bind to the endogenous mRNA molecules and thereby inhibit translation of the endogenous mRNA.

d. Oligonucleotide Synthesis

Oligonucleotides, such as single-stranded DNA or RNA oligonucleotides, including, for example, aptamers or anti-sense oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well-known, this reaction can be prevented selectively, where desired, by removing the 5′ phosphates of the other polynucleotide(s) prior to ligation.

### 2. Inhibition of SPATIAL Polypeptide(s)

Certain methods disclosed herein contemplate inhibition of SPATIAL polypeptides by, for example, SPATIAL inhibitory antibodies, SPATIAL inhibitory peptides (such as, Uba3 peptides or SPATIAL peptide fragments), aptamers or mirror-image aptamers.

a. Inhibitory Antibodies

Antibodies that inhibit SPATIAL activity may be monoclonal or polyclonal; though, monoclonal inhibitory antibodies are preferred. Monoclonal or polyclonal antibodies may be produced to specifically recognize and bind to either of the SPATIAL isoforms (SEQ ID NOs: 2 or 4) or fragments thereof as a first step in producing SPATIAL inhibitory antibodies. Optimally, antibodies raised against these proteins or peptides would specifically detect the protein or peptide with which the antibodies are generated. That is, an antibody generated to a SPATIAL isoform or a fragment thereof would recognize and bind one or both SPATIAL isoforms and would not substantially recognize or bind to other proteins found in target cells.

The determination that an antibody specifically detects a SPATIAL isoform is made by any one of a number of standard immunoassay methods; for instance, the Western blotting technique (Sambrook et al., Molecular Cloning: A Laboratory Manual, New York: Cold Spring Harbor Laboratory Press, 1989). To determine that a given antibody preparation (such as one produced in a mouse) specifically detects one or more SPATIAL isoforms by Western blotting, total cellular protein is extracted from human cells (for example, thymic stromal cells) and electrophoresed on a sodium dodecyl sulfate-polyacrylamide gel. The proteins are then transferred to a membrane (for example, nitrocellulose) by Western blotting, and the antibody preparation is incubated with the membrane. After washing the membrane to remove non-specifically bound antibodies, the presence of specifically bound antibodies is detected by the use of an anti-mouse antibody conjugated to an enzyme such as alkaline phosphatase. Application of an alkaline phosphatase substrate 5-bromo-4-chloro-3-indolyl phosphate/nitro blue tetrazolium results in the production of a dense blue compound by immunolocalized alkaline phosphatase. Antibodies that specifically detect a SPATIAL isoform will, by this technique, be shown to bind to the protein band(s) corresponding to the apparent molecular weight(s) of one or both SPATIAL isoforms. Non-specific binding of the antibody to other proteins may occur and may be detectable as a weak signal on the Western blot. The non-specific nature of this binding will be recognized by one skilled in the art by the weak signal obtained on the Western blot relative to the strong primary signal arising from the specific antibody-SPATIAL protein binding.

Substantially pure SPATIAL protein or protein fragment (peptide) suitable for use as an immunogen may be isolated from the transfected or transformed cells as described below. Monoclonal or polyclonal antibody to the protein can then be prepared, for example, using any of the detailed procedures described in Harlow and Lane (Antibodies, A Laboratory Manual, New York: Cold Spring Harbor Laboratory Press, 1988).

In specific examples, monoclonal antibody to an epitope of the SPATIAL protein identified can be prepared from murine hybridomas according to the classical method of Kohler and Milstein (Nature, 256:495-497, 1975) or derivative methods thereof. Briefly, a mouse is repetitively inoculated with a few micrograms of the selected protein immunogen (for example, SPATIAL protein, SPATIAL protein fragment, or SPATIAL synthetic peptide) over a period of a few weeks. The mouse is then sacrificed, and the antibody-producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall (Meth. Enzymol., 70:419-439, 1980), and derivative methods thereof. Selected positive clones can be expanded and their monoclonal antibody product harvested for use.

Effective antibody production (whether monoclonal or polyclonal) is affected by many factors related both to the antigen and the host species. For example, small molecules tend to be less immunogenic than others and may require the use of carriers and adjuvant. Also, host animals vary in response to site of inoculations and dose, with either inadequate or excessive doses of antigen resulting in low titer antisera. Small doses (ng level) of antigen administered at multiple intradermal sites appear to be most reliable. An effective immunization protocol for rabbits can be found in Vaitukaitis et al. (J. Clin. Endocrinol Metab., 33:988-991, 1971).

Booster injections can be given at regular intervals, and antiserum or spleen cells, as applicable, harvested when antibody titer thereof, as determined semi-quantitatively, for example, by double immunodiffusion in agar against known concentrations of the antigen, begins to fall. See, for example, Ouchterlony et al (In Handbook of Experimental Immunology, ed. by Wier, D., Ch. 19, Blackwell, 1973). Plateau concentration of antibody is usually in the range of about 0.1 to 0.2 mg/ml of serum (about 12 μM). Affinity of polyclonal antisera for the antigen can be determined by preparing competitive binding curves, as described, for example, by Fisher (Manual of Clinical Immunology, Ch. 42, 1980).

Antibodies may be screened for SPATIAL inhibitory activity, such as the ability to disrupt the protein-protein interaction between SPATIAL and Uba3, or block SPATIAL-mediated suppression of cell growth or increase thymocyte number in vivo. Specific screening methods for agents that inhibit a SPATIAL activity, such as a SPATIAL inhibitory antibody, are described in Examples 18 and 19 or elsewhere in this disclosure.

For administration to human subjects, antibodies, for example, SPATIAL-specific monoclonal antibodies, can be humanized by methods known in the art. Antibodies with a desired binding specificity can be commercially humanized (Scotgene, Scotland, UK; Oxford Molecular, Palo Alto, Calif.).

b. Inhibitory Peptides

Some method embodiments disclosed herein contemplate polypeptide or peptide agents that measurably reduce at least one biological activity of SPATIAL, for example peptides that can inhibit a SPATIAL activity. Inhibitory peptides are typically less than about 250 amino acid residues in length, for example, less than about 200 amino acid residues, less than about 150 amino acid residues, less than about 100 amino acid residues, less than about 75 amino acid residues, less than about 50 amino acid residues, less than about 40 amino acid residues, or less than about 30 amino acid residues in length.

In some embodiments, inhibitory peptides are fragments of the SPATIAL polypeptide.

In particular examples, SPATIAL inhibitory peptides interfere with a protein-protein interaction between SPATIAL and Uba3. As described in more detail in Examples 9-12, SPATIAL and Uba3 are involved in a direct protein-protein interaction. It is believed that this interaction is involved in SPATIAL-mediated suppression of cell growth, at least in part, because co-expression of Uba3 with SPATIAL overcomes such growth suppression. Specific regions of Uba3 that are involved in a protein-protein interaction with SPATIAL are disclosed in Example 10.

In some embodiments, Uba3 polypeptides and fragments and variants thereof are useful as agents to interfere with a SPATIAL/Uba3 interaction. For example, Uba3 peptides, which include one or more regions that interact with SPATIAL, can be useful for interfering with SPATIAL binding to Uba3, and thereby interfering with SPATIAL functions mediated through Uba3 binding, such as growth suppression.

In other embodiments, SPATIAL peptides and variants thereof are useful as agents to interfere with a SPATIAL/Uba3 interaction. For example, SPATIAL peptides may compete with endogenous or full-length SPATIAL for binding to Uba3, and thereby interfering with SPATIAL binding to Uba3. Particularly useful SPATIAL peptides and variants interfere with a SPATIAL/Uba3 interaction, but do not also block Uba3 biological activity.

Uba3 peptides useful for the disclosed methods may be at least 15, at least 20, at least 30, at least 40, at least 50 or even more consecutive amino acids of Uba3 (SEQ ID NO: 6). More particularly, an Uba3 peptide may be at least 15, at least 20, at least 30, at least 40, or at least 50 consecutive Uba3 amino acids corresponding to residues 183-308, residues 190-300, residues 200-290, residues 210-280, or residues 220-270 of SEQ ID NO: 6.

SPATIAL peptides useful the disclosed methods may be at least 15, at least 20, at least 30, at least 40, at least 50 or even more consecutive amino acids of either isoform of SPATIAL (SEQ ID NOs: 2 or 4).

c. Specific-Binding Oligonucleotides (Aptamers and Mirror-Image Aptamers)

Specific-binding oligonucleotides (such as, aptamers and mirror-image aptamers (a.k.a., Spiegelmers™)) are oligonucleotides with high affinity and high specificity for a wide variety of target molecules (as reviewed in Jayasena, Clin. Chem., 45(9):1628-1650, 1999), including, for example, polypeptides, peptides, metal ions, organic dyes, drugs, amino acids, cofactors, nucleotides, antibiotics, nucleotide base analogs, and aminoglycosides. In particular examples, a specific-binding oligonucleotide binds to a SPATIAL isoform and inhibits its activity. In other examples, a specific binding oligonucleotide disrupts an interaction between SPATIAL and Uba3.

Specific-binding oligonucleotides for a particular target are typically selected from a large “library” of unique nucleic acid molecules (often as many as 10¹⁴-10¹⁵ different compounds or more). Each oligonucleotide molecule in the library contains a unique nucleotide sequence that can, in principle, adopt a unique three-dimensional shape. The target-specific oligonucleotides are thought to present a surface that is complementary to the target molecule.

Chemically modified oligonucleotides may be included in oligonucleotide libraries, for example, 2,6-diaminopyrimidine, xanthine, 2,4-difluorotoluene, 6-methylpurine, 5-(1-pentynyl-2-deoxyuridine), pyrimidines modified with 2′-NH₂ and 2′-F functional groups.

The library of nucleotide sequences is exposed to the target (such as, a protein, small molecule, or supramolecular structure) and allowed to incubate for a period of time. Where a mirror-image aptamer (commonly known as a Spiegelmer) is the desired product, the oligonucleotide library is exposed to an enantiomeric form of the natural target. The molecules in the library with weak or no affinity for the target will, on average, remain free in solution while those with some capacity to bind will tend to associate with the target. The specific oligonucleotide/target complexes are then separated from the unbound molecules in the mixture by any of several methods known in the art. Target-bound oligonucleotides are separated and amplified using common molecular biology techniques to generate a new library of oligonucleotide molecules that is substantially enriched for those that can bind to the target. The enriched library is used to initiate a new cycle of selection, partitioning and amplification.

After several cycles (such as, 5-15 cycles) of the complete process, the library of oligonucleotide molecules is reduced from 10¹⁴-10¹⁵ or more unique sequences to a small number that bind tightly to the target of interest. Individual oligonucleotide molecules in the mixture are then isolated, and their nucleotide sequences are determined. In most cases, isolated target-specific oligonucleotides are further refined to eliminate any nucleotides that do not contribute to target binding or oligonucleotide structure. Target-specific oligonucleotides (referred to as aptamers) truncated to their core binding domain typically range in length from 15 to 60 nucleotides.

Once a sequence is identified, the target-specific oligonucleotide may be prepared by any known method, including synthetic, recombinant, and purification methods. Any one target-specific oligonucleotide may be used alone or in combination with other oligonucleotides specific for the same target. Where an enantiomeric form of the target was combined with the library, as discussed above, the L-form of the isolated oligonucleotide sequence(s) is synthesized to generate a mirror-image aptamer, which is specific for the naturally occurring target. Representative methods of making aptamers specific for non-DNA-binding proteins are described, for example, in U.S. Pat. No. 5,840,867, and in Jayasena, Clin. Chem., 45(9):1628-1650, 1999.

SPATIAL-specific aptamers or mirror-image aptamers may be screened for those that inhibit SPATIAL activity or interfere with an interaction between SPATIAL and Uba3, as described in additional detail below.

### V. Polypeptides, Peptides and Variants

The methods disclosed herein contemplate at least the use of SPATIAL inhibitory polypeptides and peptides (including, as one non-limiting example, Uba3 peptides capable of inhibiting a SPATIAL activity or interfering with a SPATIAL/Uba3 interaction), SPATIAL polypeptides, peptides, and variants thereof.

Methods of producing polypeptides, peptides and variants thereof, such as SPATIAL inhibitory peptides or SPATIAL polypeptides, are known in the art. For examples, peptides may be produced synthetically (see, e.g., Synthetic Peptides, A User's Guide, Second Edition, ed. by Gregory Grant, New York: Oxford University Press, 2002). In other examples, peptides may be produced by proteolytic digestion of isolated, full-length polypeptides. Numerous proteolytic enzymes useful for such purposes are known, including trypsin, papain, pepsin, and bromelain. In still other examples, in vitro translation may be used to produce proteins or peptides. Numerous commercially available kits are now available for in vitro translation reactions (see, for example, kits available from Ambion, Novagen, Amersham Pharmacia Biotech, Roche Molecular Biochemicals, Promega, Stratagene, and Sigma).

Polypeptide and peptide agents (such as, SPATIAL inhibitory peptides, including Uba3 peptides) or reagents (such as, SPATIAL immunogens for production of SPATIAL inhibitory antibodies) may also be produced in bacterial expression systems, such as E. coli, in large amounts for use in the disclosed methods. Methods and plasmid vectors for producing polypeptides and peptides in bacteria are described in Sambrook et al. (Molecular Cloning: A Laboratory Manual, New York: Cold Spring Harbor Laboratory Press, Ch. 17, 1989). Such proteins can be produced in bacteria by placing a strong, regulated promoter and an efficient ribosome-binding site upstream of the applicable transcriptional unit (for example, see SEQ ID NOs: 1 and 3 for the cDNA sequences of SPATIAL short and long isoforms, respectively, and see SEQ ID NO: 5 for the Uba3 cDNA sequence). If low levels of protein are produced, additional steps may be taken to increase protein production; if high levels of protein are produced, purification is relatively easy. Suitable methods are presented in Sambrook et al. (Molecular Cloning: A Laboratory Manual, New York: Cold Spring Harbor Laboratory Press, 1989) and are well known in the art. Often, proteins expressed at high levels are found in insoluble inclusion bodies. Methods for extracting proteins from these aggregates are described by Sambrook et al. (Molecular Cloning: A Laboratory Manual, Ch. 17, New York: Cold Spring Harbor Laboratory Press, 1989).

For expression of polypeptide and peptide agents in mammalian cells, a nucleic acid encoding the polypeptide or peptide may be ligated to heterologous promoters, such as the simian virus (SV) 40 promoter in the pSV2 vector (Mulligan and Berg, Proc. Natl. Acad. Sci. USA 78:2072-2076, 1981), and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet., 1:327-341, 1982) and mycophenolic acid (Mulligan and Berg, Proc. Natl. Acad. Sci., 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR. Such matters are well known to those of ordinary skill in the art.

Transfer of DNA into eukaryotic cells, in particular human or other mammalian cells, is now a conventional technique. The vectors are introduced into the recipient cells as pure DNA (transfection) by, for example, precipitation with calcium phosphate (Graham and vander Eb, Virology, 52:466, 1973) or strontium phosphate (Brash et al., Mol Cell Biol, 7:2013, 1987), electroporation (Neumann et al., EMBO J., 1:841, 1982), lipofection (Felgner et al., Proc. Natl. Acad. Sci., 84:7413, 1987), DEAE dextran (McCuthan et al., J. Natl. Cancer Inst., 41:351, 1968), microinjection (Mueller et al., Cell, 15:579, 1978), protoplast fusion (Schafner, Proc. Natl. Acad. Sci., 77:2163-2167, 1980), pellet guns (Klein et al., Nature, 327:70, 1987) or electroporation (Neumann et al., EMBO J., 1(7):841-845, 1982). Alternatively, the polypeptide- or peptide-encoding nucleic acid sequence(s), or fragments thereof, can be introduced by infection with virus vectors. Systems have been developed that use, for example, retroviruses (Bernstein et al., Gen. Engr'g, 7:235, 1985), adenoviruses (Ahmad et al., J. Virol, 57:267, 1986), Herpes virus (Spaete et al., Cell, 30:295, 1982) or lentivirus (Olsen, Somal. Cell. Mol. Genet., 26:131-45, 2001; Brenner and Malech, Biochim. Biophys. Acta, 1640(1):1-24, 2003).

Polypeptide- or peptide-encoding sequences can also be delivered to target cells in vitro via non-infectious systems, for instance liposomes.

### 1. Polypeptide or Peptide Variants and Nucleic Acids Encoding Such Variants

Variant polypeptides or peptides, such as SPATIAL variants, useful in the disclosed methods include proteins that differ in amino acid sequence from the disclosed sequences (such as, SPATIAL(S) in SEQ ID NO: 2, SPATIAL(L) in SEQ ID NO: 4, and Uba3 in SEQ ID NO: 6) but that share at least 50% amino acid sequence identity with the provided SPATIAL protein. Other variants will share at least 60%, at least 75%, at least 80%, at least 90%, at least 95%, or at least 98% amino acid sequence identity. Manipulation of the nucleotide sequence(s) encoding the subject polypeptide(s) or peptide(s) (for example, SPATIAL or Uba3 nucleic acid sequences) using standard procedures, including for instance site-directed mutagenesis or PCR, can be used to produce such variants. The simplest modifications involve the substitution of one or more amino acids for amino acids having similar biochemical properties. These so-called conservative substitutions are likely to have minimal impact on the activity of the resultant protein, so long as they do not affect amino acids in any active sites and/or binding pockets. Table 2 shows amino acids that may be substituted for an original amino acid in a protein, and which are regarded as conservative substitutions.

TABLE 2

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

More substantial changes in enzymatic function or other protein features may be obtained by selecting amino acid substitutions that are less conservative than those listed in Table 2. Such changes include changing residues that differ more significantly in their effect on maintaining polypeptide backbone structure (for example, sheet or helical conformation) near the substitution, charge, or hydrophobicity of the molecule at the target site, or bulk of a specific side chain. The following substitutions are generally expected to produce the greatest changes in protein properties: (i) a hydrophilic residue (for example, seryl or threonyl) is substituted for (or by) a hydrophobic residue (for example, leucyl, isoleucyl, phenylalanyl, valyl or alanyl); (ii) a cysteine or proline is substituted for (or by) any other residue; (iii) a residue having an electropositive side chain (for example, lysyl, arginyl, or histadyl) is substituted for (or by) an electronegative residue (for example, glutamyl or aspartyl); or (iv) a residue having a bulky side chain (for example, phenylalanine) is substituted for (or by) one lacking a side chain (for example, glycine).

Variant SPATIAL-encoding sequences may be produced by standard DNA mutagenesis techniques, for example, M13 primer mutagenesis. Details of these techniques are provided in Sambrook et al. (Molecular Cloning: A Laboratory Manual, New York: Cold Spring Harbor Laboratory Press, 1989, Ch. 15). By the use of such techniques, variants may be created which differ in minor ways from the SPATIAL or Uba3 sequences disclosed. DNA molecules and nucleotide sequences that are derivatives of those specifically disclosed herein, and which differ from those disclosed by the deletion, addition, or substitution of nucleotides while still encoding a protein that has at least 70% sequence identity with the disclosed SPATIAL (SEQ ID NOs: 1 or 3) or Uba3 sequences (SEQ ID NO: 5), are comprehended by this disclosure. Also comprehended are more closely related nucleic acid molecules that share at least 75%, at least 80%, at least 85%, at least 90%, at least 95%, or at least 98% or more nucleotide sequence identity with the disclosed SPATIAL sequences. In their most simple form, such variants may differ from the disclosed sequences by alteration of the coding region to fit the codon usage bias of the particular organism into which the molecule is to be introduced.

Alternatively, the coding region may be altered by taking advantage of the degeneracy of the genetic code to alter the coding sequence such that, while the nucleotide sequence is substantially altered, it nevertheless encodes a protein having an amino acid sequence substantially similar to the disclosed SPATIAL (SEQ ID NOs: 2 and 4) and/or Uba3 (SEQ ID NO: 6) protein sequences. Based upon the degeneracy of the genetic code, variant DNA molecules may be derived from the nucleic acid sequences disclosed herein using standard DNA mutagenesis techniques as described above, or by synthesis of DNA sequences.

Variants of the SPATIAL protein isoforms and/or variants of Uba3 may also be defined in terms of their sequence identity with the protein sequences shown in SEQ ID NOs: 2 and 4 (for SPATIAL) and SEQ ID NO: 6 (for Uba3). For instance, the disclosed methods contemplate the use of proteins that share at least 50%, at least 60%, at least 75%, at least 80%, at least 85%, at least 90%, at least 95%, or at least 98% or more amino acid sequence identity with a SPATIAL (SEQ ID NO: 2 or 4) or Uba3 (SEQ ID NO: 6) protein sequence disclosed herein. Nucleic acid sequences that encode such proteins may readily be determined simply by applying the genetic code to the amino acid sequence of a SPATIAL protein, and such nucleic acid molecules may readily be produced by assembling oligonucleotides corresponding to portions of the sequence.

### VI. Antiproliferative Uses of SPATIAL

SPATIAL has been shown herein to be a high-level and potent negative regulator of the cell cycle in a wide variety of cell types. For example, SPATIAL has been shown to effectively arrest cell cycle progression even in highly transformed cell lines, in which numerous cell cycle control pathways are deregulated. Thus, methods of using SPATIAL polypeptides, fragments, variants and/or mimetics for the treatment of hyperproliferative disorders are now available.

It is noted that because the disclosed SPATIAL cell cycle inhibitors are likely to be cytostatic rather than cytotoxic, these inhibitors may produce fewer, perhaps significantly fewer, of the unwanted side effects that often accompany treatments for hyperproliferative disorders. For example, undesirable side effects that accompany chemotherapies based on killing rapidly dividing cells, such as cancer cells, but also including certain normal cells, may be avoided or lessened.

Moreover, because the disclosed SPATIAL cell cycle inhibitors arrest cell growth rather than cause cell death, such inhibitors may be useful in combination therapies for treatment of hyperproliferative disorders. For example, a disclosed cell cycle inhibitor may be used to slow or stop cell division in a neoplasia, and thereby extend the life of a subject, while a vaccine directed to the neoplasia is co-administered in order to specifically target and remove the now-quiescent neoplasia (Ruffini et al., Biomed. Pharmacother., 56(3):129-132, 2002; Kwak, Semin. Oncol., 30(3 Suppl 8):17-22, 2003).

Neoplasias include any biological condition in which one or more cells have undergone characteristic anaplasia with loss of differentiation, increased rate of growth, invasion of surrounding tissue, and which is capable of metastasis. The resultant neoplasm is also known as cancer or a tumor. The term(s) includes breast carcinomas (e.g. lobular and duct carcinomas), and other solid tumors, sarcomas, and carcinomas of the lung like small cell carcinoma, large cell carcinoma, squamous carcinoma, and adenocarcinoma, mesothelioma of the lung, colorectal adenocarcinoma, stomach carcinoma, prostatic adenocarcinoma, ovarian carcinoma such as serous cystadenocarcinoma and mucinous cystadenocarcinoma, ovarian germ cell tumors, testicular carcinomas, and germ cell tumors, pancreatic adenocarcinoma, biliary adenocarcinoma, heptacellular carcinoma, bladder carcinoma including transitional cell carcinoma, adenocarcinoma, and squamous carcinoma, renal cell adenocarcinoma, endometrial carcinomas including adenocarcinomas and mixed Mullerian tumors (carcinosarcomas), carcinomas of the endocervix, ectocervix, and vagina such as adenocarcinoma and squamous carcinoma, tumors of the skin like squamous cell carcinoma, basal cell carcinoma, melanoma, and skin appendage tumors, esophageal carcinoma, carcinomas of the nasopharynx and oropharynx including squamous carcinoma and adenocarcinomas, salivary gland carcinomas, brain and central nervous system tumors including tumors of glial, neuronal, and meningeal origin, tumors of peripheral nerve, soft tissue sarcomas and sarcomas of bone and cartilage. Also included are non-solid hematopoietic tumors, such as leukemias.

There are a wide variety of cell proliferative conditions for which the SPATIAL cell cycle inhibitors disclosed herein can provide therapeutic benefits, with the general strategy being the inhibition of an anomalous cell proliferation. It is contemplated that the subject cell cycle inhibitors can be useful for controlling any condition in which normal cell cycle regulation is dysfunctional. To illustrate, cell types which exhibit pathological or abnormal growth include various neoplasias, fibroproliferative disorders (such as involving connective tissues, as well as other disorders characterized by fibrosis, including for example, rheumatoid arthritis, insulin dependent diabetes mellitus, glomerulonephritis, cirrhosis, and scleroderma), smooth muscle proliferative disorders (such as atherosclerosis and restinosis), and chronic inflammation.

In addition to proliferative disorders, the treatment of differentiative disorders which result from de-differentiation of tissue accompanied by reentry into mitosis is contemplated herein. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Construction of expression vectors, cellular and viral transgene carriers, and the characterization of target cells for neuronal gene therapy have been described and can be readily adapted for delivery of nucleic acids encoding SPATIAL cell cycle inhibitors (see, for example, Suhr et al., Arch. Neurol., 50:1252-1268, 1993; Jiao et al., Nature, 362:450-453, 1993; Friedmann, Ann. Med., 24:411-417, 1992; and Freese et al., Nuc. Acid Res., 19:7219-7223, 1991). Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, for instance, Wilm's tumors.

In specific embodiments, therapeutic application of a SPATIAL cell cycle inhibitor, for example, by gene therapy using a nucleic acid encoding a SPATIAL cell cycle inhibitor, can be used in the treatment of a neuroglioma. Gliomas account for 40-50% of intracranial tumors at all ages of life. Despite the increasing use of radiotherapy, chemotherapy, and sometimes immunotherapy after surgery for malignant glioma, the mortality and morbidity rates have not substantially improved. Exogenous expression of, for example, a SPATIAL cell cycle inhibitor in the cell can be used to inhibit cell proliferation. It has been demonstrated that gene therapy can be used to target glioma cells for expression of recombinant proteins (e.g., Chen et al., Proc. Natl. Acad. Sci., 91:3054-3057, 1994). Thus, a gene construct for expressing the subject cell cycle inhibitors can be delivered to the tumor, preferably by sterotactic-dependent means. In preferred embodiments, the gene delivery system is a retroviral vector. Since rapidly growing normal cells are rare in the adult CNS, glioma cells can be specifically transduced with a recombinant retrovirus. For example, the retroviral particle can be delivered into the tumor cavity through an Ommaya tube after surgery, or alternatively, packaging fibroblasts encapsulated in retrievable immunoisolatory vehicles can be introduced into the tumor cavity. In order to increase the effectiveness and decrease the side effects of the retrovirus-mediated gene therapy, glioma-specific promoters can be used to regulate expression of the therapeutic gene. For example, the promoter regions of glial fibrillary acidic protein (GFAP) and myelin basis protein (MBP) can operably linked to a nucleic acid encoding a SPATIAL cell cycle inhibitor in order to direct glial cell-specific expression of the corresponding protein.

In another embodiment, a SPATIAL cell cycle inhibitor, for example, introduced by gene therapy, can be used to treat certain breast cancers. In preferred embodiments, expression of a nucleic acid encoding a SPATIAL cell cycle inhibitor is controlled at least in part by a mammary-specific promoter, a number of which are available (for review, see Gunzberg et al., Biochem. J., 283:625-632, 1992).

In similar fashion, gene therapy protocols involving delivery of nucleic acids encoding SPATIAL cell cycle inhibitors can be used in the treatment of malignant melanoma. In preferred embodiments, gene therapy protocols for treatment of melanomas include, in addition to the delivery of a nucleic acid encoding a SPATIAL cell cycle inhibitor, the delivery of a pharmaceutical preparation of the inhibitor by direct injection. For instance, U.S. Pal. No. 5,318,514 describes an applicator for the electroporation of nucleic acids into epidermal cells and can be used in accordance with the present disclosure. In other examples, microparticle bombardment, using for example a gene gun (Biolistic; Dupont), may be useful for introducing nucleic acids into cells on the body's surface.

In yet another embodiment, one or more subject nucleic acids encoding SPATIAL cell cycle inhibitors are delivered to a sarcoma, for instance, an osteosarcoma or Kaposi's sarcoma. In a representative embodiment, the nucleic acid is provided in a viral vector and delivered by way of a viral particle which has been derivatized with antibodies immunoselective for an osteosarcoma cell (see, for example, U.S. Pal. Nos. 4,564,517 and 4,444,744; and Singh et al., Cancer Res., 36:4130-4136, 1976).

In some embodiments the disclosed SPATIAL cell cycle inhibitors may be used to treat various epithelial cell proliferative disorders, for example, psoriasis; keratosis; acne; comedogenic lesions; verrucous lesions such as verruca plana, plantar warts, verruca acuminata, and other verruciform lesions marked by proliferation of epithelial cells; folliculitis and pseudofolliculitis; keratoacanthoma; callosities; Darier's disease; ichthyosis; lichen planus; molluscous contagiosum; melasma; Fordyce disease; and keloids or hypertrophic scars.

The subject method can also be used in the treatment of folliculitis, such as folliculitis decalvans, folliculitis ulerythematosa reticulata or keloid folliculitis. For example, a cosmetic preparation of a SPATIAL cell cycle inhibitor protein can be applied topically in the treatment of pseudofolliculitis, a chronic disorder occurring most often in the submandibular region of the neck and associated with shaving, the characteristic lesions of which are erythematous papules and pustules containing buried hairs.

In another aspect of the invention, the subject method can be used in conjunction with various periodontal procedures in which inhibition of epithelial cell proliferation in and around periodontal tissue is desired. Periodontal disease is characterized in part by increased mitotic activity in the basal epithelial layer of the sulcus, wherein dissolution of the connective tissue results in the formation of an open lesion. The application of SPATIAL cell inhibitory preparations to the periodontium can be used to inhibit proliferation of epithelial tissue and thus prevent periodontoclastic development.

### VII. Screening for Agents that Affect SPATIAL Activity

Described herein are methods for identifying agents with SPATIAL inhibitory activity. Also described are methods of identifying agents that interfere with an interaction between SPATIAL polypeptide and Uba3 polypeptide. Further contemplated is identification of agents that mimic SPATIAL growth inhibitor activity.

The compounds which may be screened in accordance with this disclosure include, but are not limited to peptides, antibodies and fragments thereof, and other organic compounds (for example, peptidomimetics, small molecules) that inhibit SPATIAL activity as described herein or interfere with an interaction between SPATIAL and Uba3. Such compounds may include, but are not limited to, peptides such as, for example, soluble peptides, including but not limited to members of random peptide libraries; (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

Other compounds which can be screened in accordance with this disclosure include but are not limited to small organic molecules that are able to gain entry into an appropriate cell and affect the expression of SPATIAL gene or some other gene involved in a SPATIAL-mediated pathway (for example, by interacting with the regulatory region or transcription factors involved in SPATIAL gene expression); or such compounds that affect an activity of a SPATIAL isoform or the activity of some other intracellular factor involved in a SPATIAL-mediated pathway, such as Uba3.

Computer modeling and searching technologies permit identification of compounds, or the improvement of already identified compounds that can modulate expression or activity of a SPATIAL isoform. Examples of molecular modeling systems are the CHARMM and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen et al. Acta Pharmaceutical Fennica 97:159-166, 1988; Ripka, New Scientist 54-57, 1988; McKinaly and Rossmann, Annu Rev Pharmacol Toxicol 29:111-122, 1989; Perry and Davies, OSAR: Quantitative Structure-Activity Relationships in Drug Design pp. 189-193, 1989 (Alan R. Liss, Inc.); Lewis and Dean, Proc R Soc Lond 236:125-140 and 141-162, 1989; and, with respect to a model receptor for nucleic acid components, Askew et al., J Am Chem Soc 111:1082-1090, 1989. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

### 1. Screening for SPATIAL Inhibitory Agents

Disclosed herein are methods of identifying agents with potential for improving immune function, for example by increasing thymocyte numbers, by determining SPATIAL inhibitory activity of the agents. Any agent capable of inhibiting any biological activity of SPATIAL is contemplated. In some embodiments, a SPATIAL inhibitory agent interferes with an interaction between SPATIAL and Uba3, which is discussed below. In other embodiments, a SPATIAL inhibitory agent counteracts SPATIAL-induced growth suppression in vitro.

Screening assays may be conducted in a variety of ways. For example, one method would involve transiently transfecting cells with a SPATIAL expression vector and separating SPATIAL-expressing cells for use in cell-growth assays. Any eukaryotic cells or cell line may be used for transfections, such as 293T, NIH373, Wehi 7.2, 293F, or Cos7 cell lines. In one embodiment, cells may be transfected with an EGFP-SPATIAL expression vector as described, for instance, in Example 8, in which case SPATIAL transfectants could be identified by EGFP fluorescence and, optionally, could be separated or analyzed by fluorescence activated cell sorting (FACS; also called flow cytometry). Test compounds would be applied to SPATIAL-transfected cells and cell growth evaluated over time, for example at 24, 48 and 72 hours following addition of the test compound. SPATIAL inhibitory compounds would be identified by an increase in cell number as compared to control.

In another method, cells can be co-transfected with SPATIAL expression vector and a vector expressing a nucleic acid encoding a test protein or peptide. Cell growth assays would be performed as described above, and a SPATIAL inhibitory protein or peptide identified by its ability to overcome SPATIAL-induced growth suppression of transfected cells.

2. Screening for Compounds that Interfere with SPATIAL/Uba3 Interaction

In vitro systems may be designed to identify compounds capable of interfering with an interaction between SPATIAL and Uba3. Compounds identified may be useful, for example, in modulating an activity of SPATIAL isoforms, and thereby increasing thymocyte number.

The principle of assays used to identify compounds that interfere with an interaction between SPATIAL and Uba3 involves preparing a reaction mixture of a SPATIAL polypeptide, fragment or functional variant and an Uba3 polypeptide, fragment or functional variant under conditions and for a time sufficient to allow the two components to interact and form a complex. Thereafter, a test compound is added to the reaction mixture and various means are used to determine if the SPATIAL/Uba3 complex is affected by the test compound.

The screening assays can be conducted in a variety of ways. For example, one method to conduct such an assay would involve anchoring a SPATIAL polypeptide, peptide or fusion protein onto a solid surface, adding an Uba3 polypeptide, peptide or fusion protein to the reaction vessel, and adding the test substance and detecting SPATIAL/Uba3 complexes anchored on the solid phase at the end of the reaction. In one embodiment of such a method, SPATIAL may be anchored onto a solid surface, and Uba3, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component and test compound are added to the coated surface containing the anchored component. After the reaction is complete, unreacted components are removed (for example, by washing) under conditions such that any SPATIAL/Uba3 complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; for example, using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; for example, using an immobilized antibody specific for a SPATIAL protein, polypeptide, peptide or fusion protein or an Uba3 protein, polypeptide, peptide or fusion protein to anchor any complexes formed in solution, and a labeled antibody specific for the other component of the possible complex to detect anchored complexes.

### 3. Screening for Agents Having SPATIAL-Like Activity

Other methods contemplated herein include identifying agents that mimic or enhance SPATIAL activity, for example to interfere with cellular proliferation, such as to inhibit hyperproliferative disorders, such as neoplasia.

Agents that mimic or enhance SPATIAL activity can include, for example, agents that induce or increase SPATIAL expression in one or more cells; or agents that interact with SPATIAL and enhance its activity; or SPATIAL peptides having a desired SPATIAL activity; or molecules designed to have a SPATIAL structure that mediates a particular SPATIAL activity.

In some embodiments, agents that that induce or increase SPATIAL expression in one or more cells may be identified by contacting a biological system (such as a cell or FTOC) that expresses or is capable of expressing SPATIAL with an agent. SPATIAL expression or activity in the biological system may be measured in response to contact with the agent by methods well known in the art and described elsewhere in this disclosure. For instance, trans-acting coactivators of the SPATIAL gene regulatory region may be expected to increase SPATIAL activity. In other embodiments, agents may increase the half-life of the SPATIAL protein or its mRNA and thereby increase SPATIAL activity.

In other embodiments, agents that interact with SPATIAL and enhance its activity are contemplated. These agents may be identified, for example, by first identifying agents that interact with SPATIAL. Biophysical methods of accomplishing this step are well known in the art and include, for example, co-immunoprecipitation, yeast two-hybrid system, and GST pulldown assay, cross-linking of small molecules to SPATIAL, among other methods. Specific details of co-immunoprecipitation, yeast two-hybrid system, and GST pulldown assay are described in the Examples of this disclosure. Agents that interact with SPATIAL are then screened for enhancement of SPATIAL activity. For example, an agent is introduced into a biological system that expresses SPATIAL (such as, thymic stromal cells in culture, a FTOC, or cells that have been transfected with SPATIAL), and enhancement of a SPATIAL activity is measured. For example, introduction of the agent results in more rapid or profound inhibition of the cell cycle in the biological system.

In some embodiments, SPATIAL activity may be increased by agents that enhance an interaction between SPATIAL and Uba3, or between SPATIAL and other SPATIAL binding partners.

In other embodiments, SPATIAL peptides having a desired SPATIAL activity, such the ability to affect cell proliferation, may be identified by mapping experiments such as those described in Example 8. Representative SPATIAL peptides having SPATIAL growth regulatory activity include, for example, amino acids 21-197, 91-146 or 145-197 of SPATIAL(S) (SEQ ID NO: 2), or amino acids 21-231, 91-176 or 91-231 of SPATIAL(L) (SEQ ID NO: 4).

In still other embodiments, molecules can be designed to have a SPATIAL structure that mediates a particular SPATIAL activity using modeling analyses previously described. Candidate agents designed, for example in silico, to assume a SPATIAL structure may then be screen for desired SPATIAL activity as previously discussed.

### VI. Administration of Therapeutic Agents

This disclosure contemplates therapeutic agents useful for affecting cell proliferation. In some examples, the agents improve immune function and, in particular examples, increase thymocyte number. These agents include, without limitation, SPATIAL inhibitory agents and agents that interfere with an interaction between SPATIAL and Uba3. In other examples, therapeutics useful for inhibiting progression of the cell cycle are disclosed. These agents include, for example, SPATIAL polypeptides, peptides, nucleic acids and derivatives thereof (such as, SPATIAL variants and mimetics). Delivery systems and treatment regimens useful for such agents are known and can be used to administer these agents as therapeutics. In addition, representative embodiments are described below.

### 1. Administration of Nucleic Acid Molecules

In some embodiments where the therapeutic molecule is itself a nucleic acid (for example, siRNA, ribozyme or anti-sense oligonucleotide) or where a nucleic acid encoding a therapeutic protein or peptide is contemplated, administration of the nucleic acid may be achieved by an appropriate nucleic acid expression vector which is administered so that it becomes intracellular, for example, by use of a retroviral vector (see U.S. Pal. No. 4,980,286), or by direct injection, or by use of microparticle bombardment (for example, a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci., 88:1864-8, 1991). Alternatively, the nucleic acid can be introduced intracellularly and incorporated within host cell DNA for expression, for example, by homologous or non-homologous recombination.

The vector pcDNA is an example of a method of introducing the foreign cDNA into a cell under the control of a strong viral promoter (CMV) to drive the expression. However, other vectors can be used. Other retroviral vectors (such as pRETRO-ON, Clontech) also use this promoter but have the advantages of entering cells without any transfection aid, integrating into the genome of target cells only when the target cell is dividing. It is also possible to turn on the expression of a therapeutic nucleic acid by administering tetracycline when these plasmids are used. Hence these plasmids can be allowed to transfect the cells, then administer a course of tetracycline to achieve regulated expression.

Other plasmid vectors, such as pMAM-neo (also from Clontech) or pMSG (Pharmacia) use the MMTV-LTR promoter (which can be regulated with steroids) or the SV10 late promoter (PSVL, Pharmacia) or metallothionein-responsive promoter (PBPV, Pharmacia) and other viral vectors, including retroviruses. Examples of other viral vectors include adenovirus, AAV (adeno-associated virus), recombinant HSV, poxviruses (vaccinia) and recombinant lentivirus (such as HIV). All these vectors achieve the basic goal of delivering into the target cell the cDNA sequence and control elements needed for transcription. All forms of nucleic acid delivery are contemplated by this disclosure, including synthetic oligos, naked DNA, plasmid and viral, integrated into the genome or not.

Retroviruses have been considered a preferred vector for gene therapy, with a high efficiency of infection and stable integration and expression (Orkin et al., Prog. Med. Genet. 7:130-142, 1988). A nucleic acid therapeutic agent can be cloned into a retroviral vector and driven from either its endogenous promoter (where applicable) or from the retroviral LTR (long terminal repeat). Other viral transfection systems may also be utilized for this type of approach, including adenovirus, adeno-associated virus (AAV) (McLaughlin et al., J. Virol. 62:1963-1973, 1988), Vaccinia virus (Moss et al., Annu. Rev. Immunol 5:305-324, 1987), Bovine Papilloma virus (Rasmussen et al., Methods Enzymol 139:642-654, 1987) or members of the herpesvirus group such as Epstein-Barr virus (Margolskee et al., Mol Cell Biol. 8:2837-2847, 1988).

Recent developments in gene therapy techniques include the use of RNA-DNA hybrid oligonucleotides, as described by Cole-Strauss et al. (Science 273:1386-1389, 1996). This technique may allow site-specific integration of cloned sequences, thereby permitting accurately targeted gene replacement.

In addition to delivery of a nucleic acid therapeutic sequence to cells using viral vectors, it is possible to use non-infectious methods of delivery. For instance, lipidic and liposome-mediated gene delivery has recently been used successfully for transfection with various genes (for reviews, see Templeton and Lasic, Mol. Biotechnol., 11:175 180, 1999; Lee and Huang, Crit. Rev. Ther. Drug Carrier Syst., 14:173-206, 1997; and Cooper, Semin. Oncol., 23:172-187, 1996). For instance, cationic liposomes have been analyzed for their ability to transfect monocytic leukemia cells, and shown to be a viable alternative to using viral vectors (de Lima et al., Mol. Membr. Biol., 16:103-109, 1999). Such cationic liposomes can also be targeted to specific cells through the inclusion of, for instance, monoclonal antibodies or other appropriate targeting ligands (Kao et al., Cancer Gene Ther., 3:250-256, 1996).

### 2. Administration of Polypeptides or Peptides

In some embodiments, therapeutic agents comprising polypeptides or peptides may be delivered by administering to the subject a nucleic acid encoding the polypeptide or peptide, in which case the methods discussed in the section entitled “Administration of Nucleic Acid Molecules” should be consulted. In other embodiments, polypeptide or peptide therapeutic agents may be isolated from various sources and administered directly to the subject. For example, a polypeptide or peptide may be isolated from a naturally occurring source. Alternatively, a nucleic acid encoding the polypeptide or peptide may be expressed in vitro, such as in an E. coli expression system, as is well known in the art, and isolated in amounts useful for therapeutic compositions.

### 3. Methods of Administration, Formulations and Dosage

Methods of administering a therapeutic agent disclosed herein include, but are not limited to, intrathymic, intrathecal, intradermal, intramuscular, intraperitoneal (ip), intravenous (iv), subcutaneous, intranasal, epidural, and oral routes. The therapeutics may be administered by any convenient route, including, for example, infusion or bolus injection, topical, absorption through epithelial or mucocutaneous linings (for example, oral mucosa, rectal and intestinal mucosa, and the like) ophthalmic, nasal, and transdermal, and may be administered together with other biologically active agents. Administration can be systemic or local. In addition, it may be desirable to introduce a pharmaceutical composition by any suitable route, including intraventricular and intrathecal injection; intraventricular injection may be facilitated by an intraventricular catheter, for example, attached to a reservoir. Pulmonary administration can also be employed (for example, by an inhaler or nebulizer), for instance using a formulation containing an aerosolizing agent.

In a specific embodiment, it may be desirable to administer a pharmaceutical composition locally to the area in need of treatment. This may be achieved by, for example, and not by way of limitation, local or regional infusion or perfusion during surgery, topical application (for example, wound dressing), injection, catheter, suppository, or implant (for example, implants formed from porous, non-porous, or gelatinous materials, including membranes, such as sialastic membranes or fibers), and the like. In one embodiment, administration can be by direct injection at the site (or former site) of a tissue that is to be treated, such as the thymus. In another embodiment, the therapeutic are delivered in a vesicle, in particular liposomes (see, e.g., Langer, Science 249, 1527, 1990; Treat et al., in Liposomes in the Therapy of Infectious Disease and Cancer, Lopez-Berestein and Fidler (eds.), Liss, N.Y., pp. 353-365, 1989).

Some agents of this disclosure whose only (or only substantial) biological activity is to inhibit SPATIAL activity may be administered systemically with diminished risk of global side effects because the expression of SPATIAL is predominantly limited to thymus and testes. As a result of SPATIAL's limited tissue-specific expression, SPATIAL inhibitory agents with high specificity would be expected to predominantly effect only the thymus and testes.

In yet another embodiment, the therapeutic can be delivered in a controlled release system. In one embodiment, a pump may be used (see, e.g., Langer Science 249, 1527, 1990; Sefton Crit. Rev. Biomed. Eng. 14, 201, 1987; Buchwald et al., Surgery 88, 507, 1980; Saudek et al., N. Engl. J. Med. 321, 574, 1989). In another embodiment, polymeric materials can be used (see, e.g., Ranger et al., Macromol Sci. Rev. Macromol Chem. 23, 61, 1983; Levy et al., Science 228, 190, 1985; During et al., Ann. Neurot. 25, 351, 1989; Howard et al., J. Neurosurg. 71, 105, 1989). Other controlled release systems, such as those discussed in the review by Langer (Science 249, 1527 1990), can also be used.

The vehicle in which the agent is delivered can include pharmaceutically acceptable compositions known to those with skill in the art. For instance, in some embodiments, therapeutic agents disclosed herein are contained in a pharmaceutically acceptable carrier. The term “pharmaceutically acceptable” means approved by a regulatory agency of the federal or a state government or listed in the U.S. Pharmacopoeia or other generally recognized pharmacopoeia for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions, blood plasma medium, aqueous dextrose, and glycerol solutions can also be employed as liquid carriers, particularly for injectable solutions. The medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic, if desired, can also contain minor amounts of wetting or emulsifying agents, or pH buffering agents. The therapeutic can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. A more complete explanation of parenteral pharmaceutical carriers can be found in Remington: The Science and Practice of Pharmacy (19th Edition, 1995) in chapter 95.

Embodiments of other pharmaceutical compositions are prepared with conventional pharmaceutically acceptable counterions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

Therapeutic agents of this disclosure can be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sacheffe indicating the quantity of active agent. Where the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to administration.

The amount of the therapeutic that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each patient's circumstances. An example of such a dosage range is 0.1 to 200 mg/kg body weight in single or divided doses. Another example of a dosage range is 1.0 to 100 mg/kg body weight in single or divided doses.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

The therapeutic agents of the present disclosure can be administered at about the same dose throughout a treatment period, in an escalating dose regimen, or in a loading-dose regime (for example, in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some embodiments long-term treatment with the drug is contemplated, for instance in order to reduce the occurrence of expression or overexpression of the target gene (such as, SPATIAL).

In some embodiments, sustained intra-thymic (or near-thymic) release of the pharmaceutical preparation that comprises a therapeutically effective amount of the particular therapeutic agent may be beneficial. Slow-release formulations are known to those of ordinary skill in the art.

In some embodiments, a therapeutic agent of the present disclosure is administered to a subject before, concurrent with and/or after a bone marrow transplant. Administration of the agent prior to bone marrow transplant is thought to condition the thymus for receipt of donor hematopoietic stem cells. For example, inhibition of SPATIAL may permit thymic stromal cells to proliferate and thereby create a thymic microenvironment that facilitates proliferation and/or differentiation of the donor hematopoietic stem cells when they migrate to the thymus following bone marrow transplant. It may be useful to administer (or first administer) the therapeutic agent at least 30 days, at least 14 days, at least 7 days, at least 5 days, at least 3 days, or at least 1 day prior to a bone marrow transplant. In other embodiments, the therapeutic agent is first administered at any time after a bone marrow transplant and prior to immune system reconstitution in the recipient; for example, the therapeutic agent may be administered (or first administered) 1 day, 7 days, 14 days, 30 days, 60 days or longer after bone marrow transplant. In other embodiments, the therapeutic agent is first administered prior to bone marrow transplant and administration is continued until the immune system is substantially reconstituted, for example, when T cell count is at least the lower limit of the normal range or the subject is immune competent in resisting disease and/or opportunistic infection.

The following examples are provided to illustrate certain particular features and/or embodiments. These examples should not be construed to limit the invention to the particular features or embodiments described.

### EXAMPLES

### Example 1

### Isolation and Characterization of SPATIAL Nucleic Acids

This example describes the cloning and characterization of the SPATIAL cDNA and genomic sequences.

A cDNA library was made from day 14 fetal thymic organ culture (FTOC) treated with 2-deoxyguanosine to remove thymocytes. Residual thymocytes were removed with magnetic beads coupled to anti-CD45 antibodies to produce a highly purified preparation of thymic stromal cells. The cDNA library was further enriched for thymic stromal cell-specific gene expression by sequential subtraction hybridization against NIH3T3 and spleen mRNA (Kim, et al., J. Immunol. Methods, 213(2):169-182, 1998).

The resulting cDNA clones were then screened for tissue expression in thymus and other organs. One of these genes, named SPATIAL (Stromal Protein Associated with Thymii And Lymph node), is alternatively spliced to generate two mRNAs in mouse thymus.

The original cDNA clone was incomplete. The missing 5′sequence was obtained by repeated cDNA library screenings and multiple rounds of RACE (rapid amplification of cDNA ends). Two cDNAs that differ by 102 base pairs were cloned (SEQ ID NOs: 1 and 3). Reverse transcriptase polymerase chain reaction (RT-PCR) assays were used to confirm that the cDNAs were expressed as mRNAs in thymus. Primer extension and S1 nuclease assays were also used to map the 5′ end of the SPATIAL message and confirmed that full-length cDNAs had been cloned.

Genomic DNA encompassing the entire mouse SPATIAL coding region and 3 kb of the SPATIAL gene promoter was cloned and sequenced. The SPATIAL gene covers about 12,000 bases and is divided into 4 exons. The majority of the protein is encoded by exon 1. Exon 3 contains an alternative splice site that generates the short form of SPATIAL (SEQ ID NO: 1). The promoter is unusual in that no consensus TATA box or Kozak site was found. TATA-less promoters have been described before and are often associated with genes that are not regulated transcriptionally (Azizkhan et al., Crit. Rev. Eukarol. Gene. Expr., 3(4):229-254, 1993).

BLAST and FASTA homology searches using SPATIAL cDNA and protein sequences did not reveal any significant matches to the sequence databases of known genes and proteins, respectively. Analysis of the SPATIAL protein using PROPSEARCH (Hobohm and Sander, J. Mol. Biol., 251(3):390-399, 1995) indicated that SPATIAL has an 80 to 87% probability of being related to a number of homeobox, POU domain, and leucine zipper transcription factors. ClustalW analysis of SPATIAL and the genes identified by PROPSEARCH suggested a weak homology.

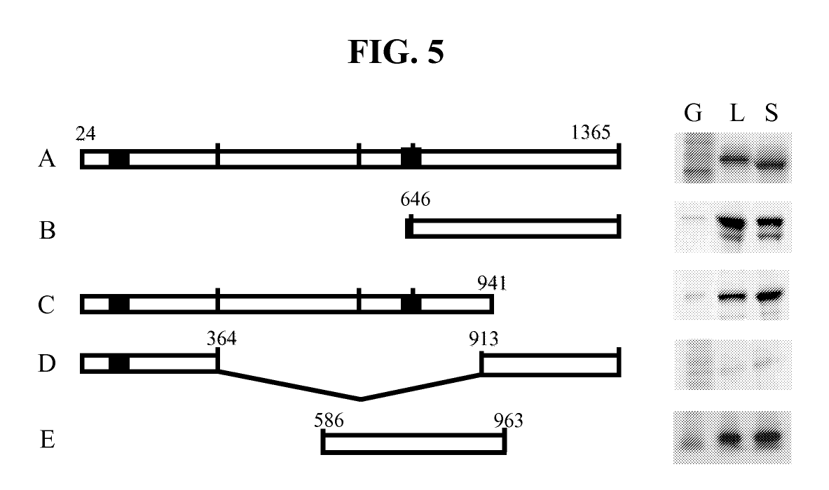

SPATIAL mRNAs expressed in the thymus were predicted by Psort software (Nakai et al., Genomics, 14:897-911, 1992) to encode proline-rich proteins with putative nuclear localization motifs. Both isoforms of SPATIAL fused to the enhanced green fluorescent protein (EGFP) localized to the nucleus in transfected cells while cells transfected with EGFP alone (PEGFPN1; Clontech) did not show appreciable nuclear localization (see, FIG. 5 of Flomerfelt et al., Genes and Immunity, 1:391-401, 2000).

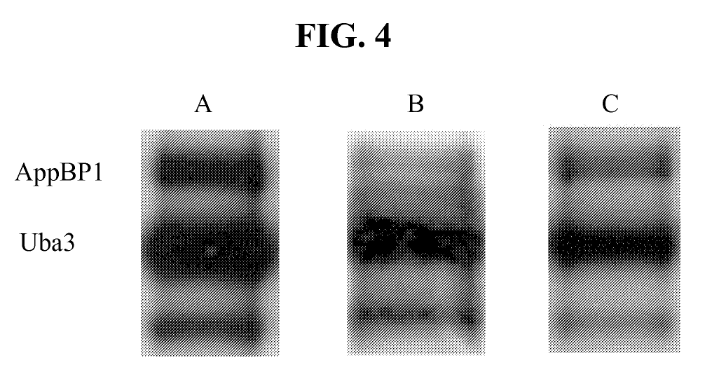

A Southern blot genomic DNA analysis using SPATIAL cDNA as a probe revealed that the SPATIAL gene is conserved among mouse, rat, human, monkey, rabbit, cow and dog (see, FIG. 4 of Flomerfelt et al., Genes and Immunity, 1:391-401, 2000). The human homolog of mouse SPATIAL (GenBank accession XM_{—}166127) is found on chromosome 10 and it was shown to be expressed in fetal and adult thymus.

### Example 2

### Tissue, Developmental and Cell-Type SPATIAL Expression Patterns

This example describes the characterization of SPATIAL expression patterns, and indicates that SPATIAL expression is independent of the presence of thymocytes, but dependent on stromal cell organization, and is localized to the thymic subcapsule.

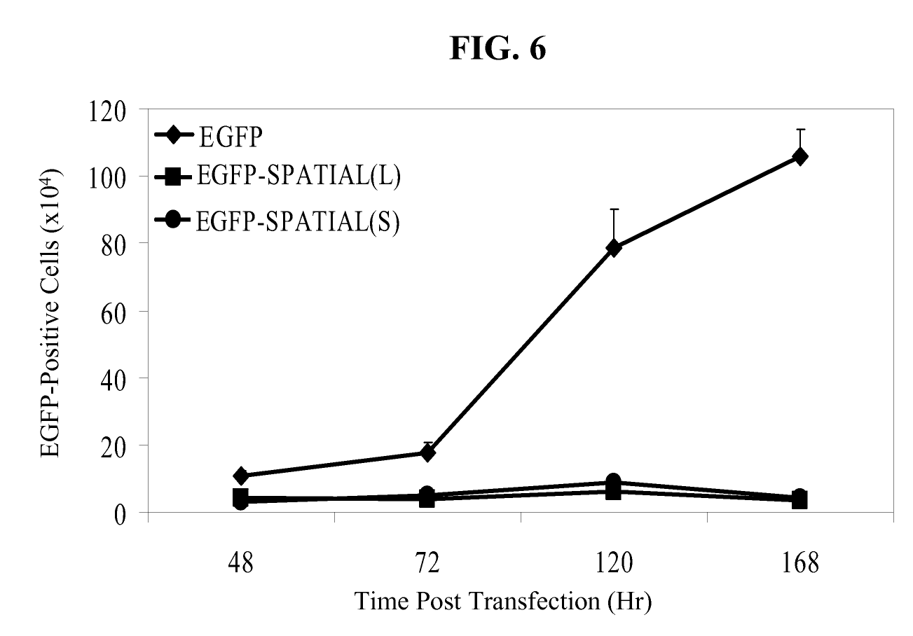

A RT-PCR assay was developed to detect both SPATIAL isoforms simultaneously, as described in Flomerfelt et al., Genes and Immunity, 1:391-401, 2000. In addition to expression in the thymus and lymph node, as described in Example 1, both SPATIAL isoforms were also expressed in the kidney and in skeletal muscles, but at much lower levels. SPATIAL was also expressed in fetal liver, but liver expression was not detected in three week-old mice. Multiple splicing variants of SPATIAL are expressed in the testis (Irla et al., Gene Exp. Patt., 3:135-138, 2003). SPATIAL expression was not detected using a sensitive RT-PCR assay in the spleen, heart, brain, liver, large intestine, small intestine, bone marrow, or peyer patches of 4-6 week old mice (see, for example, FIG. 6a in Flomerfelt et al., Genes and Immunity, 1:391-401, 2000).

SPATIAL is developmentally regulated in the mouse fetus. An expression analysis in whole fetus, staged by crown to rump measurement, was done and showed that the gene was not detectable at day 9 of fetal life. The short form of SPATIAL begins its expression at day 10. This pattern persisted until day 12-13 when both SPATIAL isoforms were observed (see, for example, FIG. 6b in Flomerfelt et al., Genes and Immunity, 1:391-401, 2000). The initiation of SPATIAL expression in the fetus coincides with the formation of the earliest thymic rudiment and precedes its colonization by thymocytes at day 11 (Suniara et al., Eur. J. Immunol., 29(1):75-80, 1999; Amagai et al., Eur. J. Immunol., 25(3): 757-762, 1995). Thymic expression of both isoforms of SPATIAL is detected in mouse embryos at day 12 and continues throughout life.

SPATIAL expression cannot be detected in thymocytes using RT-PCR on RNA obtained from highly purified CD45+ thymocytes prepared by flow cytometer sorting from normal mice. Actin and cyclophilin mRNA was readily detected using the same RNA samples. To examine SPATIAL expression in stromal cells, thymii from day 14 mouse fetuses were harvested and cultured in fetal thymic organ culture (FTOC). The FTOC was specifically depleted of thymocytes using 2-deoxyguanosine treatment. 2-deoxyguanosine is a nucleotide analog that is toxic to thymocytes. After 14 days, flow cytometric analysis showed that greater than 98% of lymphoid cells had been depleted leaving a highly enriched stromal cell preparation. These cultures can be re-colonized by stem cells and are fully capable of supporting thymopoiesis (Hare et al., Semin. Immunol., 11(1):3-12, 1999). Using RT-PCR, it was shown that SPATIAL is abundantly expressed in thymic stromal cells obtained from FTOC (Flomerfelt et al., Genes and Immunity, 1:391-401, 2000).

Semi quantitative PCR analysis suggested that the amount of SPATIAL mRNA in the thymocyte-depleted FTOC was roughly equivalent to that expressed in intact day 14 thymus. FTOC treated with 2-deoxyguanosine can be trypsinized to obtain a suspension of thymic stromal cells. This stromal cell suspension can be pelleted by centrifugation, alone or with added thymocytes, and transferred to organ culture. In such a reaggregate thymic organ culture (RTOC), the cell pellet rapidly reforms into an intact 3-dimensional thymus lobe that can support thymopoiesis. SPATIAL expression was maintained in RTOC in the presence or absence of added thymocytes. However, SPATIAL expression was rapidly and irreversibly lost if the thymic stromal cell suspension was grown on plastic in a two-dimensional culture system.

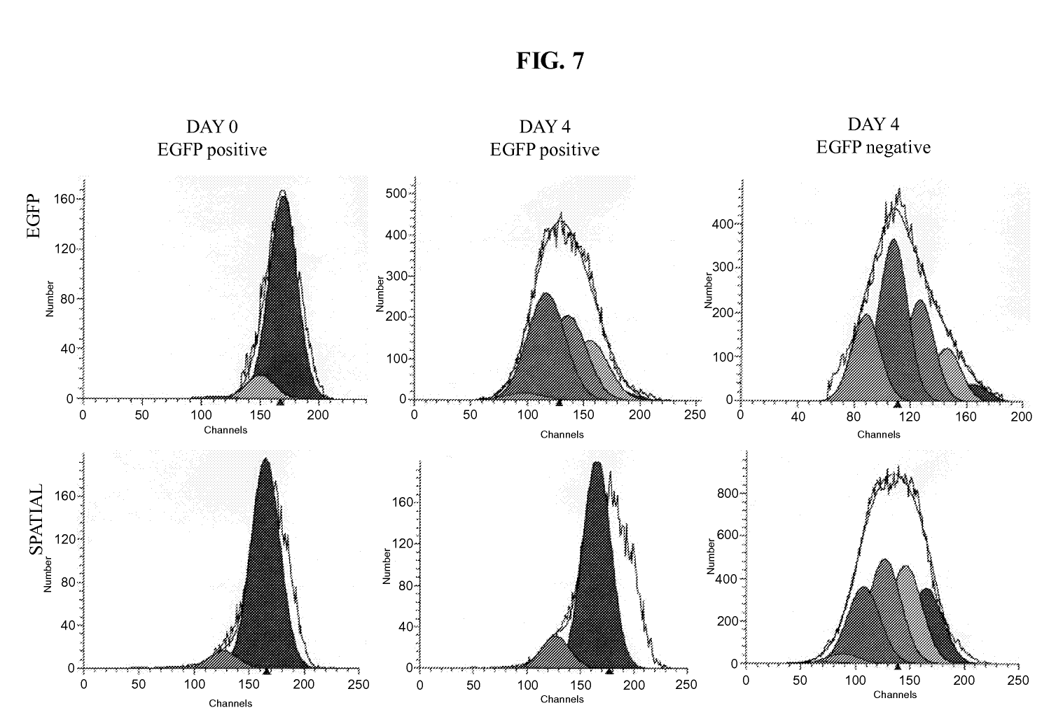

SPATIAL expression in several mutant mice blocked at different stages of thymocyte development was examined. Thymic stromal cell composition and organization are disrupted in recombination activating gene 2 (Rag2), TCRα, TCRδ, and CD3ε knock out mice (Klug et al., Proc. Natl. Acad. Sci., 95(20):11822-11827, 1998; Naquet et al., Semin. Immunol., 11 (1):47-55, 1999). Despite this, both isoforms of the SPATIAL gene were expressed in the thymii of these animals. SPATIAL was also highly expressed in the highly disorganized thymic rudiment of the dominant-negative Ikaros mutant mouse that contains virtually no lymphocytes (see, for example, FIG. 7a in Flomerfelt et al., Genes and Immunity, 1:391-401, 2000).

An affinity-purified rabbit antiserum against a C-terminal SPATIAL peptide was produced (as described in Flomerfelt et al., Genes and Immunity, 1:391-401, 2000). The antiserum immunoprecipitated both isoforms of H³-labelled, in vitro translated SPATIAL protein. The immunoprecipitation was blocked by the addition of 0.774 μM of the immunizing peptide. Human kidney epithelial cell line, 293T, was transfected with expression vectors containing HA-tagged SPATIAL. Protein lysates from transfected 293T cells were immunoprecipitated with anti-HA antibody and analyzed by Western blot using the SPATIAL antiserum. The anti-SPATIAL only reacted with proteins immunoprecipitated by anti-HA antibody from HA-SPATIAL transfected cells. The apparent mobility of the SPATIAL isoforms (38 and 32 kDa) in both experiments was greater than the predicted mass based on the cDNA sequence (long form 25.7 kDa and short form 23.3 CKD). The anti-SPATIAL antibody also detects 38 and 32 kDa proteins in Western blot analysis of thymus protein extracts.

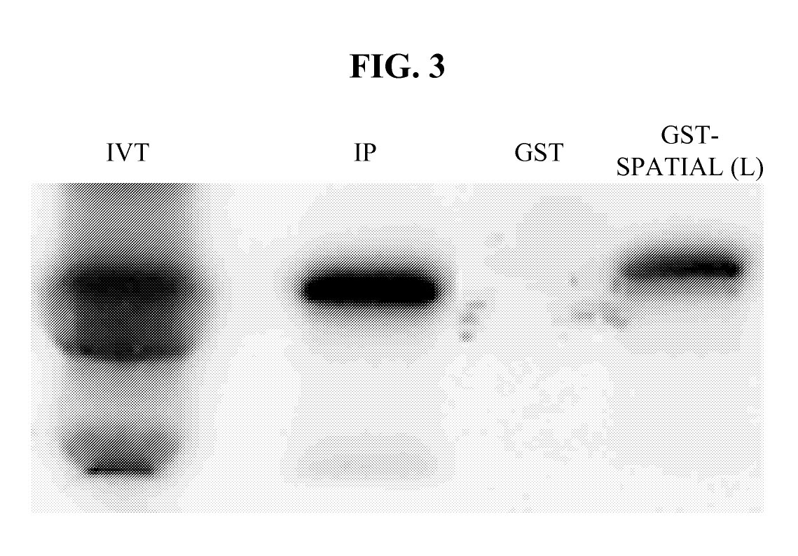

Immunohistochemistry on frozen thymus sections using SPATIAL anti-serum indicates that SPATIAL expression is concentrated within the thymic subcapsule (see, for example, FIG. 3 in Flomerfelt et al., Genes and Immunity, 1:391-401, 2000). The staining was blocked by the addition of the immunizing peptide but not with an irrelevant peptide. No staining was noted when the anti-SPATIAL antibody was omitted during the staining procedure.

Taken together, this example indicates that SPATIAL expression (i) is independent of the presence of thymocytes since it precedes the migration of thymocytes into the fetal thymus, occurs in mutant mice that lack appreciable number of thymocytes, and is maintained in thymocyte depleted FTOC; (ii) is dependent on stromal cell organization; and (ii) is localized to the thymic subcapsule, which is a region that is associated with thymocyte precursor cells.

### Example 3

### Generation of SPATIAL Knock Out Mice

The entire known coding region of the SPATIAL gene was deleted in mice by homologous recombination. The knock out vector was constructed using a P1 clone that contained the SPATIAL gene derived from 129 mice (e.g., Murray, Lambda II, eds. Hendrix et al., New York: Cold Spring Harbor Laboratory, 1983, pp. 395-432). The P1 clone was identified by PCR using two primer sets specific for the 5′ and 3′ ends of the SPATIAL cDNA. The P1 clone was digested with PST1 or Bam H1 and the resulting fragments were cloned into pSK⁺. Colony hybridization using a full-length cDNA probe was used to identify genomic clones that contained coding regions of SPATIAL. The resulting clones were sequenced and assembled.

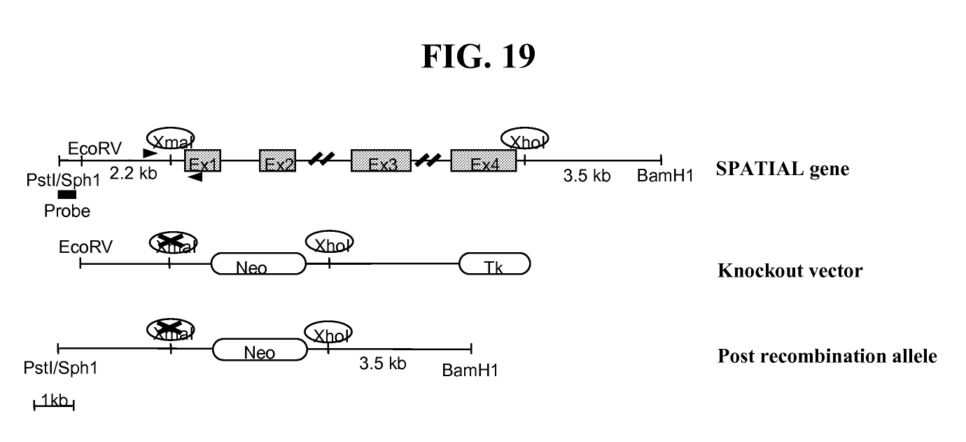

The SPATIAL gene is composed of 4 exons found on mouse chromosome 10 (see FIG. 19). The first exon is 142 bases long and contains the 5′ end of the predicted cDNA. The second and third exons are 247 and 260 bases long. The third exon contains an alternative splice site 102 bases from the 5′ end that results in the production of the short isoform of SPATIAL. The fourth exon is 365 bases and contains the terminal bases up to the stop codon of both SPATIAL isoforms.

The entire coding region of the gene is contained between a XmaI and an Xho site as shown in FIG. 19. This region was replaced with a floxed Neo gene to completely delete the coding portion of the SPATIAL gene. A thymidine kinase gene was cloned about 3 kb downstream of the stop codon to facilitate identification of homologous recombinants.

The knock out vector was linearized with ClaI and the targeting construct was purified and then electroporated into ES stem cells. One hundred thirty three Neo-resistant, TK: cell lines were obtained. Genomic DNA from these cells was purified and used for PCR analysis to screen for homologous recombination using one primer within the Neo gene of the targeting vector and another that was complementary to genomic DNA outside of the expected recombination site. One clone was identified using this procedure and homologous recombination was verified using Southern hybridization.

The identified clone was injected into blastocysts and five chimeric founder mice were obtained. These mice were bred to C57B1/6 mice and the presence of the deleted allele was detected using genomic DNA PCR. SPATIAL heterozygotes were interbred to obtain SPATIAL null mice that did not express detectable SPATIAL message by RT-PCR analysis in the thymus. Deletion of SPATIAL was not lethal and mice were born with expected Mendelian and sex ratios. SPATIAL null mice bred normally.

### Example 4

### Phenotypic Analysis of Aged SPATIAL Knock Out Mice

This example demonstrates that SPATIAL gene dosage affects thymocyte number in thymii of aged mice.

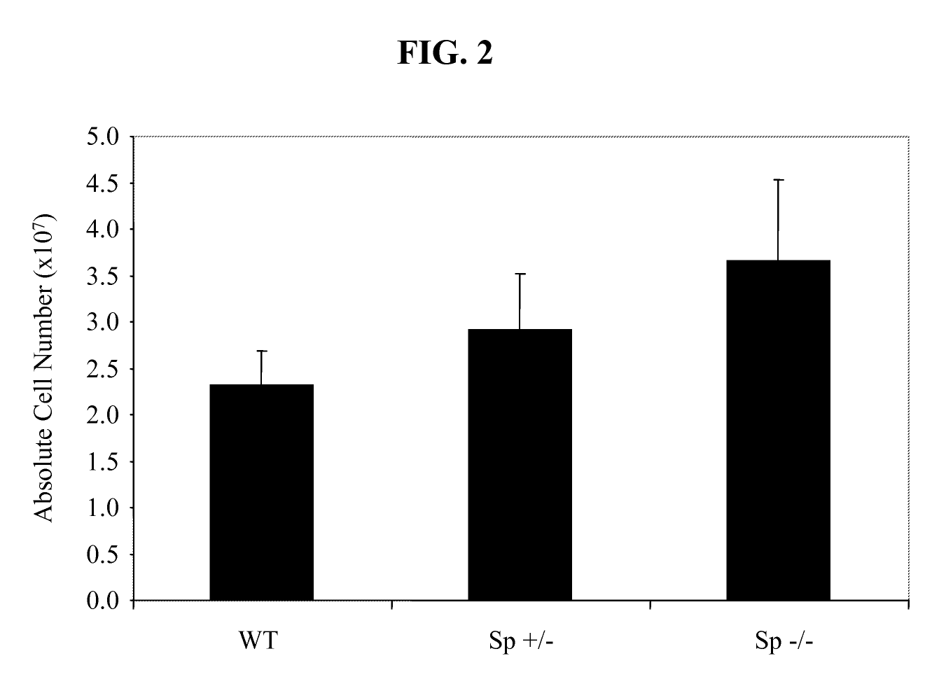

SPATIAL knock out mice appeared grossly normal in outward appearance. In 3-5 week old mice, there was little difference in the composition or size of the thymus. However, as the mice aged past 5 months it became obvious that the thymus of the SPATIAL knock out mice contained more thymocytes (identified by CD45 expression) than the wild type littermates. The difference in thymocyte numbers between the wild type and SPATIAL null mice became more dramatic as the mice aged. FIG. 2 shows total number of thymocytes in thymii of 10-12 month old wild type (n=2), SPATIAL heterozygote (Sp+/−; n=2) and SPATIAL double knock out (Sp −/−; n=6) littermates. Thymii of SPATIAL heterozygote and double knock mice each contain significant more thymocytes than wild type littermates. Moreover, the phenotype of the SPATIAL heterozygote indicates that even a partial decrease in SPATIAL activity may lead to increased thymocyte number in the thymus.

The knock out and wild type mice displayed an age-related decline in thymus size, but the number of thymocytes in the SPATIAL null mouse was consistently greater. The phenotype of the SPATIAL heterozygote was intermediate between the phenotypes of wild type and SPATIAL null littermates. These data demonstrate that thymus function is enhanced in both single and double SPATIAL knock out mice and that the thymus in these mice still responds to normal regulatory signals. In addition, the phenotype of the SPATIAL heterozygote indicates that the function of SPATIAL correlates with gene expression levels and that mechanisms to compensate for loss of SPATIAL expression are absent or of limited strength.

In aged SPATIAL null mice, the increase in thymus size was correlated with a greater percentage of T cells with a naive phenotype in the periphery compared to wild type littermates. Analysis of thymii from progressively older SPATIAL null and wild type mice showed that the first difference noted was an increase in the absolute numbers of DN cells. In aged mice (10-12 months), there was an average of 2-3 times more DN cells in SPATIAL null mice than in wild type littermates. However, the absolute number of thymocytes in all subsets was consistently greater in the SPATIAL null mouse.

### Example 5

### Analysis of SP/Rag2 DKO Mouse

This example demonstrates that SPATIAL activity directly affects the numbers of the DN cells in the thymus.

As shown in Example 4, DN cells were the earliest thymocyte population affected in the SPATIAL null. DN cells comprise about 1-3% of the total number of thymocytes. Because the numbers of DN cells are directly related to the numbers of DP and SP thymocytes (Almeida et al., J. Exp. Med., 194(5): 591-599, 2001), it hypothesized that an increase in DN cells contributed to the increased thymocyte number in the SPATIAL null thymus.

To facilitate analysis of thymic DN cells, the SPATIAL null mouse was crossed with the recombination activating gene 2 knock out (Rag2 null) mouse (Shinkai et al., Cell, 68:855-867, 1992) to create a double SPATIAL/Rag2 double knock out (SP/Rag2 DKO) mouse. The Rag2 mutation blocks thymocyte development at the DN stage due to a defect in T cell receptor rearrangement; thus, the SP/Rag2 DKO thymus contains only DN cells. This model permitted easier analysis of the DN population of cells since they were the majority of cells. In addition, the SP/Rag2 DKO mouse provided a useful immunodeficient model system to examine thymic reconstitution (as described in more detail in Example 7).

The thymii of the SP/Rag2 DKO mice contained about 10 times more thymocytes than the Rag2 null littermates. Although there were overall more DN cells in the SP/Rag2 DKO mice, the proportions of the DN subsets were normal. As expected in a Rag2 null background, no mature T cells were detected in any of the mice. Similar to the SPATIAL heterozygote discussed in Example 4, a SPATIAL heterozygote/Rag2 null mouse showed a phenotype intermediate between the Rag2 null and SP/Rag2 DKO mice, which further indicates a dosage effect for SPATIAL gene function.

Surprisingly, SP/Rag2 DKO mice showed a significant increase in thymus cell numbers at approximately 8-12 weeks of age as compared to Rag2 null littermates, which was considerably earlier than the similar phenotype observed in SPATIAL null mice as compared to wild type littermates, which is discussed in Example 4. This difference may be due to the fact that the SP/Rag2 DKO mice are completely lacking an adaptive immunity (that is, immunity acquired through responses of antigen-specific lymphocytes and resulting in immune memory) while the SPATIAL null mice are not.

### Example 6

### SPATIAL Knock Out does not Affect T Cell Selection

This example demonstrates that the SPATIAL gene knock out does not affect normal T cell development.

The SP/Rag2 DKO mice were crossed to the HY T cell receptor (HY TCR) transgenic mice (Shinkai et al., Cell, 68:855-867, 1992) to examine the effect of SPATIAL deficiency on thymocyte positive and negative selection. The HY TCR reacts with a male specific antigen derived from the SRY gene on the Y chromosome. In female HY TCR transgenic mice, efficient positive selection occurs and a large percentage of CD8⁺ SP cells are produced. In male mice, the HY TCR reacts with its cognate antigen and mediates negative selection resulting in a thymus devoid of DP and SP cells.

Analysis of the thymii of HY TCR transgenic SP/Rag2 DKO mice showed no evidence of defects in negative or positive selection. In female mice, there were abundant DP and CD8⁺ SP cells while in the male mice, very few DP cells and no CD8⁺ SP cells were detected. The same qualitative results were obtained with SPATIAL wild type RAG2 KO, HY TCR transgenic (control) littermates.

Consistent with earlier examples, however, an increase in thymocyte cell number was observed in the thymii of HY TCR transgenic SP/Rag2 DKO when compared to control littermates. This result demonstrates that inhibition of SPATIAL expression causes an increase in thymocyte number but does not affect normal T cell development.

### Example 7

### Bone Marrow Transplantation in SP/Rag2 Mice

This example demonstrates that inhibition of SPATIAL gene expression leads to rapid increases in thymocyte number and to rapid reconstitution of thymic function following bone marrow transplantation. Rag2 null and SP/Rag2 DKO mice were used as hosts for BMT to demonstrate the affect of SPATIAL on thymic reconstitution in immunodeficient animals.

Bone marrow was harvested from wild type Ly5.1 congenic mice and an anti-Ly5.1 antibody was used to identify transferred cells in the host mouse following BMT. Seven to ten million T cell-depleted bone marrow cells were injected into host mice aged 3-5 months. Age-matched littermates were used in each experiment.

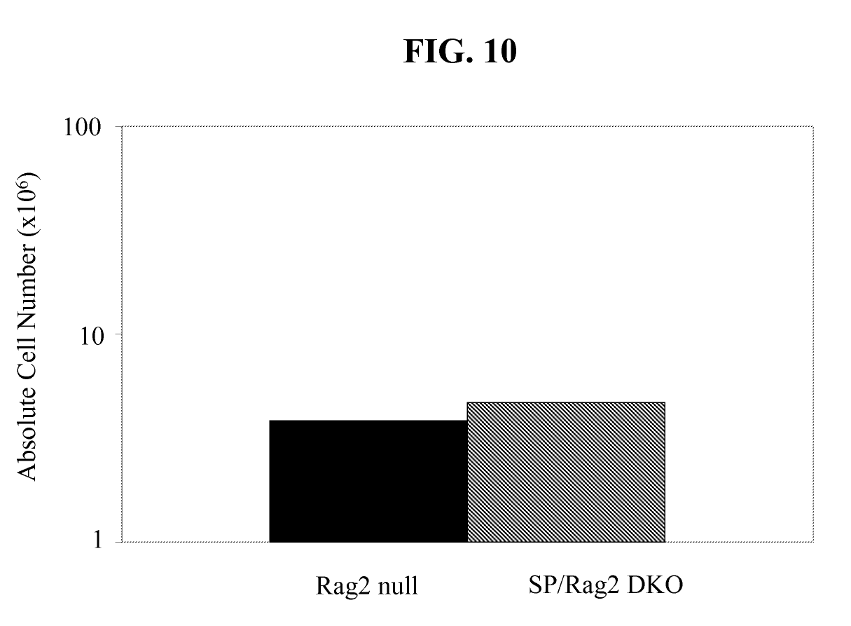

Ly5.1⁺plenic B cells were used to monitor the engraftment of the BMT. As shown in FIG. 10, there was no significant difference between the absolute numbers of Ly5.1⁺splenic B cells measured in Rag2 null and SP/Rag2 DKO mice at three weeks post-BMT. Thus, SPATIAL knock out does not affect the ability of the immune system to take up transferred cells after BMT.

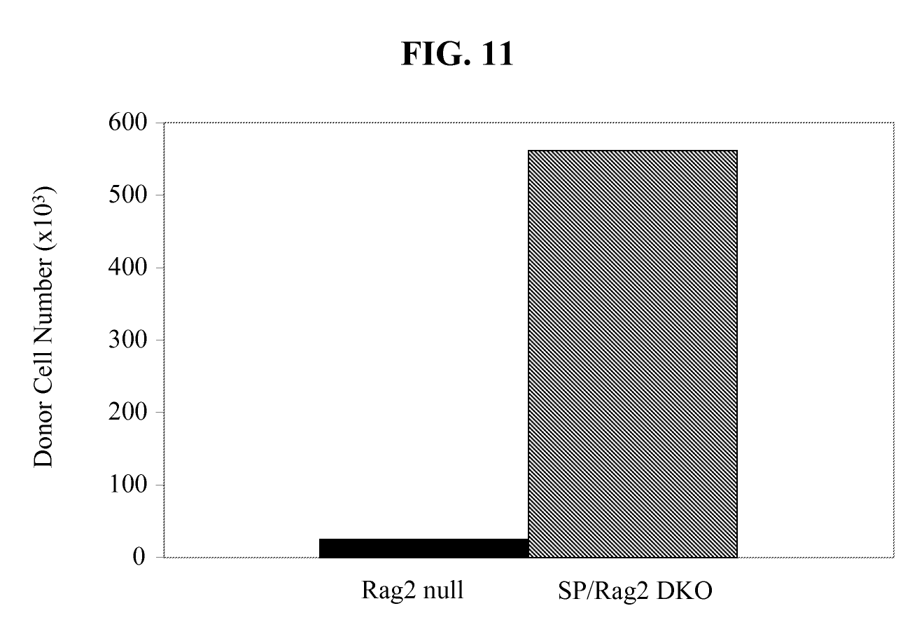

Surprisingly, thymic reconstitution in the SP/Rag2 DKO mice occurred faster and, as shown in FIG. 11, with a greater magnitude than in Rag2 null mice. Within 3 weeks of BMT, donor cells had entered the thymus of the SP/Rag2 DKO mice and all thymocyte developmental stages, except mature CD8⁺ single positive cells, were abundant. In Rag2 null mice, a comparable state of reconstitution was not reached for another 10-14 days.

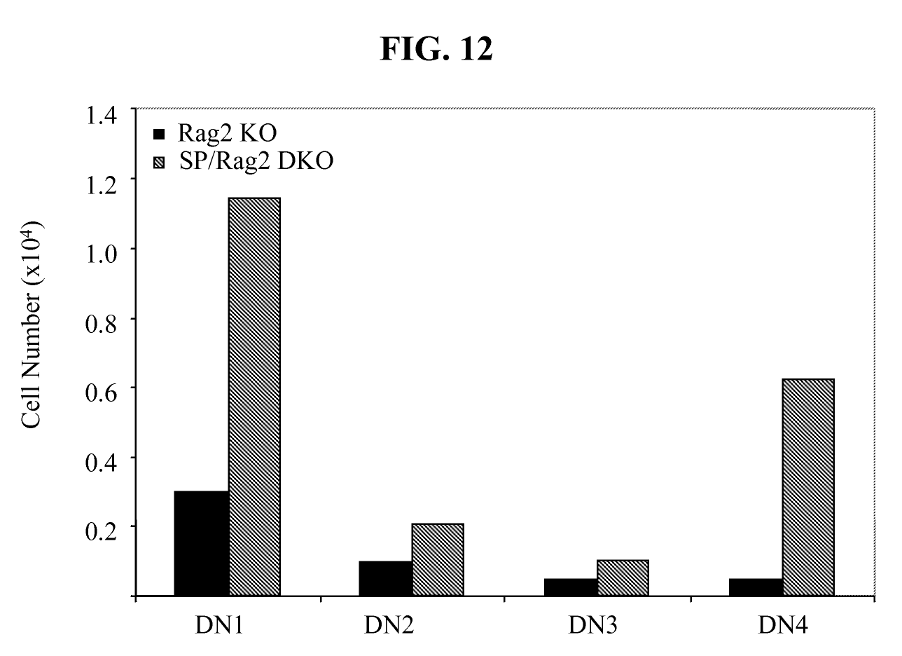

FIG. 12 shows that at three weeks post-BMT rapid thymic reconstitution is largely attributable to rapid accumulation of DN cells in the thymii of SP/Rag2 DKO mice as compared to Rag2 null mice. In particular, the numbers of DN1 and DN4 cells in SP/Rag2 DKO mice are disproportionately increased over the numbers of the corresponding cells in Rag2 null control mice. These data demonstrate that inhibition of SPATIAL gene expression leads to rapid increases in DN cells, particularly DN1 cells, in the thymus following BMT.

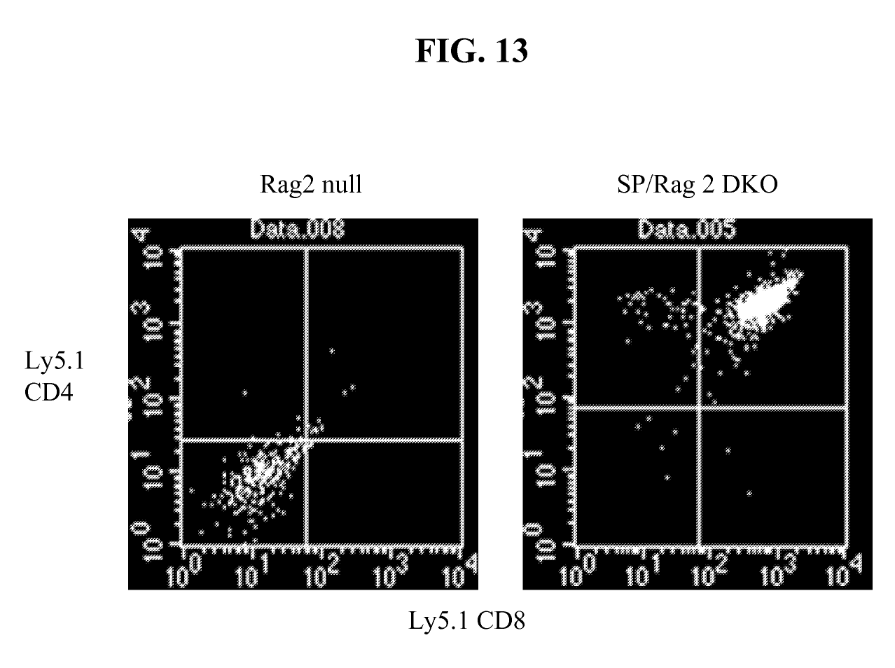

FIG. 13 shows a FACS analysis profile of thymocytes isolated at three week post-BMT from Rag2 null mice and SP/Rag2 DKO mice. Cells were labeled with fluorescent antibodies specific for CD4 and CD8 prior to FACS analysis. This figure demonstrates that no CD4⁺/CD8⁺ double-positive cells were present in the thymii of immunodeficient Rag2 null mice three weeks after receiving bone marrow transplantation; however, in the same time frame, numerous CD4⁺/CD8⁺ double-positive cells were present in thymii of previously immunodeficient SP/Rag2 DKO mice. Furthermore, at this time point, mature CD4⁺/CD8⁻ and CD4⁻/CD8⁺ single-positive cells were detected only in the SP/Rag2 DKO thymus. In addition, mature CD4-positive and CD8-positive cells were found in the periphery (lymph node and spleen) of SP/Rag2 DKO mice at 3 weeks post-BMT, while fewer such cells were found in the periphery of Rag2 null mice at that time point.

At 5 weeks post transplant, both the SP/Rag2 DKO and the Rag2 null mice had full reconstitution of all thymic subsets but the total number of donor thymocytes in the SP/Rag2 DKO was about 7-fold greater than in the Rag2 null mice.

### Example 8

### SPATIAL Induces Cell Growth Arrest

This example is a collection of several different approaches that collectively demonstrate that SPATIAL is a potent negative regulator of the cell cycle in a variety of different cell types.

### 1. Material and Methods

a. Cell Culture

Human embryonic kidney (293T) cells were maintained in DMEM supplemented with 10% heat-inactivated FBS, 2 mM glutamine, 100 U/ml penicillin, and 0.1 mg/ml streptomycin. Cells were grown at 37° C. in a humidified atmosphere with 5% CO₂.

The mouse thymocyte cell line Wehi 7.2 and its variant Wbcl-2, which over expresses bcl-2, were maintained in DMEM supplemented with 10% heat-inactivated FBS, 2 mM glutamine, 100 U/ml penicillin, and 0.1 mg/ml streptomycin. Cells were grown at 37° C. in a humidified atmosphere with 5% CO₂.

A suspension-adapted variant of the 293T cell line, called 293F (Invitrogen, Rockville, Md.), was maintained in FreeStyle 293 Expression Medium. Cells were shaken at all times on an orbital shaker in a 37° C. incubator with a humidified atmosphere of 8% CO₂.

b. Plasmid Construction

To create EGFP-SPATIAL(L) and EGFP-SPATIAL(S), the long and short isoforms of SPATIAL were cloned into the mammalian expression vector pEGFPN1 (Clontech, Palo Alto, Calif.) as Described in Flomerfelt et al., Genes and Immunity, 1:391-401, 2000. SPATIAL deletion mutants were created using EGFP-SPATIAL(L) and EGFP-SPATIAL(S) by restriction enzyme digestion followed by re-ligation. In some cases, linker oligonucleotides were used to preserve the reading frame.

c. DNA Transfections

Transient transfections were performed using either lipids (Lipofectamine 2000 or FreeStyle 293 Expression System, each supplied by Invitrogen, Rockville, Md.) or calcium phosphate (Mammalian Transfection Kit; Stratagene, La Jolla, Calif.) or electroporation. Manufacturer instructions were followed for lipid and calcium phosphate transfections. For electroporation, the following procedure was used: Before transfection, Wehi cell and Wbcl-2 cells were counted and suspended in fresh complete DMEM without antibiotics at 5×10⁵ cells/ml in a 15 cm dish. On the day of transfection, the cells were washed with PBS twice and were suspended in serum free DMEM at 6.25×10⁶ cells/ml. Aliquots of 800 μl cell suspension were transferred to a 4 mm electroporation cuvette and 10 μg of plasmid DNA solution was added. After 10 minutes incubation on ice, the cells were electroporated under the condition of 310 V, 725 ohms resistance, and 1050 μF capacitance. The transfected cells were incubated on ice for 10 minutes and suspended in complete DMEM in 10 cm dishes at the density of 7.5×10⁴ viable cells/ml. The cells were incubated at 37° C. in a CO₂ incubator.

Stable transfections were performed by Lipofectamine 2000.

d. Cell Growth Analysis

The day before transfection, the cells were trypsinized (if needed), counted and plated in a 24-well plate at the density of 7.5×10⁴ cells per well with 0.5 ml of their normal growth medium containing serum without antibiotics. On the day of transfection, cells were transfected according to the recommended protocol using 1.0 μg of each plasmid DNA and 3 μl of Lipofectamine 2000 in each well. The cells were kept at 37° C. in a CO₂ incubator for 24 hours. Cells from different wells transfected with the same DNA were trypsinized, pooled, split and plated to four 35 mm dishes. The number of EGFP-positive cells was calculated using fluorescence-activated cytometry (FACS) analysis at various time points after transfection.

e. Caspase Inhibition by Benzyloxycarbonyl-Valinyl-Alaninyl-Aspartyl Fluoromethylketone (Z-VAD.FMK)

The day before transfection, 293T cells were trypsinized, counted and plated at 2×10⁵ cells per well in 6-well plates. Two (2.0) ml of normal growth medium containing serum without antibiotics was added to each well. On the day of transfection, 6 wells of cells were pretreated for 4 hours with Z-VAD.FMK (BIOMOL Research Laboratories) (50 μM). A set of 6 wells of cells including 3 wells of Z-VAD.FMK-pretreated cells were transfected with 5 μg each of EGFP-SPATIAL(L) and the other set with 5 μg each of CD8-Flice-EGFP (Martin et al., J. Biol. Chem., 273(8):4345-4349, 1998), respectively. Three wells of cells were transfected with an expression plasmid for EGFP only (PEGFPN1; Clontech). These transfections were done by a calcium phosphate transfection method according to the recommended protocol. Transfection medium was replaced with fresh normal growth medium containing serum with antibiotics 7 hours after transfection. Cells were incubated at 37° C. in a CO₂ incubator for 17 hours. Then the cells were trypsinized, pooled, split and plated to four 60 mm dishes with 2.0 ml of their normal complete growth medium containing 50 μM Z-VAD.FMK. FACS analysis was used to obtain the number of EGFP-positive cells 24 and 48 hours after transfection.

f. Cell Cycle Analysis

293F cells were transfected with pEGFPN1 (Clontech), EGFP-SPATIAL(L) and EGFP-SPATIAL(S) as already described. EGFP-positive cells were sorted 48 and 120 hours after transfection. Then cell cycle analysis was carried out using propidium iodide as described by Lacana and D'Adamio (Nal. Med., 5(5):542-547, 1999).

### 2. SPATIAL Transfection Induces Cell Growth Arrest In Vitro

Efforts were made to produce stable SPATIAL transfections using a variety of cell lines, including 293T, 293HEK, NIH3T3 (mouse fibroblast cell line), COS7 (green monkey kidney cell line) and 427.1 (thymic subcapsular cortex or thymic nurse cell line described by Faas et al., Eur. J. Immunol., 23(6):1201-14, 1993). Approximately 0.5-1×10⁶ of each cell type was transfected with 1-10 μg of a plasmid that expressed either the SPATIAL(L) or (S) and a drug resistance gene. In all but 292T transfections, the drug resistance gene was the neo gene, which confers resistance to G418, an aminoglycosidic antibiotic that inhibits eukaryotic protein synthesis. For 293T transfections, the drug resistance gene was the hygromycin-resistance gene. Transfections were performed using Lipofectamine (Invitrogen) in accordance with the manufacturer's instructions.

Approximately 400 G418-resistant transfectants were screened for SPATIAL expression by RT-PCR or Western analysis. None of the G418-resistant transfectants expressed detectable amounts of SPATIAL. These results indicated that SPATIAL gene expression may be inconsistent with continued cell division necessary to obtain a stably transfected cell line.

Therefore, SPATIAL transient transfections were performed using at least each of the cell lines described above for stable transfections. Also included among cells examined by transient transfection were SAOS2 (human osteosarcoma) cells. Transient transfection of cells with SPATIAL resulted in profound growth inhibition in each cell line tested.

In representative experiments, approximately 0.5-1×10⁶ 293T cells were transiently transfected with approximately 1-10 μg of either SPATIAL-EGFP expression vector or EGFP expression vector by calcium phosphate precipitation using the Mammalian Transfection Kit (Stratagene) in accordance with the manufacturer's instructions. The number of fluorescent EGFP- or SPATIAL-EGFP-transfected cells was monitored over time using flow cytometry or microscopic examination.

Approximately 30-50% of EGFP- and SPATIAL-EGFP-transfected cells were measurably fluorescent 24 hours after transfection. This indicates that there was no substantial difference in transfection efficiency in the two cell populations. As shown in FIG. 6, at 48 hours post transfection, there was no significant difference in the number of EGFP-positive cells in the EGFP- and SPATIAL-EGFP-transfected samples. At four days post-transfection, approximately 60-80% of EGFP-transfected cells were fluorescent, which indicates these cells were growing and dividing as expected (see FIG. 6). In contrast, there was no increase in the number of fluorescent cells in the SPATIAL-EGFP-transfected sample over the same four day period (see FIG. 6). This indicates that transfection of SPATIAL-EGFP resulted in a population of fluorescent cells that did not grow normally. Similar results were obtained using other epithelial, fibroblast or lymphoid cell lines.

293T cells were also transiently transfected with either SPATIAL-EGPF/neo or EGFP/neo expression plasmids as described previously in this example except that G418 selection was used from the date of transfection onward to eliminate untransfected cells. Over a two-week period post-transfection, the number of fluorescent EGFP-transfected cells increased by more than 100 fold, while the number of fluorescent SPATIAL-EGFP transfected cells increased only 2-3 fold.

Both SPATIAL isoforms induced the same level of growth arrest in each experiment described in all of the foregoing transfection experiments.

### 3. SPATIAL-induced Growth Arrest is not Due to Toxic Accumulation of SPATIAL-EGFP

It is known that EGFP is extremely stable and confers stability to fusion proteins (Li et al., J. Biol. Chem., 273(52):34970-34975, 1998). To address whether the growth arrest in SPATIAL-EGFP transfected cells was due to toxic accumulation of SPATIAL-EGPF, SPATIAL-d2EGFP fusion constructs were produced. The d2EGFP protein is a modified form of EGFP with a two hour half-life (Li et al., J. Biol. Chem., 273(52): 34970-34975 1998).

The SPATIAL long and short isoforms were excised from SPATIAL-EGFP expression vectors using Nhe1 and Age1 restriction enzymes. The purified cDNA from the digestions was ligated into the corresponding restriction sites in pD2EGFP-N1 (Clontech).

Substitution of SPATIAL-d2EGFP for SPATIAL-EGFP in transfections such as those described above did not affect the growth inhibition of transfected SPATIAL fusion constructs. The only difference was a decrease in mean fluorescence of individual cells when d2EGFP was used.

### 4. Mapping the Growth-Regulatory Region of SPATIAL

To identify what portion of the SPATIAL protein mediates growth suppression, a panel of SPATIAL-EGFP deletion mutants (as shown in Table 3) was created using molecular cloning methods well known in the art. The nucleic acid sequence of each mutant was verified by restriction analysis and sequencing.

TABLE 3

SPATIAL Deletion Mutations

| | | |
|---|---|---|
| Construct | Nucleotide Deletion | Amino Acid Deletion |

| | | |
|---|---|---|
| SP-Delta SB5 | 145-677^{a} | 21-197^{c} |
| | 145-779^{b} | 21-231^{d} |
| SP-Delta XB | 354-677^{a} | 91-197^{c} |
| | 354-779^{b} | 91-231^{d} |
| SP-Delta M12 | 354-611^{b} | 91-176^{d} |
| | (with nucleotides | (with amino acids “NRFA” inserted |
| | “AACCGGTTCGCG” added after | in the deleted region) |
| | nucleotide 353) | |
| SP-S-Delta B1 | 516-677^{a} | 145-197^{c} |
| SP-L-Delta B2 | 618-779^{b} | 178-231^{d} |
| SP-Delta NX | 1-344^{b} | 1-97^{d} |
| | (with nucleotides “ATGTTC” added | (with amino acid “MF” added at the |
| | to 5′ end of deletion mutant) | N-terminus of the mutant) |

^{a}Nucleotide positions correspond to those set forth in SEQ ID NO: 1 (SPATIAL(S))
^{b}Nucleotide positions correspond to those set forth in SEQ ID NO: 3 (SPATIAL(L))
^{c}Amino acid positions correspond to those set forth in SEQ ID NO: 2 (SPATIAL(S))
^{d}Amino acid positions correspond to those set forth in SEQ ID NO: 4 (SPATIAL(L))

Equal numbers of cells were plated and transfected with pEGFPN1 (Clontech), EGFP-SPATIAL(L), or a SPATIAL deletion mutant. Seventy-two (72) hours post transfection, cells were harvested, counted, and the percent of EGFP-positive cells were determined using a flow cytometer.

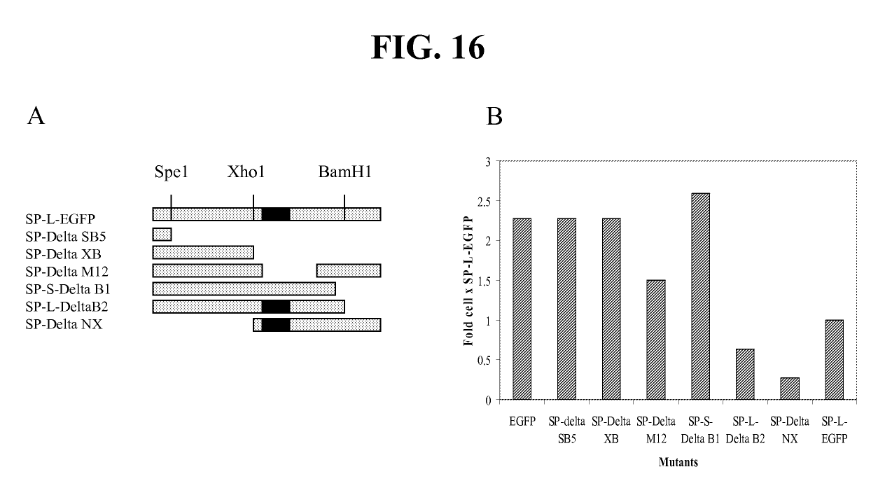

FIG. 16 shows the number of EGFP-positive cells for each construct relative to the number of EGFP-positive cells for wild type EGFP-SPATIAL(L). As expected a nearly complete deletion, SP-Delta SB5, had little growth suppression capacity. The same result was seen in deletions SP Delta XB and SP-S Delta B1. Removal of the portion of the gene shown in SP-Delta M12 resulted in partial growth suppression. In contrast, removal of the N-terminus (SP-Delta NX) or the C-terminus of the longer isoform of SPATIAL (SP-L-Delta B2) had no effect on growth suppression.

Taken together, this data suggests that the portions of SPATIAL between the Xho1 and BamH1 sites (as shown schematically in FIG. 16) and the carboxyl terminus are associated with a growth inhibitory activity of SPATIAL. Since both isoforms are equally effective in mediating growth suppression, the alternatively spliced exon alone is not sufficient for SPATIAL's growth inhibitory activity. However, removal of carboxyl-terminal amino acids eliminates growth suppression of the short (SP-S-Delta B1), but not the long isoform (SP-L-Delta B2). Removal of the carboxyl half of the long isoform (SP-Delta XB) results in a complete loss of growth suppression while the removal of the N-terminal half of SPATIAL (SP-Delta NX) has little effect. A partial loss of growth suppression is seen when amino acids 91 to 176 (SP-Delta M12) are removed from the long isoform. This deletion includes amino acids surrounding the alternatively spliced exon.

These results suggest that two separable regions are involved in mediating SPATIAL-induced growth suppression. One of these is found within the C-terminal 53 amino acids while a second is found in region surrounding and including the alternatively spliced exon.

5. SPATIAL does not Induce Apoptosis

One explanation for the SPATIAL-induced growth arrest described in this Example 8 is that SPATIAL causes cells to undergo apoptosis. Three different assays described in this example demonstrate that SPATIAL does not induce apoptosis. Consequently, it should be noted that it is possible to isolate and analyze viable cells transiently transfected with SPATIAL. Up to 5×10⁶ SPATIAL-EGFP transfected cells have been successfully isolated 168 hours after transfection using fluorescent cell activated sorting. These procedures can be scaled up to obtain as many cells as may be needed for a particular purpose.

a. Propidium Iodide Staining

Propidium iodide is a commonly known fluorescent compound, which intercalates into double-stranded nucleic acids. It is excluded by viable cells but can penetrate cell membranes of dying or dead cells. Thus, apoptotic cells may be identified by staining (and, in particular, nuclear staining) with propidium iodide.

Approximately 0.5-1.0×10⁶ 293T cells were transfected with SPATIAL-EGFP using either calcium phosphate (Mammalian Transfection Kit; Stratagene, La Jolla, Calif.) or Lipofectamine (Invitrogen, Rockville, Md.) in accordance with the manufacturers' instructions. Twenty-four (24) to 72 hours after transfection, the transfected cells were sorted. Approximately 1-5×10⁵ sorted cells were resuspended in a solution contain 50 μg/ml propidium iodine, 0.1% sodium citrate and 0.1% Triton X-100 and incubated overnight to lyse the cells and stain the nuclei. Then, the sample was analyzed by flow cytometry to identify sub-diploid amounts of DNA which indicates cell death via apoptosis.

There was no evidence of apoptosis based on nuclear morphology of propidium iodide stained SPATIAL-EGFP transfected cells.

b. Caspase Activation

Caspase activation plays a central role in the execution of apoptosis (e.g., Budihardjo et al., Ann. Rev. Cell. Dev. Biol., 15:269-90, 1999). No caspase activation was detected using a sensitive flow cytometry assay (PhiPhi Lux, OncoImmunnin, Inc., College Park, Md.) in SPATIAL-EGFP transfected cells.

Further, 293T cells were treated with a known pan-caspase inhibitor, Z-VAD FMK, and transfected with an expression plasmid that encodes a CD8⁻ Flice-EGFP fusion protein, which efficiently induces apoptosis (Martin et al., J. Biol. Chem., 273(8):4345-4349, 1998). The amount of Z-VAD FMK expression plasmid needed to block CD8⁻ Flice-EGFP-induced apoptosis was determined.

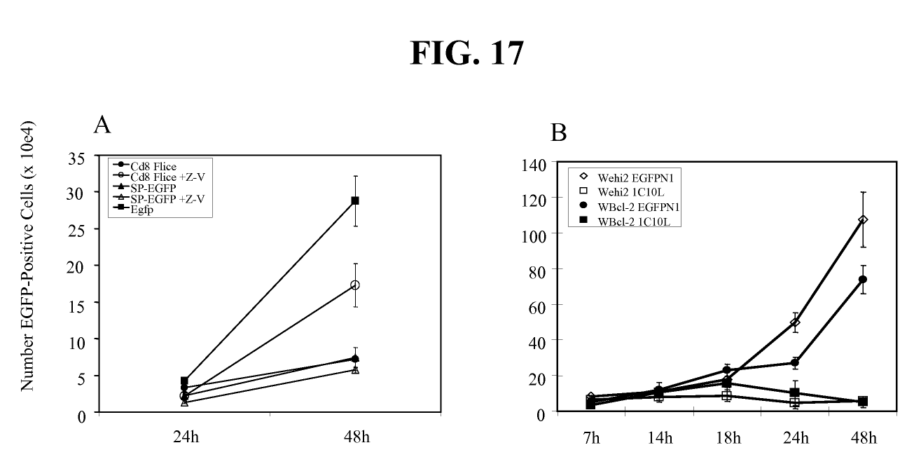

As shown in FIG. 17 A, the concentration of Z-VAD FMK that was sufficient to block CD8⁻ Flice-EGFP-induced apoptosis did not affect the SPATIAL-induced block in cellular growth.

c. Bcl-2 Expression

Expression of Bcl-2 is known to inhibit apoptosis in a variety of experimental systems (Flomerfelt and Miesfeld, J. Cell. Biol., 127(6):1729-1742, 1994). However, Bcl-2 expression was ineffective to reverse the growth suppressive effect of SPATIAL-EGFP transfection.

Two thymocyte cell lines, Wehi7.2 and Hbl2, were used in this example. Wehi7.2 cells undergo apoptosis in response to a broad range of stimuli (including, glucocorticoids) (Flomerfelt and Miesfeld, J. Cell. Biol., 127(6):1729-1742, 1994). In contrast, Hb12 cells, which are stable transfectants of Wehi7.2 cells that expresses human Bcl-2 gene, are resistant to apoptosis (Flomerfelt and Miesfeld, J. Cell. Biol., 127(6):1729-1742, 1994). Approximately 1×10⁶ of each of Wehi7.2 and Hb12 cells were transiently transfected with SPATIAL-EGFP by electroporation, and the growth characteristics of the SPATIAL-EGFP transfectants were observed.

SPATIAL-EGFP-transfected Wehi 7.2 cells and Hb12 cells each exhibited profound suppression of cell growth similar to that described for 293T cells in this Example 8. If SPATIAL-EGFP induced growth suppression occurred through an apoptotic mechanism, one would have expected only SPATIAL-EGFP-transfected, apoptosis-sensitive Wehi7.2 cells, not apoptosis-resistant Hbl2 cells, to be growth suppressed.

Dexamethasone (1 μM), a synthetic glucocorticoid, added to Wehi7.2 and Hb12 SPATIAL-EGFP-transfected cultures induced apoptosis in transfected Wehi7.2 cells but not in transfected Hbl2 cells. Hence, the apoptotic pathway was intact in SPATIAL-EGFP-transfected Wehi7.2 cells.

Taken together, the results of the above-described propidium iodide staining, caspase activation, and Bcl-2 expression assays demonstrate that apoptosis is not a factor in the lack of growth in SPATIAL-EGFP transfected cells.

### 6. SPATIAL Affects Cell Cycle Control

Another explanation for the SPATIAL-induced growth arrest described in this Example 8 is that SPATIAL affects a cell cycle control mechanism. This example demonstrates that SPATIAL causes cells to be arrested in the G1 phase of the cell cycle.

a. SPATIAL Transfection Results in a Block in Cell Division

Since SPATIAL does not appear to induce apoptosis, the effect of SPATIAL transfection on cell division was determined. To directly examine division of SPATIAL-EGFP transfected cells, the lipophilic dye, 3H-Indolium, 2-[3-(1,3-dihydro-3,3-dimethyl-1-octacdecyl-2H-indol-2-ylidene)-1-propenyl]-3,3-dimethyl-1-octacdecyl-, perchlorate (DiI). DiI becomes highly fluorescent when it interacts with cell membranes and is equally portioned to each daughter cell during cell division. This property has been used to monitor cell divisions based on the reduction of dye in successive generations, which can be monitored using FACS analysis (Huang et al., Blood, 94(8):2595-2604, 1999). DiI was chosen so that the generational analysis could be done on EGFP-positive cells.

An analysis of the cell divisions of EGFP-positive cells is shown in FIG. 7. As shown in the figure, transfection with EGFP alone did not block cell division over a 4-day period. In contrast, expression of SPATIAL-EGFP effectively blocked the majority of transfected cells from dividing at all, while a small percentage of cells did divide once. Analysis of EGFP-negative cells in the SPATIAL-EGFP-transfected culture showed that the EGFP-negative cells were dividing normally indicating that the cell division block was specific to cells expressing SPATIAL-EGFP.

b. SPATIAL Transfection Blocks Exit from the G1 Phase

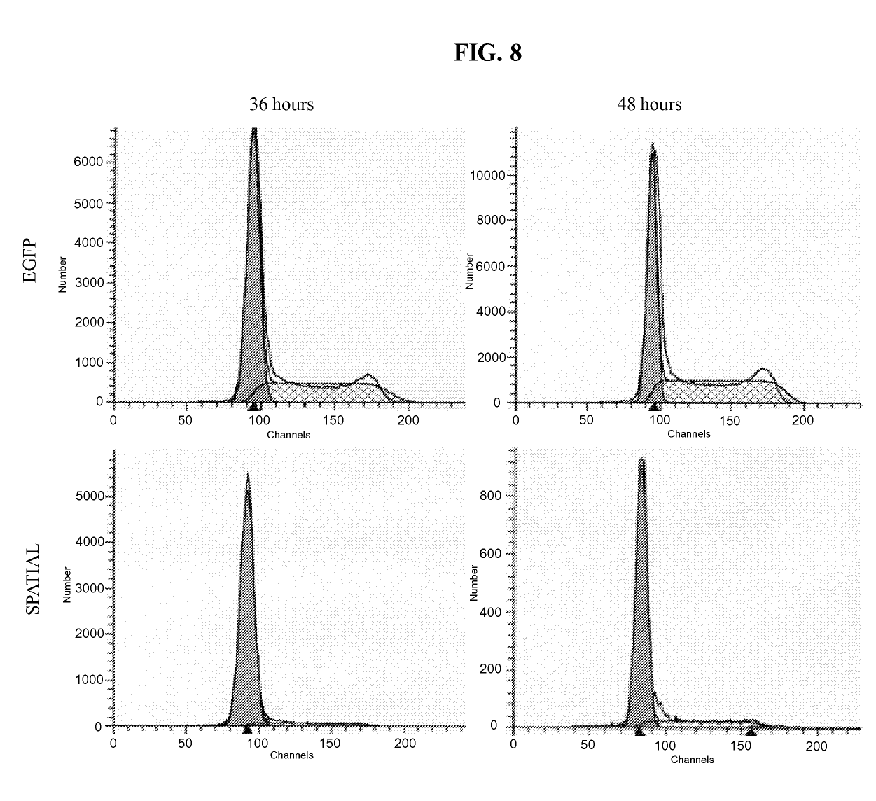

To determine what stage of the cell cycle was blocked, SPATIAL-EGFP or EGFP alone (PEGFPN1; Clontech) was transfected into NIH 3T3 cells. EGFP-positive cells were sorted at various times and DNA content was measured in 1-2×10⁵ isolated nuclei to obtain a cell cycle profile of the population. As shown in FIG. 8, transfection with SPATIAL resulted in a decrease in the number of cells that entered S phase compared to those cells transfected with EGFP.

Collectively, this Example 8 demonstrates that SPATIAL plays a role in the control of cell cycle progression. This effect occurs in a wide variety of different cell types, including, for example, epithelial, (293T, 293HEK, 427.1), fibroblast (NIH3T3), and lymphoid (Wehi7.2) cells. Therefore, SPATIAL appears to operate through a conserved cell cycle control mechanism. In addition, this mechanism appears to be quite potent as it operates in transformed cell lines such as 293T which are known to be defective in a number of cell cycle control pathways and to express multiple oncogenic proteins (Numa et al., Cancer Res., 55:4676-4680, 1995)

### Example 9

### SPATIAL Specifically Interacts with Uba3

This example demonstrates that SPATIAL is involved in protein-protein interactions with proteins expressed in the day-18 mouse embryo. In particular, SPATIAL interacts with Uba3.

### 1. Yeast Two-Hybrid System

A yeast two hybrid screen was performed using the BD Matchmaker™ Mammalian Two-Hybrid Assay Kit 2 (Clontech) in accordance with manufacturer's instructions to identify proteins that interact with SPATIAL. A full-length cDNA of the long form of SPATIAL was fused to the Gal4 DNA binding domain construct and was used as the bait. Prey constructs were obtained from a day-18 mouse embryo library purchased from Clontech. The day-18 embryo has a fully functional thymus that expresses SPATIAL.

Putative interacting proteins were identified by auxotrophic growth on media lacking histidine, leucine, tryptophan, and adenine. Lamin C was used as an irrelevant control to test putative interactor proteins for specific binding to SPATIAL in yeast. The cDNAs from yeast clones that exhibited specific interaction with SPATIAL were isolated, sequenced, and recloned into different expression vectors.

Three of the cDNAs identified in the yeast two-hybrid system encoded some portion of Uba3 (a.k.a., catalytic subunit of NEDD8 activating enzyme), a 441-amino acid protein (SEQ ID NO: 6) involved in cell cycle regulation. The largest cDNA (clone 346; residues 646 to 1365 of SEQ ID NO: 5) encoded 235 amino acids of the carboxyl terminus of Uba3 (corresponding to residues 203 to 441 of SEQ ID NO: 6).

### 2. GST-Pulldown Assay

Eighteen cDNA clones identified in the yeast two-hybrid system were tested in an in vitro glutathione-S-transferase (GST) pulldown assay, as described in Ausubel et al., Current Protocols in Molecular Biology, Greene Publishing Associates, updated November 2003, Chapter 20, Analysis of Protein Interactions, Unit 20.2, Affinity Purification of Proteins Binding to GST Fusion Proteins.

Briefly, a GST-SPATIAL fusion construct was produced by cloning SPATIAL cDNA(s) into pGEx5×1 expression vector using standard molecular cloning techniques. GST or GST-SPATIAL fusion protein were expressed in 1-1000 ml bacterial cultures grown for 3-4 hours until OD₆₀₀ reached 0.5. Then, plasmid expression was induced for 1.5-4 hours by the addition of ITPG to a final concentration of 0.1 mM. GST or GST-fusion proteins were purified using glutathione-Sepharose beads as described in Ausubel et al., Current Protocols in Molecular Biology, Greene Publishing Associates, updated November 2003, Chapter 20, Analysis of Protein Interactions, Unit 20.2, Affinity Purification of Proteins Binding to GST Fusion Proteins.

A coupled in vitro transcription and translation system (Promega) was used according to the manufacturer's instructions to produce radiolabeled protein from cDNAs to be tested. Radiolabeled proteins were mixed with either purified GST or GST-SPATIAL. Then, GST-containing protein complexes were purified by centrifugation from the reaction mixtures using glutathione-coated Sepharose beads. Bound proteins were eluted from the beads with SDS-PAGE loading buffer heated to 72-85° C., separated on polyacrylamide gels, and visualized by autoradiography. Specific interactions between GST-SPATIAL and in vitro translated proteins were indicated by the presence of a radiolabeled in vitro translated protein in the GST-SPATIAL-containing samples, but not in the GST-alone samples.

As shown in FIG. 3, in vitro translated protein produced from Myc-tagged Uba3-clone 346 (Myc-346; lane “IVT”) specifically interacted with GST-SPATIAL(L) fusion protein (lane “GST-SPATIAL(L)”) but not with GST alone (lane “GST”). FIG. 3, lane “IP” is a positive control showing that Myc-346 is specifically immunoprecipitated by an anti-Myc antibody added to the IVT reaction mixture.

### 3. Co-Immunoprecipitation of SPATIAL and Uba3

Expression vectors encoding Myc-346 and HA-tagged SPATIAL were transfected alone or in combination into 293T cells by calcium phosphate precipitation (Mammalian Transfection Kit; Stratagene, La Jolla, Calif.) in accordance with manufacturer's instructions. Total protein lysates were prepared and immunoprecipitated with an anti-HA antibody, as described in Ausubel et al., Current Protocols in Molecular Biology, Greene Publishing Associates, updated November 2003, Chapter 20, Analysis of Protein Interactions, Unit 20.5, Detection of Protein-Protein Interaction by Coprecipitation. The immunoprecipitated proteins were separated by SDS-PAGE and transferred to a membrane for Western blotting using an anti-Myc antibody.

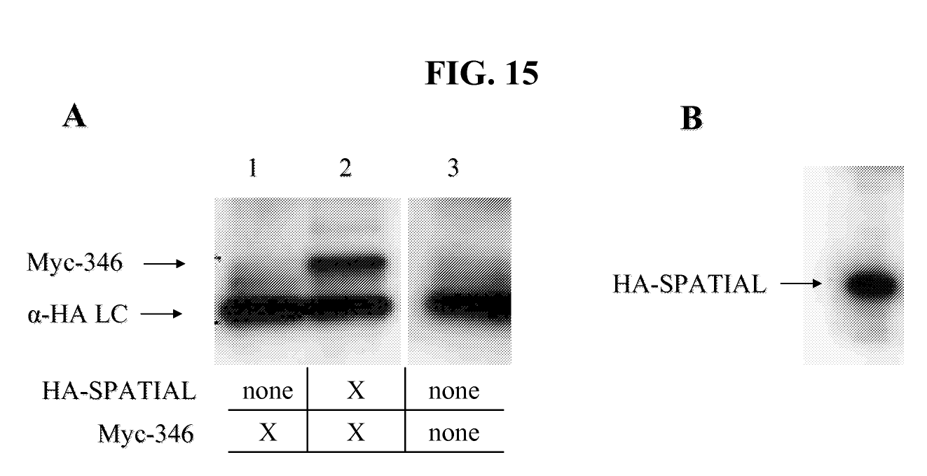

As shown in FIG. 15, Myc-346 was reproducibly immunoprecipitated by anti-HA antibody only in samples that contained HA-tagged SPATIAL.

This example demonstrates genetic and physical evidence from three independent experimental systems showing a specific protein-protein interaction between SPATIAL and Uba3.

### Example 10

### Mapping of Regions Involved in SPATIAL/Uba3 Interaction

This example demonstrates that amino acid residues 183 to 308 of Uba3 (SEQ ID NO: 6) influence and/or are involved in the protein-protein interaction between SPATIAL and Uba3.

Several Uba3 deletion mutations were made was created using molecular cloning methods well known in the art.

Table 4 identifies the nucleotide and corresponding amino acid deletions that were made in the Uba3 deletion constructs. In addition, a construct consisting of nucleotides 586-963 of Uba3 (SEQ ID NO: 5) (corresponding to amino acid residues 183 to 308 of SEQ ID NO: 6) was constructed by amplifying the desired fragment from the full-length Uba3 sequence using primers complementary to the desired sequences (which primers also contained, as appropriate, restriction sites suitable for cloning the amplified fragment into a desired expression vector(s), e.g., pcDNA3.1 myc/His (Invitrogen), and an ATG codon with a KOZAK sequence).

TABLE 4
Uba3 Deletion Mutations

| Construct | Nucleotide Deletion¹ | Amino Acid Deletion² |
|---|---|---|
| Delta24-646 | 24-646 | 1-202 |
| Delta941-1365 | 941-1365 | 301-441 |
| Delta364-912 | 364-912 (PstI digest) | 109-291 |
| Delta348-431 | 348-431 | 103-130 |
| Delta451-552 | 451-552 | 138-171 |
| Delta579-623 | 579-623 | 181-195 |
| Delta643-705 | 643-705 | 202-222 |
| Delta728-763 | 728-763 | 231-242 |
| | | (with E230A substitution) |
| Delta784-825 | 784-825 | 249-262 |
| Delta844-885 | 844-885 | 269-282 |

¹Nucleotide positions correspond to those set forth in SEQ ID NO: 5
²Amino acid positions correspond to those set forth in SEQ ID NO: 6

GST pulldown assays were performed as described in Example 9 using GST-SPATIAL(L) and GST-SPATIAL(S) and each of the Uba3 mutants described in this example.

As shown in FIG. 5, both isoforms of SPATIAL interact with Uba3 deletion mutants that lack the amino and carboxyl terminus of the protein (Delta24-646 and Delta941-1365, respectively). However, the Delta364-912 mutant (from which the PstI fragment of the Uba3 coding sequence has been deleted) does not interact with either SPATIAL isoform. This finding demonstrates that the central portion of the Uba3 sequence is involved in the Uba3/SPATIAL interaction. Indeed, a fragment of Uba3 encoded by nucleotides 386-963 of Uba3 (corresponding to residues 586-963 of SEQ ID NO: 5) retains the ability to interact with both GST-SPATIAL(L) and GST-SPATIAL(S).

Therefore, at least amino acids 183 to 308 (corresponding to nucleotides 586-963) of Uba3 (SEQ ID NOs: 5 and 6, respectively) are involved in the protein-protein interaction with SPATIAL.

A panel of smaller Uba3 deletion mutants (10-20 amino acid deletions), which are also identified in Table 4, have been created using site directed mutagenesis and will be useful to further dissect which residues of Uba3 that are involved in the SPATIAL/Uba3 interaction.

### Example 11

### SPATIAL Disrupts an Interaction Between Uba3 and AppBP1

This example demonstrates that SPATIAL specifically binds Uba3 and disrupts the binding between Uba3 and AppBP1. Thus, SPATIAL is believed to inhibit the neddylation pathway and inhibit cells from dividing.

Uba3 is the catalytic subunit of the activating enzyme in the NEDD8 conjugation (neddylation) pathway (Gong and Yeh, J. Biol. Chem., 274(17):12036-12042, 1999). The neddylation pathway is conserved among yeast and other eukaryotes, including mice and humans. Activation of the neddylation pathway results in the degradation of kinase inhibitory proteins which otherwise stop the cell from entering S phase and dividing (Podust et al., Proc. Natl. Acad. Sci., 97(9):4579-4584, 2000). Hence, activation of the neddylation pathway by Uba3-containing NEDD8 activating enzyme permits cells to divide. In mice, deletion of Uba3 is lethal and blocks the NEDD8 pathway which inhibits cell cycle progression of the preimplantation embryo (Tateishi et al., J. Cell Biol., 155(4):571-579, 2001).

Other subunits of the neddylation pathway activating enzyme include AppB1 and Ubc12 (Gong and Yeh, J. Biol. Chem., 274(17):12036-12042, 1999). Uba3 and AppBp1 are known to form a protein-protein interaction (Gong and Yeh, J. Biol. Chem., 274(17):12036-12042, 1999). The effect of SPATIAL on the Uba3 and AppB1 interaction was examined in this example.

To determine whether SPATIAL has an effect on the neddylation pathway, cDNAs for each of the components of the Nedd8 conjugation pathway were obtained from EST depositories (AppBP1, GenBank Accession No. BC00480; Uba3, GenBank Accession No. BC002002). These cDNAs were used to produce radiolabeled proteins by in vitro transcription and translation.

The effect of SPATIAL on the first step of the pathway, the interaction between Uba3 and AppBP1, was tested using a GST pulldown assay

In a typical GST pulldown assay, GST and GST-SPATIAL were prepared from transformed bacterial cultures as previously described (e.g., Ausubel et al., Current Protocols in Molecular Biology, Greene Publishing Associates, updated November 2003, Chapter 20, Analysis of Protein Interactions, Unit 20.2, Affinity Purification of Proteins Binding to GST Fusion Proteins). After purification, GST and GST-SPATIAL proteins were mixed with glutathione-sepharose beads and the mixtures were frozen in 200 μl aliquots. The amount of protein bound to the beads was quantitated using a colorimetric assay (BCA assay; Pierce-Endogen).

Uba3 and AppBP1 were in vitro translated and ³⁵S-methionine labeled using 0.5 μg of the respective expression plasmids in a 50 μl reaction using TNT® Coupled Reticulocyte Lysate Systems (Promega) by following the manufacturer's instructions. The radiolabeled proteins were diluted 5 fold with the addition of phosphate buffered saline (PBS). Then, 25 μl of GST or GST-SPATIAL (long and/or short isoforms) bound to glutathione-sepharose beads was added to 25 μl of the mixture of radiolabeled in vitro translated Uba3 and AppBP1 overnight at 4° C. with rotation. The mixture was then separated by centrifugation and the precipitate (including the glutathione beads and bound proteins) was separated from the supernatant. The beads were washed 5 times with 1 ml of PBS-Tween to remove unbound proteins. SDS-PAGE gel buffer and 2-β-mercaptoethanol was added to the pelleted beads and the samples were heated at 72° C. for 10 minutes. The pelleted gel sample was clarified by centrifugation, and the supernatant separated by SDS-PAGE.

For immunoprecipitations, an in vitro translated mixture containing ³⁵S-methionine-labeled, Myc-tagged Uba3 and AppBP1 was incubated for 1-4 hours at 25° C. Then, 1-5 μl of anti-Myc antibody (0.5 mg/ml) was added to the mixture for approximately 12 hours at 4° C. with rotation. Twenty-five (25) to 50 μl of 50% Protein A sepharose slurry (Amersham-Pharmacia) was then added to the mixture and incubated at 4° C. for 1-2 hours. The mixture was then centrifuged to pellet the beads and the supernatant removed. The beads were washed extensively with TBS-Tween solution (100 mM Tris Cl, pH 7.5, 150 mM NaCl, 0.1% Tween 20), and the bound proteins were eluted from the beads with 20 μl SDS-PAGE loading buffer at 70-85° C.

FIG. 4A shows that ³⁵S-methionine-labeled, Myc-tagged Uba3 and a ³⁵S-methionine-labeled, Myc-tagged Uba3/AppBP1 protein complex is immunoprecipitated from an in vitro translated mixture of Myc-tagged Uba3 and AppBP1. Thus, the Uba3/AppBP1 complex is formed under the conditions described in this example.

FIGS. 4B and 4C show the distribution of radiolabeled Uba3 in the precipitate and supernatant, respectively, of a GST pulldown assay. Substantial Uba3 is present in the precipitate, which demonstrates that Uba3 specifically bound GST-SPATIAL and was pulled down on the glutathione-coated beads. As shown in FIG. 4B, AppBP1 was not able to bind Uba 3 complexed with SPATIAL. Since excess radiolabeled proteins were present in the GST pulldown assay, Uba3 and AppBP1 were present in the supernatant after the GST-pulldown assay. Using an anti-Myc antibody to immunoprecipitate the excess Uba3 from the supernatant of the GST pulldown assay, FIG. 4C shows that Uba3 not complexed with SPATIAL was able to complex with AppBp1 (compared FIG. 4C with FIG. 4A).

Collectively, these findings demonstrate that GST-SPATIAL specifically binds to Uba3 and interferes with Uba3 binding to AppBP1. Thus, SPATIAL can prevent the first step on the Nedd8 conjugation pathway and is expected to block events downstream in that pathway.

### Example 12

### Uba3 Overcomes SPATIAL-Induced Growth Arrest In Vitro

To determine whether Uba3 is involved in SPATIAL-mediated growth suppression, co-transfections were done using increasing amounts of an Uba3 expression vector with constant amounts of SPATIAL-EGFP or EGFP.

4×10⁵ 293T cells were transfected in 6 well plates using 5 μg of a mixture of plasmids. The plasmids used were pEGFP-N1 (0.5 μg/well), pEGFP-SPATIAL(L) (0.5 μg/well), pcDNA3.1 Myc/his M Uba3 (0.5, 2.5 or 4.5 μg/well) (pcDNA3.1 myc/His may be obtained from Invitrogen), and pWL-neo (4 or 2 μg/well). The plasmid pWL-neo (Stratagene) was used as a control plasmid to make sure that the total DNA in each transfection totaled 5 μg. Transfections were done using Lipofectamine 2000 using the manufacturer's instructions (Invitrogen).

Twenty four hours after transfection, the cells were trypsinized and re-plated onto 10 cm dishes and cultured for up to 6 days. The number of EGFP-positive cells were determine at 24, 36 and 144 hours after transfection by harvesting the cells, counting and FACS analysis to determine the percentage of EGFP-positive cells.

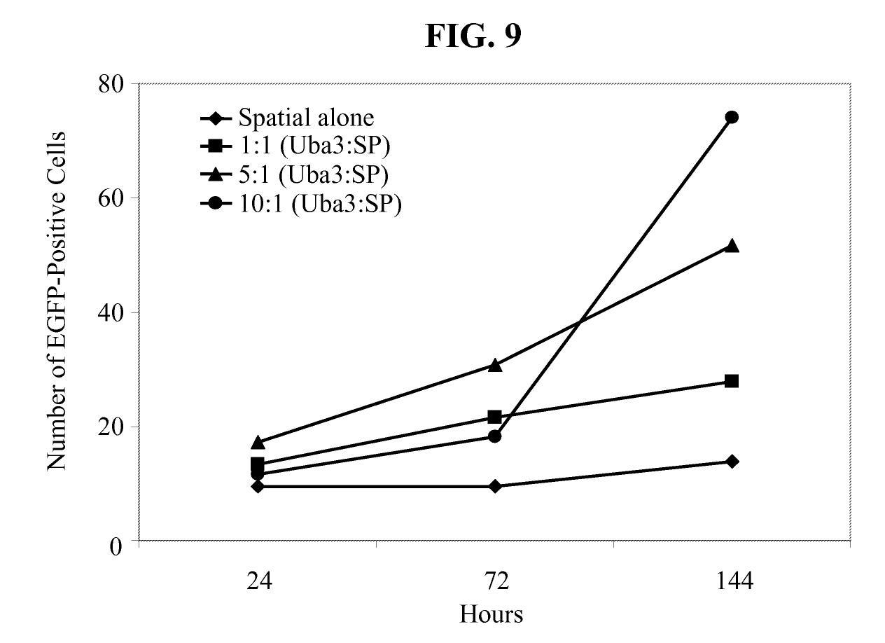

As shown in FIG. 9, transient over expression of Uba3 overcomes the growth inhibition of SPATIAL-EGFP transfected cells. This result indicates that SPATIAL-dependent growth arrest is mediated through Uba3.

### Example 13

Identification of Anti-Sense SPATIAL Oligonucleotides

This example describes a representative method useful for identifying SPATIAL anti-sense phosphorothioate chimeric oligonucleotides (PSC-oligos), which can specifically decrease SPATIAL expression in the thymus. PSC-oligos are useful for anti-sense treatment because of their long term stability in cells, increased target specificity, and low toxicity (for example, LD50 in mice is 500 mg/kg, while effective doses occur at 5-10 mg/ml) (see, e.g., Agrawal and Zhao, Curr. Opin. Chem. Biol., 2(4):519-528, 1998).

RNAseH is a nuclease that recognizes and specifically degrades the RNA strand in an RNA:DNA duplex. RNAseH mapping can identify DNA oligonucleotides that anneal to a target RNA (see, e.g., Ho et al., Nucleic Acids Res., 24(10):1901-1907, 1996). Briefly, the procedure involves using a defined RNA (such as, SPATIAL RNA), which is produced in vitro, as a target to identify those oligonucleotides that specifically bind the target RNA (for example, SPATIAL RNA) from a pool of oligonucleotides (also referred to as an oligonucleotide library).

A reverse phase HPLC purified random library of PSC-oligos of defined length is produced using a mixture of phosphoramidates to synthesize oligonucleotides (Touleme et al., Prog. Nucleic Acid Res. Mol. Biol., 69:1-46, 2001). A full-length SPATIAL RNA target is synthesized using a large-scale in vitro transcription assay and purified (Flomerfelt et al., Genes Immun., 1:391-401, 2000). The SPATIAL RNA and random PSC-oligo library are mixed and allowed to hybridize under conditions empirically determined to allow specific interactions as described in Ho et al. (Nucleic Acids Res., 24(10):1901-1907, 1996). Then, RNAseH is added to the RNA/oligonucleotide mixture and the digestion reaction proceeds for approximately 30 minutes. The reaction is stopped by the addition of RNAse inhibitors and EDTA, and the resulting RNA fragments are reverse transcribed into corresponding cDNA fragments using several different C-terminal SPATIAL-specific primers. This results in a population of cDNAs having the 5′ end of each molecule specific for a primer sequence that mediated in vitro RNAse cleavage. The cDNA fragments are separated on a denaturing polyacrylamide gel. A sequencing gel of the full-length SPATIAL cDNA using the same primer is also produced. The sequences of anti-sense PSC-oligos that specifically bind SPATIAL RNA is determined by observing where the bands line up on the two gels.

PSC-oligos identified in this example may be used to inhibit SPATIAL gene expression, for instance, in vitro and in vivo as described in the following examples.

### Example 14

### In Vitro Inhibition of SPATIAL Expression Using Anti-Sense Oligonucleotides

In this example, PSC-oligos identified in Example 13, or by other means, are used to inhibit SPATIAL expression in a tissue culture system.

To facilitate uptake of oligonucleotides by cells in culture, Lipofectamine (Invitrogen) is used to transfect both anti-sense oligonucleotides and expression plasmids. Control oligonucleotides will be prepared using the same nucleotides as the SPATIAL-specific anti-sense oligonucleotides but in scrambled order such that the overall nucleotide composition of the control is the same but the control oligonucleotides will not bind to SPATIAL mRNA. A series of transient transfections are performed using 293T cells, which can be transfected at a high frequency, using a constant amount of SPATIAL-EGFP or EGFP expression plasmids and increasing amounts of control or anti-sense PSC-oligos.

Twenty-four hours after transfection, the cells are analyzed by flow cytometry to count the number and intensity of fluorescent cells. Anti-sense inhibition is indicated by a dose-dependant decrease in the number and intensity of SPATIAL-EGFP-transfected fluorescent cells co-transfected with a particular anti-sense oligonucleotide as compared to EGFP-transfected fluorescent cells treated with the same oligonucleotide.

### Example 15

### In Vivo Inhibition of SPATIAL Expression Using Anti-Sense Oligonucleotides

This example describes representative methods of inhibiting SPATIAL gene expression in vivo, and describes how to obtain a dose of anti-sense PSC-oligos useful for a thymic conditioning treatment prior to BMT.

Rag2 DKO mice can serve as immunodeficient hosts to receive the anti-sense SPATIAL oligonucleotides following BMT. An osmotic pump (for example, DURECT Corporation, Cupertino, Calif.) is used to automate delivery of anti-sense oligonucleotides to the subjects. This delivery method reduces animal handling and the stress that may accompany administration of the anti-sense oligonucleotides by multiple injections. Moreover, an osmotic pump is useful for ease of maintaining steady bioactive levels of the anti-sense oligonucleotides in the thymus. Alternatively, the anti-sense oligonucleotides can be injected intravenously, intramuscularly, or directly into the thymus. In one embodiment, the anti-sense oligonucleotides are injected intravenously into the tail vein or intraperitoneally.

The osmotic pump is loaded with either anti-sense or control (scrambled anti-sense sequence, as described in Examples 13 and 14) PSC-oligos. The osmotic pump is implanted into mice anesthetized with 0.1 mg/gm ketamine (anesthetic) and 0.002 mg/gm xylazine (muscle relaxant). Hair is shaved and the skin disinfected. Typically, a 10 mg/ml ketamine, 0.2 mg/ml xylazine sterile stock solution is prepared and 0.2 cc-0.3 cc is injected intraperitoneally. A small incision is made in the loose skin on the back, the pump is inserted and the wound is clipped shut. (See, e.g., Ghirnikar and Lee, Neurosci. Lett., 247(1): 21-24, 1998.) “Naked” PSC-oligos can be administered because their uptake is efficient in vivo (e.g., Akhtar et al., Adv. Drug Deliv. Rev., 44:3-21, 2000). Alternatively, the anti-sense oligonucleotides may be coupled to peptides or antibodies or administered with additives (such as lipids, polymers, or nanoparticles) to enhance their uptake (as described, e.g., by Agrawal and Zhao, Curr. Opin. Chem. Biol., 2(4):519-528, 1998).

Toxicity is determined by titrating the subject mice with 1 mg/kg to 250 mg/kg of anti-sense oligonucleotide. Veterinarians monitor the overall health of the mice during the course of treatment. Following one week of treatment, the thymii and spleens of the subject mice are harvested. The spleens are examined for splenomegaly as a sign of toxicity. Half of each thymus is frozen for RNA analysis. The other half of each thymus is processed to obtain a tissue lysate for protein analysis.

SPATIAL mRNA levels will be quantified in treated and control mice by Northern blot analysis using a radiolabeled SPATIAL cDNA as a probe. Then, the blot is stripped and re-hybridized with a cyclophilin cDNA probe. SPATIAL hybridization signals is normalized to the cyclophilin hybridization signal in the corresponding lane to allow direct quantification of SPATIAL expression across subject mice. Inhibition of SPATIAL expression by anti-sense PSC-oligos is demonstrated by a dose-dependant decrease in the normalized SPATIAL hybridization signal in treated versus control mice.

Western blot analysis is used to quantify SPATIAL protein expression in treated and control mice. Equal amounts of protein from each mouse is separated on a denaturing polyacrylamide gel, transferred to a suitable membrane, and probed with an anti-SPATIAL antibody (such as the polyclonal anti-SPATIAL antibody described in Flomerfelt et al., Genes Immun., 1:391-401, 2000). The Western blot is also stripped and re-probed with an anti-tubulin antibody. SPATIAL protein levels are normalized to tubulin protein levels to quantify SPATIAL protein levels. Inhibition of SPATIAL protein expression by anti-sense PSC-oligos is demonstrated by a dose-dependant decrease in the normalized SPATIAL protein expression in treated versus control mice.

The phenotype of the SPATIAL heterozygote mouse, as described in Example 4 and shown in FIG. 2, indicates that less-than-complete inhibition of SPATIAL expression is sufficient to increase thymocyte number in vivo. Thus, a useful dosage of anti-sense oligonucleotide for inhibiting SPATIAL expression and increasing thymocyte number is considered to be the dose of anti-sense oligonucleotide sufficient to decrease SPATIAL gene expression by at least 50%.

### Example 16

### Treatment of Bone Marrow Transplant Recipient Mice with Anti-Sense SPATIAL Oligonucleotides to Increase Thymocyte Number

This example describes representative methods of increasing thymocyte number in vivo following bone marrow transplantation using anti-sense treatment that transiently reduces SPATIAL expression in the intact thymus.

Rag2 null mice are pre-treated for different time periods, for example, on month, three weeks, two weeks, or one week with the anti-sense or control PSC-oligos given in osmotic pumps at a dosage found to be effective in Example 15. The pump is removed and one day later the treated Rag2 null mice are given bone marrow cells from a congenic (Ly5.1) wild type mouse. At 3, 4 and 5 weeks post-bone marrow transplant, the mice are sacrificed and the thymus and spleen are harvested.

Splenic B cell number is used to monitor the success of the BMT, and to assure that control and treated mice receive comparable numbers of donor cells. Cell suspensions from thymii are stained for relevant markers and analyzed by flow cytometry. Donor thymocytes in cell suspensions are identified by the Ly5.1 antibody. The numbers of donor cells in the DN1, DN2, DN3, DN4, DP and SP thymocyte subsets are calculated using antibody staining procedures described previously. The number of T cells of donor origin in the spleen and lymph nodes are quantified as a measure of thymic output.

Mice that receive pretreatment with SPATIAL anti-sense oligonucleotide are believed to have increased numbers of thymocytes as compared to control mice.

### Example 17

### Inhibition of SPATIAL Expression In Vivo by RNA Interference

RNA interference can be mediated by small temporal RNAs (stRNAs) that are transcribed as short hairpin precursors of approximately 70 nucleotides (Paddison et al., Genes Dev., 16(8):948-958, 2002). Such structures have been shown in mammals to mediate repression of endogenous mRNAs that are complementary to the sequence of the stRNAs (Paddison et al., Genes Dev., 16(8):948-958, 2002). Therefore, SPATIAL expression in vivo may be inhibited by expressing inverted repeats of different portions of the SPATIAL cDNA to form stable hairpin structures.

As described previously (see, e.g., Example 14), a cell culture system using SPATIAL-EGFP can be used to test the efficacy of candidate stRNAs in vitro. An adenovirus vector (which infects non-dividing cells) expressing different inverted repeats of the SPATIAL cDNA to form stable hairpin structures is constructed. For example, 140-base oligonucleotides containing 70-base inverted repeats of the SPATIAL cDNA are produced. Representative sequences of the SPATIAL include at least any contiguous 70 nucleic acid residues of either SPATIAL(L) or (S) (SEQ ID NOs: 1 or 3); for example, nucleotides 84-154, 154-224, 224-294, 294-364, 364-424, 424-494, 494-564, 564-634, 634-704, 704-774, 774-844, 844-914, 914-984, 960-1030 of SEQ ID NO: 3, or overlapping sequences of about 70 bases to design an inverted repeat for expression. For example, a synthetic mini-gene containing SPATIAL(L) (SEQ ID NO: 3) residues 494-564 followed by nucleic acid residues 564-494 would be expected to form a stable hairpin structure when synthesized. Synthetic mini-genes are cloned into adenoviral vectors using standard molecular biological techniques.

Adenoviral vectors are advantageous because they infect a broad array of tissue types, they can be used in mice, rats, primates, and humans, they do not result in a permanent infection as they cannot replicate, and they have been approved for use in humans as gene therapy vectors. Adenoviral vectors are commercially available (for example, from Invitrogen, Clontech, Stratagene, or Q-Biogene), and production of recombinant adenovirus is routine (see, for example, Current Protocols in Human Genetics, ed. by Dracopoli et al., New York: John Wiley & Sons, 2003, Chapter 12, Vectors for Gene Therapy, Unit 12.4, Adenoviral Vectors).

Control adenoviral vectors expressing a reporter gene, such as EGFP or LacZ (for example, pShuttle-lacZ; Clontech), are readily available for use in optimizing infection procedures. The control viruses would be genetically engineered and purified using routine methods.

To assay infectivity in vitro, 3×10⁵ target cells (such as 293T) per well of a 6-well plate are plated and increasing amounts of virus capable of expressing a control reporter gene, such as EGFP, (MOI of 0-1000 using 5-fold dilutions of virus) are added. Infection is allowed to proceed for 1 hour with rocking, then the media is aspirated and the cells are washed. Twenty four (24) to 48 hours later the transfected cells are fixed and analyzed for expression of the reporter, such as EGFP, using appropriate techniques, such as FACS analysis for EGFP expression.

Adenovirus can also be used for in vivo infection (see, for example, Current Protocols in Human Genetics, ed. by Dracopoli et al., New York: John Wiley & Sons, 2003, Chapter 12, Vectors for Gene Therapy, Unit 12.4, Adenoviral Vectors). To assay infectivity in vivo, increasing amounts of virus capable of expressing a control reporter gene (0-1×10⁷ plaque forming units (PFUs)) are injected into the mouse. Injections sites can vary but for this example a direct injection of different amounts of control virus into the thymus of anesthetized mice is preferable. Twelve (12) to 15 days later, the infected mice are euthanized and thymic expression of the reporter, such as EGFP, is analyzed.

Having established appropriate conditions for infection as described above, recombinant adenovirus carrying different SPATIAL hairpin constructs are injected in vivo and SPATIAL expression is analyzed as previously described. One measure of inhibition of SPATIAL expression in vivo is an increase in the number of thymocytes and/or peripheral T cells within about 3 weeks of treatment.

### Example 18

### Screening for Inhibitors of SPATIAL Activity Using Fetal Thymic Organ Culture

This example describes the use of fetal thymic organ culture (FTOC) to screen for agents that inhibit SPATIAL activity. In this system, the three-dimensional cellular architecture of the thymus is maintained while still allowing for direct administration of agents to be screened and easy access to thymic cells for analysis of SPATIAL expression.

Thymic lobes are excised from mouse fetuses at gestational day 16 (the day of the vaginal plug is considered as day 1). The lobes are placed on a suitable tissue culture support, such as polycarbonate membranes (Costar), in DME supplemented with 10% FCS, and, as needed, penicillin, streptomycin, 2 mM L-glutamine, and/or 50 μM 2-mercaptoethanol. Multiple FTOC can be set up in parallel in multi-well tissue culture plates, such as in 6-well culture plates.

Each agent to be screened for SPATIAL inhibitory activity, including for example, anti-sense oligonucleotides, apatamers, mirror-image aptamers, inhibitory antibodies, is added to the tissue culture medium bathing a cultured thymus. Different agents can be screened or various concentrations of one or more agents can be tested in this manner. Fetal thymii are cultured in the presence of the putative inhibitory agent(s) for sufficient time to permit the agent to inhibit SPATIAL expression; for example, useful culture times are 24, 48 and 72 hours. At the desired time points, the treated thymic lobes are harvested and prepared for analysis of SPATIAL expression.

Inhibitory agents are identified as those that decrease the expression of a SPATIAL protein or mRNA as compared to untreated cultured fetal thymii. Levels of SPATIAL protein or mRNA are measured using techniques that are well known in the art. For example, a thymic protein preparation is prepared by grinding cultured thymii in an appropriate buffer, such as phosphate-buffered saline. The soluble fraction is collected and the expression of a SPATIAL protein analyzed by Western blot. In another example, total RNA is prepared using methods known in the art and the expression of a SPATIAL RNA analyzed by Northern blot.

The effect of a SPATIAL inhibitor identified by in vitro FTOC may, but need not, be further screened by transplanting a fetal thymus treated in FTOC with one or more SPATIAL inhibitory agents under the kidney capsule of a congenic, immune-competent host mouse. Stem cells from the host enter the thymus graft and are allowed to undergo thymopoiesis for a selected period of time, for example, 7 days, 10 days, 14 days or 21 days. Then, the thymus graft is removed and the number of thymocytes counted. Thymocyte numbers are increased in transplanted thymii treated with a SPATIAL inhibitor as compared to the number of thymocytes in a co-transplanted, untreated thymus, which is placed in a separate location under the same kidney capsule.

### Example 19

### Screening Methods for Identifying Agents Useful for Improving Immune Function

This example describes representative methods of identifying agents useful for improving immune function by inhibiting a SPATIAL activity and/or interfering with an interaction between SPATIAL isoforms and its binding partners, such as Uba3. Such agents may be used as therapeutics for affecting thymic function, for example to increase the number of thymocytes produced in the thymus.

Agents that can be identified by this method include, without limitation, small molecules, polypeptides (including, for example, antibodies and proteins), peptides, nucleic acids (including, for example, nucleotides and oligonucleotides), drugs, chemicals or other compounds. Preferably, this method permits high-throughput screening of large numbers of candidate agents in order to identify those agents that specifically inhibit a SPATIAL function. Agents that specifically inhibit SPATIAL function may include; for example, agents that directly binding to SPATIAL, or prevent SPATIAL from interacting with one or more its binding partners (such as Uba3), or modify interactions that SPATIAL has with one or more of its binding partners (such as Uba3).

Screening methods may include, but are not limited to, methods employing solid phase, liquid phase, cellular, protein, peptide, virtual (in silico) and combinatorial chemistry screening techniques. Libraries useful for such screening methods include, but are not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), split and mix solid phase synthesis on beads (Furka et al., Int. J. Pept. Protein Res., 37(6):487-493, 1991; Lam et al., Chem. Rev., 97(2):411-448, 1997), and naturally occurring compounds.

In specific methods, binding assays are used to identify agents that bind to a target molecule (such as a SPATIAL isoform or fragments thereof, or Uba3 or fragments thereof) and affect the activity of the target molecule. In some instances, the target molecule is one or more functional regions of a larger molecule. For example, certain regions of the SPATIAL isoforms, which are involved in SPATIAL growth suppression activity, have been identified (see Example 8). Similarly, regions of Uba3 that are involved in an interaction between SPATIAL and Uba3 have been identified (see Examples 9-12).

Mixtures of labeled compounds, for instance radiolabeled compounds (such as, ¹⁴C-labeled compounds) can be tested for specific binding to isolated target molecules, such as SPATIAL or Uba3 or fragments of either. Purified target molecules are adsorbed overnight onto microtiter wells that are subsequently blocked with an irrelevant protein, such as casein. Labeled (for example, radiolabeled) compounds, such as compounds in one or more of the above-described libraries, are separately added to individual wells containing the target molecule. Combinations of labeled compounds can be evaluated in an initial screen to identify pools of candidate agents to be tested individually. This process is easily automated with currently available technology. The reactions are incubated for a time sufficient to permit interaction between the target molecule and the labeled compounds. The microtiter wells are extensively washed and the amount of label (such as, radioactivity) measured in the washed wells. Agents that bind the target molecule (such as SPATIAL or Uba3 and/or fragments of either) are identified by the presence of the greater-than-control levels of label (for instance, radioactivity) present in a microtiter well. Agents that bind target molecule are isolated and tested in functional assays described below. Other approaches using beads as a solid support or solution-phase screening (e.g., Boger et al., Angew. Chem. Int. Ed. Engl., 42:4138-4176, 1998; Cheng et al., Bioorg. Med. Chem., 4(5):727-737, 1996) can also be used in this approach.

In other screening methods, agents that disrupt an interaction between SPATIAL and Uba3 are identified. These assays may be performed using either solid-phase or solution-based assays. In a solid-phase assay, two components (such as SPATIAL or fragments thereof and Uba3 or fragments thereof) are mixed under conditions in which the two components normally interact. One of the components (for example, either SPATIAL or Uba3 or their respective fragment(s)) is labeled with a marker such as biotin, fluoroscein, EGFP, or enzymes to allow easy detection of the labeled component. The unlabeled component is adsorbed to a support, such as a microtiter well or beads. Then, the labeled component is added to the environment where the unlabeled component is immobilized under conditions suitable for interaction between the two components. One or more test compounds, such as compounds in one or more of the above-described libraries, are separately added to individual microenvironments containing the interacting components. Agents capable of interfering with the interaction between the components are identified as those that reduce retention of the signal (i.e., labeled component) in the reaction microenvironment, for example, in a microtiter well or on a bead for example. As discussed previously, combinations of agents can be evaluated in an initial screen to identify pools of agents to be tested individually, and this process is easily automated with currently available technology.

In still other methods, solution phase selection can be used to screen large complex libraries for agents that specifically disrupt protein-protein interactions as has been described by Boger et al. (Bioorg. Med. Chem. Lett., 8(17):2339-2344, 1998) and Berg et al. (Proc. Natl. Acad. Sci., 99(6):3830-3835, 2002). In this example, each of two proteins that are capable of physical interaction (for example, SPATIAL and Uba3 or their respective functional fragments) are labeled with fluorescent dye molecule tags with different emission spectra and overlapping adsorption spectra. When these protein components are separate, the emission spectrum for each component is distinct and can be measured. When the protein components interact, fluorescence resonance energy transfer (FRET) occurs resulting in the transfer of energy from a donor dye molecule to an acceptor dye molecule without emission of a photon. The acceptor dye molecule alone emits photons (light) of a characteristic wavelength. Therefore, FRET allows one to determine whether two molecules are interacting or not based on the emission spectra of the sample. Using this system, two labeled protein components are added under conditions where their interaction resulting in FRET emission spectra. Then, one or more test compounds, such as compounds in one or more of the above-described libraries, are added to the environment of the two labeled protein component mixture and emission spectra are measured. A decrease the FRET emission, with a concurrent increase in the emission spectra of the separated components indicates that an agent (or pool of candidate agents) has interfered with the interaction between the protein components.

Screening for agents that inhibit a SPATIAL activity can also be performed using a cellular system as described by Boger et al., (Angew. Chem. Int. Ed. Engl., 42(35):4138-4176, 2003). One advantage in this approach is that the screen is not limited to a single defined property measures a biological response. In representative assays, cells would be transiently transfected with SPATIAL-EGFP(L) or (S), or would have a stably integrated copy of SPATIAL-EGFP(L) or (S) under the control of an inducible expression systems (such as, TET-OFF, Cre-lox, etc.) such that the cells were capable of expressing a SPATIAL isoform. Because one biological activity of SPATIAL isoforms is suppression of cell growth, the cells are treated with test compound, and the cell growth characteristics of the treated cells are measured. Agents that inhibit SPATIAL activity are identified by growth of the cells treated with such agent(s). Cell growth can be measured by many methods known in the art, such as expansion of cell number, incorporation of radiolabeled molecules, such as tritiated thymidine, increases in mitochondrial activity, or increases in EGFP fluorescence. In addition, this system can also be used to confirm biological activity for candidate agents identified by other in vitro screening procedures.

### Example 20

### T Cell Response to Antigen is Normal in SPATIAL Null Mice

This example demonstrates that T cells that develop in the SPATIAL null mice do not have a gross deficiency in response to a defined antigen.

SPATIAL null and wild type littermates were immunized in the footpad with DNP-Ova in complete freunds adjuvent (Sigma). Five days later, the draining lymph nodes were harvested and CD4 T cells were purified using magnetic-bead depletion columns (e.g., Beaulieu, et al., J. Immunol. Meth., 180(2):225-236, 1995) T cell-depleted spleen cells from non-immunized mice were pulsed with DNP-Ova. Cultures were set up with 4×10⁵ CD4 T cells and 1×10⁵ APC and were allowed to grow for 72 hours. Tritiated thymidine was added during the last 18 hours of the culture to assess proliferation.

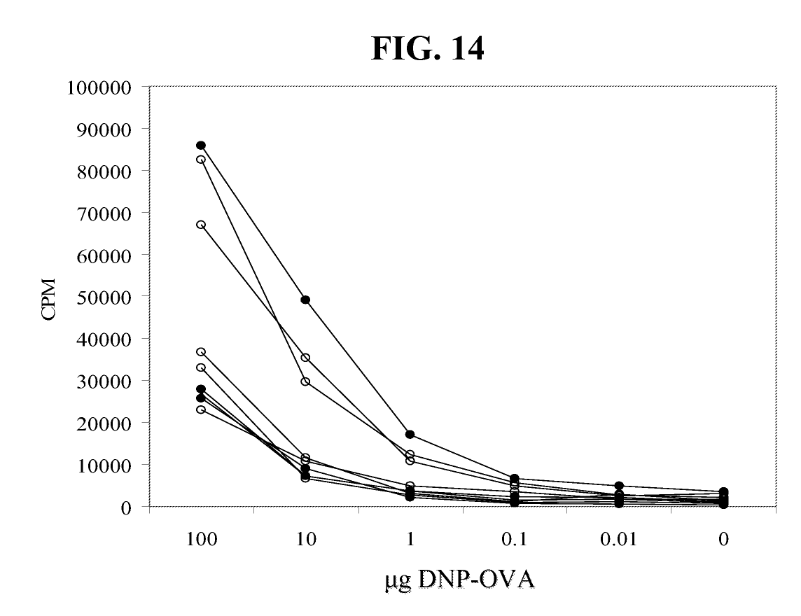

As shown in FIG. 14, there was no qualitative difference in the T cell response between SPATIAL null and wild type mice. Therefore, T cells that develop in the SPATIAL null mice do not have a gross deficiency in response to DNP-Ova.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

We claim:

1. A method of improving immune function in a subject, comprising inhibiting a Stromal Protein Associated with Thymii And Lymph node (SPATIAL) activity in a subject and thereby improving immune function in the subject, wherein immune function in the subject has been compromised by other than age-related immunodeficiency.

2. The method of claim 1, wherein the compromise of the immune system results from administration of a toxin to the subject, infection of the subject with an infectious agent, or treatment of the subject with radiation therapy, or as a result of a disease.

3. The method of claim 1, wherein inhibiting a SPATIAL activity results in increasing thymocyte number in the subject.

4. The method of claim 1, wherein inhibiting a SPATIAL activity comprises inhibiting SPATIAL gene expression or SPATIAL polypeptide activity.

5. The method of claim 1 further comprising administering to the subject a therapeutically effective amount of an agent that inhibits a SPATIAL activity.

6. The method of claim 5, wherein the agent comprises a small inhibitory RNA, an anti-sense nucleic acid, a ribozyme, an aptamer, a mirror-image aptamer, an Uba3 peptide, a SPATIAL peptide, an Uba3-specific antibody, or a SPATIAL-specific antibody.

7. The method of claim 5, wherein the agent inhibits an interaction between SPATIAL and Uba3.

8. The method of claim 7, wherein the agent comprises an Uba3 peptide, a SPATIAL peptide, an Uba3-specific antibody, a SPATIAL-specific antibody, an aptamer or a mirror-image aptamer.

9. The method of claim 7, wherein the agent comprises at least 15 consecutive amino acids of SEQ ID NO: 6.

10. A method of increasing thymocyte number in a subject having disease-associated T cell deficiency, comprising administering to the subject a therapeutically effective amount of an agent that inhibits a SPATIAL activity, thereby increasing thymocyte number in the subject.

11. The method of claim 10, wherein inhibiting a SPATIAL activity comprises inhibiting SPATIAL gene expression.

12. The method of claim 10, wherein the agent comprises a small inhibitory RNA, an anti-sense nucleic acid, a ribozyme, an aptamer, a mirror-image aptamer, an Uba3 peptide, a SPATIAL peptide, an Uba3-specific antibody, or a SPATIAL-specific antibody.

13. The method of claim 10 wherein inhibiting SPATIAL activity comprises inhibiting a SPATIAL polypeptide activity.

14. The method of claim 13, wherein the agent inhibits an interaction between SPATIAL and Uba3.

15. A method of increasing thymocyte number in a subject having a thymus, comprising administering to the subject a therapeutically effective amount of an agent that interferes with an interaction between SPATIAL and Uba3, thereby increasing thymocyte number in the subject.

16. The method of claim 15, wherein the agent inhibits an interaction between SPATIAL and Uba3.

17. The method of claim 15, wherein the agent comprises an Uba3 peptide, a SPATIAL peptide, an Uba3-specific antibody, a SPATIAL-specific antibody, an aptamer or a mirror-image aptamer.

18. The method of claim 15, wherein the agent comprises at least 15 consecutive amino acids of SEQ ID NO: 6.

19. The method of claim 15, wherein the subject is immunodeficient.

20. The method of claim 19, wherein the immunodeficiency is cellular immunodeficiency or combined immunodeficiency.

21. The method of claim 19, wherein the subject has received a bone marrow transplant, chemotherapy or radiation therapy.


SEQUENCE LISTING (7 sequences)

SEQ ID NO: 1 - DNA, 933 bp, Mus musculus
  CDS (84)..(677): Coding sequence
tcttgaggttgccaatttttttttttttttttttttttttttttttttttggtttggggagaaacttgtgttggaaccagcccctgtttctggggaatgtatataaggggagtttagcacctcgtagggatgaggtgactagtccaaaggcagagccccagccagagacgaagccggagaaccttccaaggagccacggggatgttgggctccagaaagagactgtggtcccaggcattgtggatttcgagctgatccatgaggagctgaagaccacaaagccccaaacatcacaaccaacacccagtgcctaccgctttggacgcctaagccaccattccttcttctcgaggcaccacccccaaccacagcgagtgactcatatccaagatatcgctgggaagcctgtctgcgtggtcagggacgagttctctctgtcggccttgactcagcccacattcttatcccgctgtctgatggggatgcccaccatctctgtccccattggggatccacagtccaatcggaacccccagctttctacttctgacacctggaggaagaaactgaaggacctggcttcccgagtgactgtcttcactaaggaaatccagccaaagcccgatgaggttggtgttgcacaaagaatggagcctagaaaaaaaaggccttcttaagtctccccaatgctcagctgctggcacgggaggggaaggaccctcataacctcgaaggtgacagcgaaaatcaaagaaacacaaaatcacacctagcagagaaatccaagaagggttcccagaaacaccctctaaagcaactgttcccaacctgtctaatgccttgacccttgaatacagtttctcacactgcagtaacccctgcccccgaaataaaattattttcattactacttcaaaaaaaaaaaaaaaaaaa

SEQ ID NO: 2 - protein, 197 aa, Mus musculus
LFLGNVYKGSLAPRRDEVTSPKAEPQPETKPENLPRSHGDVGLQKETVVPGIVDFELIHEELKTTKPQTSQPTPSAYRFGRLSHHSFFSRHHPQPQRVTHIQDIAGKPVCVVRDEFSLSALTQPTFLSRCLMGMPTISVPIGDPQSNRNPQLSTSDTWRKKLKDLASRVTVFTKEIQPKPDEVGVAQRMEPRKKRPS

SEQ ID NO: 3 - DNA, 1035 bp, Mus musculus
  CDS (84)..(779): Coding sequence
tcttgaggttgccaatttttttttttttttttttttttttttttttttttggtttggggagaaacttgtgttggaaccagcccctgtttctggggaatgtatataaggggagtttagcacctcgtagggatgaggtgactagtccaaaggcagagccccagccagagacgaagccggagaaccttccaaggagccacggggatgttgggctccagaaagagactgtggtcccaggcattgtggatttcgagctgatccatgaggagctgaagaccacaaagccccaaacatcacaaccaacacccagtgcctaccgctttggacgcctaagccaccattccttcttctcgaggcaccacccccaaccacagcgagtgactcatatccaagttacaggaagagaggacctggagcactccctgcccctcaccacctctttccagctccttcaagctcctggggtccagcccatggatctcactccctctgcagatatcgctgggaagcctgtctgcgtggtcagggacgagttctctctgtcggccttgactcagcccacattcttatcccgctgtctgatggggatgcccaccatctctgtccccattggggatccacagtccaatcggaacccccagctttctacttctgacacctggaggaagaaactgaaggacctggcttcccgagtgactgtcttcactaaggaaatccagccaaagcccgatgaggttggtgttgcacaaagaatggagcctagaaaaaaaaggccttcttaagtctccccaatgctcagctgctggcacgggaggggaaggaccctcataacctcgaaggtgacagcgaaaatcaaagaaacacaaaatcacacctagcagagaaatccaagaagggttcccagaaacaccctctaaagcaactgttcccaacctgtctaatgccttgacccttgaatacagtttctcacactgcagtaacccctgcccccgaaataaaattattttcattactacttcaaaaaaaaaaaaaaaaaaa

SEQ ID NO: 4 - protein, 231 aa, Mus musculus
LFLGNVYKGSLAPRRDEVTSPKAEPQPETKPENLPRSHGDVGLQKETVVPGIVDFELIHEELKTTKPQTSQPTPSAYRFGRLSHHSFFSRHHPQPQRVTHIQVTGREDLEHSLPLTTSFQLLQAPGVQPMDLTPSADIAGKPVCVVRDEFSLSALTQPTFLSRCLMGMPTISVPIGDPQSNRNPQLSTSDTWRKKLKDLASRVTVFTKEIQPKPDEVGVAQRMEPRKKRPS

SEQ ID NO: 5 - DNA, 2117 bp, Mus musculus
  CDS (40)..(1365)
  variation (336)..(336): results in encoded ′Xaa′ at location 99 stands for Val.
aagaaaagaaggagaatagaggagctgctggctgagaaaatggctgttgatggtgggtgtggggacactggagactgggaaggtcgctggaaccatgtaaagaagttcctcgagcggtctggacccttcacacaccccgatttcgaaccaagcactgaatcactccagttcttgttagatacatgtaaagttctagtcattggagctggtggcttaggatgtgagcttctgaaaaatctggcattatctggttttagacagattcatgttatagacatggacactatagatgtttccaatttaaatagacagtttttatttaggcctaaagatgtyggaagacccaaggctgaagttgctgcagaattcctaaatgacagagttcctaactgcaacgtggtmccacatttcaacaagatwcaagattttaacgacactttctaccgacaatttcatatcattgtatgtggcctggactctatcatagcgagaagatggatcaatggaatgctgatatctcttctaaattatgaagatggtgtgttggatccaagctccattgtacctttgatagatggggggacagaaggctttaaagggaatgcccgagtgattttgcctggaatgaccgcttgtattgagtgcactctggaactttacccaccacaggtcaatttccccatgtgtaccattgcatctatgccyaggctcccagaacactgtatcgagtatgtgaggatgttgcaatggcctaaagagcagccttttggagatggggttccattagatggagatgaccctgaacatattcagtggattttccaaaagtccatagagagagcatcacaatataatattagaggcgttacctacagactcactcaaggggtggtaaaacgaatcattcctgcagtagcttctacaaatgcagtcattgcagctgtgtgtgccactgaggttttcaagatagctacaagtgcgtacattccccttaataactacctggtattcaatgatgtagatgggctgtacacttacacgtttgaagcagagagaaaggaaaactgtccagcatgtagccaacttcctcaaaacattcagttttccccatcagctaaactacaggaggtcttagactacctaaccaacagtgcttctctgcaaatgaagtctccggctatcacagccacattagaggggaagaacaggacactttacttacagtcagtaacgtctattgaagaacgaaccaggcccaatctttccaaaacattaaaagaactgggactagttgatggacaagaactggctgttgctgatgtcactacaccacagactgtactattcaaacttcattttacttaaggaaaataaatctgcacataatagaaaattcatagaaataatatactttataaatgatatgaaattgaagagcctggaagatgaggcagaggggaacatccaagaaaggaaatttaattggtgtcatttttagcattagtgtggctagaatttgacttttatatatatacatatatataaaaaaggactgactcttttttaactttataagtttctcttgaagactgaactttggggttgggctagcaagcattttcattttattactatggaaagctatgccttcaggagagattatgaacaagtgtgttgcttctttaaagcaggacaaacactgtcttgtgtgtgagtttgttgtggtcaaagagcatattcctcagcgtgtatctgaaatccacatgtgtagaaatgtctcctgggatggaaatgaggagctatgtctgaagaatagtaaatattcacagcctgacatctagagtatatcaaacataggcagtgtcttcattgctactcatataattgtgactatccatgtgtgtattaattattgcagaatttaacttgtccatgataatttgtaaacagtattatagattcatacctgtgcatgaaaatacaaaatattttcatgtatttgtttgcaatgccacagagaccagtatgcacaaatttaaaccaagacatggctgttcaaagaaaattaatgtttaaacagttatcattgatgcttttgcactatttattaataaaat

SEQ ID NO: 6 - protein, 441 aa, Mus musculus
  misc_feature (99)..(99): The ′Xaa′ at location 99 stands for Val.
MAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDXGRPKAEVAAEFLNDRVPNCNVXPHFNKXQDFNDTFYRQFHIIVCGLDSIIARRWINGMLISLLNYEDGVLDPSSIVPLIDGGTEGFKGNARVILPGMTACIECTLELYPPQVNFPMCTIASMXRLPEHCIEYVRMLQWPKEQPFGDGVPLDGDDPEHIQWIFQKSIERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFT

SEQ ID NO: 7 - DNA, 5807 bp, Mus musculus
ttaggtgtcctagttagggttactattgacatgatgaaagaccgtgaccaaagcaacttaaggaggaaaggctttatttggcttacacttccataccacagctcaccatcaaaggaatcaaagggagtcagggcaggaacctggaggcaagagctgatgcagaagccatggaaagatgctgcttcctagcttgctccccctggcttgctcagctggctttcttgctcagctccgaggtgactccacccttcctctaccaatcattaattaggaaaatgcctaacaggcttctctacagccccatcttaaggaagccttttctcaagtgaggctccctcctctccaatgactttagctggcattaaattgacataaaactagccagcacagggctgaccagctcagctgccacccaggcccgggactcaaggctttgagttggcccacccccaaatctacatcatctgtgaactgttggggcatgtgaaaatgctgtgcctgttgattcaaagctacatggtctccatgacacagggtgacaacgagatatctgagaggtgtcccaatgaggatctaatattgatggagtcacagaagccagacaccttgaaccaaactaatgactcattgcaatgaatatttgcaagtgaagatgtgtggacagagggttatactgtgggatatactgtaacacattacatcttccacgatgttttcttttctctctttctctctctctctctctctctctctctctctctctctctctctctctctctctgtgtgtgtgtgtgtgtgtgtgtgtgtgtgtgtgtgtgtgtgtgttatttgaggaggggttgcaggggcagagggcagatatgaggggatgagctggactggggtgcctgatgtgaaacaaagaatcaataaaatgttaatacaaaggcccggagagatggcttagcagttaagagcaccaactgctcttccaaaggtcctgagttcaaatcccagcaaccacatggtggctcacaaccatccataatgagatctgacatcctcttcttggatgtctaaagatagctacagtgtacttacatataataaataaatgaatctttcggctggagcaagcagggctggagagagaagaggtggtgtctgaagacagctatagtgtacttacatacaataaacaatcttttaaaaataatgataaaacaaatgaggatctacccagcacactgagctaatgacccaagagtgtcagggagccagctaaatctgtccagtgctgatactgtaggtgtgacaacactgtgacccagcttttctgtttgttttgtttttttgacacgagggttctggaggttgaactcatgccttcacgcttgcagggcaaacactttgcagacgaagccatctccccagcccccctcttagagtatctccagcatgcacatcattaggcactctactcacaacaatccatttattcttcagagcaagcccggagtaggcgacagcactcgttccccagccccagcctacacagtaatcggtaagagatcagatttgaagccacatagtgttgtaagcactttcatgcgtgttaagtcgatttccaagggacaataagtggggaccccatttcagccctaccagacctgagcccctaagagtaattaataccctaccccgagcctgacctcttgagcaaaattttggaaaaaaaaatgcagatatgctagaaaactgtatataatgtgcctaactggttagatttagatgaaacttggggagattgtagaaaataagcatccactattcctatgagatcaggaacttggttttataaacatttatgtcattcctgtgttagcaggaactctgataggactagaggtatgaagttcagagcctttgggtagactctctgaaatcacaacaggataaatggcagaatgggattttaacaaggtgctttgcgctccatacccctggcaacacaggcattttagctaactgaagaccttaaccttccctccctgccagcccttcaagtgaagccacagggacccaccagatcctatgattgggttcctcttctctacctaagaaataaatagctccctcaggactgatcccaagaatcaaagctcccaaaggctcaactatgcacagcagcatcgacctctggcaagctggcaagaccttggacagattttcaccttctgactggttcgactgggtagttaacctacttgtgtgggtccgtactggaagtgcttggataagaagcagaggaggaatttcaagggataagaaagagtcctctgtgagctgtctgcctgccctacccccaactccccaggaaggggaaagatgaactccacagtggtgggggagaaaggtgttgggctatgttctggagttgtggggaaggggtaacctagcaaccactgggctcagcatagtcacaaagcaacaggtttgcccacaattccgacaagccttacccttgattagtcttcttctccatcttggtcaggcaccaggggcttcttgctcatatgctatagtggacatcttcctggtgagttcagggccctctcaggagggtagggaatgaaatgggatctggctccaggtgacaacaaaggaattgtggggggataagagggttcctgtggttacattggtggctcccttcagatctcttgtaactgactatcaacgagcccaggatcagaggcaggggagggcagggatgggctgcagtgacctcatctgtctctcttcctaggtcctactgacccttgtaggtgggtgttgattcttaatcttattgaagaatttaatggctcatgtctaacattcatgaagaaaaaaaatcaagatgcaagtatactcgtacatcttacatgtagatactgcttcagactactcccatgaccactgcagacacatacaggcacggaggcatgcacgcatgcagacacacacacacacacacacacacacacacacacacacacgaatttagacctaaggaaaaatggatttcttgctgttgtaatggtcaagagtgtttgagagtttctattctaaagagcactttttcctgtaggcgggtggatgagggactttcaaatgccctgtcttcaagcatgaaacatccaagaaccccagcccctaccatcaatgatacatgcatggagtttcctgaagagggagccctgctcactctttttagacaaccgatcagcagcctgtgagccagatgactatgctggcccttgactgtgcagcctcaaatgaagccaacccatcacctactactaacctgatggtggtggcaccgaggcatctcagagagccttgcatatctgtggttccagcacctttctcagaactcacttgcaccaggtccccaacgcttgcatagggcctgtagatttaaccataattctggctttgatatctgtcgtctttcatccatttcttttcattggtttacttacaaatgcactgagctgagtaagagccacctgctcaaggaccacagcaccaccactaactcatctcctgtgtgctctcctccttgtccctaaaatgccactagggaaatgtccttagttattttattgtttgatctcatgtttgtgataattctgtttgcctgggaatttgtgtgtgtgtttgggggtggatgtgtatgagtttaggtatatgtatgtatgggtgtgtatgagtgtgtgggtatggtgtgtggttttttgatgtgtctgtatgtgtagatgaatgtgtgagaatgagtttgtgtgtgtttgtgtatgtgtatggatgtatgtgtgtggtgtgtggtgtgtgtatgtgtggcaatatattgtgtatggatgggtgtgtgtgtatggatgtatgtgtatgggtatgtgtgtgtatgtatgtgtatgatgtgtgtatatgagtgagtgtgtgtgtgtatggtatgtgtgtttatagtgtgtggatgtgtggatgaatgtgtgtgaatgggtttgtgtgtatttgtagtgtgtgtgtgtgtgtgtgtgtgtgtgtgtgtgtgtgcgcgcgcgcgcatgtagaggtcagagaacaacttggaggagttaattttctctttacctcgtgtgggtggcaagttcctcagcctgttgagccatcttactggtcctgtgaacttttacttttaaaaagcttttcaaagctgggcatggtgggtctgcccttaacccctgactctggatcagacacaggtagatgtctgtaagttggagaccaaggttggcctggtctccatatcaagttccagggcagttagggctacatagtgagaccctatgccattgcttctcaaccttcctaatgcggtgaccctttaatacagtccctcatgttgtggtgacccccaccataaaattgtttcattgcttcttcatagctgtaattttgatactgttatgaattgtaatgtaactatctcatatttgactcctgtgagaaggtcatttgaccttccaagggggtgcagctcacaggttgagaactgytcccctgtgttgcttccaaaccatgccaagcactatgtttgcatggtttgtccatgctgtggctatcactacggttcactgcatctcattgatggtctgaacaaagccccagctttgttagacataggtgaaatcactgggttttggctgattcagaccctctgaatagtgctactctcccagacagcccatgtcacacagctgtaggagtgtctcttgtgtcagggagagaaatggctgggtgatttggtgtccaacttaaccaggcactgtccccctctcactctcagatcgcatgttccagtttatacacccgccaccaggccaccagagctgtgccatccatcaaagacacggacattccgagtctagtctttacatctcttattgcaaaccctgatatgactacagaggtgaaccagctgtctgagcatcctctagtgaggtgaggccctaaatggacacgggctggagagagcatgtaagaccaaaggggagaggaggcaggatagaagacaagaatagccttggaggaacttggaaaaaggtggttcaagttccagggaaagccatctaccaagggcctcggtagctgtgttgacacaggtctctgaaacacagaaggaggtagggatacagcagaagggctctggagtttctggagacgtcctctttatttgggagtagaggaagcctcttactggcacttcagagaggtttgctttctttctttaagacctggattgcctagagctgaaaggaaggagaatggaacaggctacacagggggctgcccagcaacatcttcccagcaatcaggggaggcacgccttccttagcatatcccctgcccacctccattccatcaggagacctcccaccagccagggccagcaagagaagaaagtgaaaaaagccaccaggcaggtggtagagtgcaggctctccttcagggacagacctcggcttccttcagggacagagactaaacatggggtggcttgctgggaacctgagacagctggtctccggtcccacccaagaagacaagagttggacgcttctcgtgtcccgtgtccgtcccccccccccagctccctcccctttggtcagcttcgcgtctcccgggaaggaccacgtgggcaaaggactcactaggaccaccctggtcttggctctagttctgacccctgggtgctgaaactaagataataaggacacagtcgggagggtaacagcctgaagctggtt